(19) United States
(12) Patent Application Publication
(10) Pub. No.: US 2022/0241333 A1
(43) Pub. Date: Aug. 4, 2022

(54) MODULATION OF T CELL CYTOTOXICITY AND RELATED THERAPY

(71) Applicants: Cancer Research Technology Limited, London (GB); University College London, London (GB)

(72) Inventors: Sergio Quezada, (US); Karl Peggs, (US); Anna Sledzinska, (US); Richard Jenner, (US); Felipe Galvez Cancino, (US); Maria Vila de Mucha, (US)

(21) Appl. No.: 17/622,190

(22) PCT Filed: Jul. 3, 2020
(86) PCT No.: PCT/EP2020/068912

§ 371 Date: Dec. 22, 2021

(30) Foreign Application Priority Data
Jul. 3, 2019 (GB) 1909573.6

(51) Int. Cl. A61K 35/17; A61P 35/00; C07K 14/725; C12N 15/10

(52) U.S. Cl. CPC A61K 35/17; C12N 15/102; C07K 14/7051; A61P 35/00

(57) ABSTRACT

The present invention provides an engineered T cell for use in a method of treatment of a proliferative disorder in a mammalian subject, wherein the T cell has been engineered (i) to overexpress BLIMP1 and/or (ii) to knock-out or decrease expression of BCL6. Further provided is a BCL6 inhibitor for use in a method of enhancing immunotherapy in a subject having a proliferative disorder. Also provided are related methods of treatment employing the engineered T cell and/or BCL6 inhibitor.

This application claims priority from GB1909573.6, filed 3 Jul. 2019, the contents and elements of which are herein incorporated by reference for all purposes.

Field of the Invention

The present invention relates to products and methods for modulating, including enhancing, T cell cytotoxicity. In particular, enhancement of CD4+ T cell cytotoxicity is disclosed for use in the treatment of proliferative disorders, such as cancer.

Background to the Invention

Shortly after the definition of the classical T helper (Th) type 1 (Th1) and type 2 (Th2) lineages (Mosmann et al., 1986), it was reported that mycobacterial antigens could induce the development of cytotoxic CD4⁺ T cells (Mustafa and Godal, 1987; Ottenhoff, 1988). Such cytotoxic CD4⁺ T cells have been found in both mice and humans in a wide range of pathological conditions including CMV, HIV, EBV, influenza and dengue virus infection (Juno et al., 2017), as well as in tumours. For example, melanoma-reactive CD4⁺ T cells acquire cytotoxic activity and mediate direct recognition and elimination of large melanoma lesions in transplantable and spontaneous mouse melanoma models (Quezada et al., 2010; Xie et al., 2010). Similarly, NY-ESO-1-specific CD4⁺ T cells isolated from stage IV melanoma patients are able to lyse melanoma cells expressing the cognate antigen in the context of MHC-II. Moreover, the number of these cells in the blood increases after treatment with Ipilimumab (anti-CTLA-4) (Kitano et al., 2013). GzmB-expressing CD4⁺ T cells are present within the CD25−CD127− compartment of blood in patients with metastatic uveal melanomas and breast cancer and expanded after chemotherapy (Péguillet et al., 2014). Cytotoxic CD4⁺ T cells are also found in the peripheral blood of patients suffering from leukaemia (Haigh et al., 2008).

Several attempts have been made to define a set of surface markers allowing cytotoxic cells to be distinguished from other Th subsets, but there is no consensus as to whether such markers exist. Indeed, it has become widely accepted that CD4⁺ T cell lineages exhibit a degree of plasticity, with cells simultaneously expressing markers of more than one Th lineage and retaining the ability to switch phenotypes during their lifespan (Dupage and Bluestone, 2016). In keeping with this, GzmB-secreting cytotoxic CD4+ T cells co-express activation markers, cytokines and transcription factors associated with different Th subsets (Takeuchi and Saito, 2017; Tian et al., 2016). Perforin-expressing human CD4⁺ T cells have been shown to co-express TNFα, IFNγ and Granzyme A (GzmA) (Appay et al., 2002). A similar Th1 cytokine profile was reported to be expressed by cytotoxic CD4⁺ T cells recognizing EBV-transformed B cells (Haigh et al., 2008).

The transcription factors required for the development of cytotoxic CD4⁺ T cells in vivo remain similarly unclear. T-bet and Eomes are potential candidates due to their well-established role in controlling Th1 responses and inducing GzmB and Perforin expression in CD8⁺ T and NK cells (Evans and Jenner, 2013; Glimcher et al., 2004; Lupar et al., 2015). T-bet also directly binds and activates GZMB, PRF1 and NKG7 in CD4⁺ T cells in vitro (Kanhere et al., 2012). Although T-bet overexpression increased the lytic potential of a CD4+T helper cell line in vitro (Eshima et al., 2018) in vivo studies suggested that Eomes has higher potential in inducing GzmB in Th cells (Qui et al., 2011). In contrast, recent studies in an adenovirus infection model showed that the cytotoxic program does not correlate with T-bet or Eomes expression and instead is in direct opposition to the Bcl6-driven follicular helper T (Tfh) cell differentiation program (Donnarumma et al., 2016). These virus-induced cytotoxic cells also exhibited higher expression of the transcriptional repressor Blimp-1, which was previously shown to downregulate Bcl6 and Tcf1 expression in CD4+ T cells (Choi et al., 2015; Fu et al., 2017; Johnston et al., 2009; Wu et al., 2015). Potentially related to this, human CMV-specific cytotoxic CD4+ T cells have been reported to express high levels of the Blimp1 homolog Hobit (Oja et al., 2017). Such a complex network of transcription factors implicates an equally long list of potential environmental factors regulating cytotoxic cell development, ranging from T cell receptor (TCR) signal strength to members of the common gamma (cγ) chain cytokine family or IFNα (Hua et al., 2013). In vitro, exogenous IL-2 is required to increase the lytic potential of CD4⁺ T cells in response to low antigen dose (Brown et al., 2009), and IL-2 was reported to be a potent inducer of Perforin and GzmB expression in CD8⁺ T cells (Janas et al., 2005). IL-2 was also shown to oppose the differentiation of Tfh cells by downregulating Bcl6 expression (Ballesteros-Tato et al., 2012), hence playing a role in controlling the Bcl6/Blimp-1/Tcf1 balance (Fu et al., 2017).

WO2018/108704 describes 6-amino-quinolinone compounds and derivatives thereof as BCL6 inhibitors for the treatment and/or prevention of oncological diseases. In particular, the compounds are proposed for use in treatment of BCL6 over-expressing diffuse large B-cell lymphoma (DLBCL).

Quezada et al., J. Exp. Med., 2010, Vol. 207, No. 3, pp. 637-650 describes tumour-reactive CD4⁺ T cells that develop cytotoxic activity and eradicate large established melanoma after transfer into lymphopenic hosts.

There remains an unmet need for therapeutic agents and methods that enhance immune-mediated cancer therapy. The present invention addresses these and other needs, and provides related advantages as described herein.

### BRIEF DESCRIPTION OF THE INVENTION

Broadly, the present invention relates to the modulation of the BLIMP-1/BCL6 axis to enhance CD4⁺ T cell cytotoxicity and thereby to enhance anti-cancer therapy. In particular, the present disclosure relates to the use of pharmacological agents to enhance an immune response against a tumour and to the use of engineered T cells (including chimeric antigen receptor T cells (CAR-T) and neoantigen reactive T cells (NAR-T)) that exhibit enhanced cytotoxic activity for the treatment of a tumour. As disclosed in detail herein, the present inventors have found that Blimp-1 is a key factor controlling he development of cytotoxic CD4⁺ T cells in vivo following anti-CTLA-4 antibody mediated Treg depletion, and that Th-ctx cells are crucial for eradicating sarcoma. Moreover, as shown in the experimental examples herein, gene knock-out of BCL6 (which has an inverse expression relationship with Blimp-1), gene editing of BCL6 using CRISPR and pharmacological inhibition of BCL6 using small molecule BCL6 degraders were all found to enhance GZMB expression in CD4⁺ T cells and CD8⁺ T cells indicating enhanced cell killing activity. It follows from the data disclosed herein that manipulation of the Blimp-1/BCL6 axis to enhance Blimp-1 or downregulate BCL6 will augment T cell-driven anti-cancer effects.

Accordingly, in a first aspect of the invention, there is provided an engineered T cell having reduced BCL6 expression and/or enhanced BLIMP-1 expression for use in a method of treatment a proliferative disorder.

In some embodiments the T cell comprises a chimeric antigen receptor T cell (CAR-T), an engineered T cell receptor (TCR) T cell or a Neoantigen-reactive T Cell (NAR-T).

In some embodiments the T cell is autologous to said subject.

In some embodiments the proliferative disorder comprises a solid tumour. In particular, the solid tumour may be a cancerous tumour including a primary tumour or a metastasised secondary tumour.

In some embodiments the solid tumour comprises a melanoma or a sarcoma.

In some embodiments the BCL6 knock-out or downregulation and/or the BLIPM-1 overexpression is engineered by CRISPR/Cas9-mediated gene editing, transcription activator-like effector nucleases (TALENs) transient downregulation using short hairpin RNA (shRNA), small interfering RNA (siRNA), microRNA (miRNA) or RNA constructs for overexpression. Editing of the BCL6 gene or PRDM1 gene (encoding BLIMP-1) and/or a regulatory element (e.g. promoter) of the same are specifically contemplated.

In some embodiments the engineered T cell is for use in a method of treatment that further comprises simultaneous, sequential or separate administration of an immune checkpoint inhibitor therapy. In some cases immune checkpoint inhibitor therapy may comprise CTLA-4 blockade, PD-1 inhibition, PD-L1 inhibition, Lag-3 (Lymphocyte activating 3; Gene ID: 3902) inhibition, Tim-3 (T cell immunoglobulin and mucin domain 3; Gene ID: 84868) inhibition, TIGIT (T cell immunoreceptor with Ig and ITIM domains; Gene ID: 201633) inhibition and/or BTLA (B and T lymphocyte associated; Gene ID: 151888) inhibition. In particular, the immune checkpoint inhibitor may comprise: ipilimumab, tremelimumab, nivolumab, pembrolizumab, atezolizumab, avelumab or durvalumab.

In some embodiments the engineered T cell may comprise a modified form of Tisagenlecleucel or Axicabtagene ciloleucel, which has been modified to overexpress BLIMP1 and/or to knock-out or decrease expression of BCL6.

In some embodiments the engineered T cell is a CD4⁺ T cell having cytotoxic activity. Cytotoxic activity may be assessed by suitable assays for the cytotoxic phenotype, such as granzyme B (GzmB) expression as described in the Examples herein. In some embodiments the engineered T cell is a CD4⁺ effector T cell.

In a second aspect the present invention provides a method of treatment of a proliferative disorder in a mammalian subject, comprising administering a therapeutically effective amount of an engineered T cell to the subject in need thereof, wherein the T cell has been engineered (i) to overexpress BLIMP1 and/or (ii) to knock-out or decrease expression of BCL6.

In some embodiments the T cell comprises a chimeric antigen receptor T cell (CAR-T), an engineered T cell receptor (TCR) T cell or a Neoantigen-reactive T Cell (NAR-T).

In some embodiments the T cell is autologous to said subject.

In some embodiments the proliferative disorder comprises a solid tumour. In particular, the solid tumour may comprise a melanoma or a sarcoma.

In some embodiments the T cell is engineered to knock-out or downregulate expression of BCL6 prior to being administered to the subject.

In some embodiments the T cell is engineered to overexpress BLIMP-1 prior to being administered to the subject.

In autologous T cell therapy the T cells removed from the subject are typically engineered ex vivo, e.g. to target the T cells to an antigen expressed on the tumour (for example to insert a gene encoding a chimeric antigen receptor). Advantageously in accordance with the present invention the T cells may be additionally engineered during or as part of this ex vivo stage to downregulate BCL6 expression and/or upregulate BLIMP-1 expression before the T cells are then returned to the subject.

In some embodiments the BCL6 knock-out or downregulation and/or the BLIPM-1 overexpression is engineered by CRISPR/Cas9-mediated gene editing, transcription activator-like effector nucleases (TALENs) transient downregulation using short hairpin RNA (shRNA), small interfering RNA (siRNA), microRNA (miRNA) or RNA constructs for overexpression.

In some embodiments the method further comprises simultaneous, sequential or separate administration of an immune checkpoint inhibitor therapy to the subject. Such combination therapy may give rise to synergistic enhancement of anti-tumour effects. In particular, the immune checkpoint inhibitor therapy may comprise CTLA-4 blockade, PD-1 inhibition, Lag-3 (Lymphocyte activating 3; Gene ID: 3902) inhibition, Tim-3 (T cell immunoglobulin and mucin domain 3; Gene ID: 84868) inhibition, TIGIT (T cell immunoreceptor with Ig and ITIM domains; Gene ID: 201633) inhibition, BTLA (B and T lymphocyte associated; Gene ID: 151888) inhibition and/or PD-L1 inhibition. For example, the immune checkpoint inhibitor may be selected from: ipilimumab, tremelimumab, nivolumab, pembrolizumab, atezolizumab, avelumab and durvalumab.

In some embodiments the engineered T cell is a CD4⁺ T cell having cytotoxic activity.

In a third aspect the present invention provides a BCL6 inhibitor for use in a method of enhancing immunotherapy in a subject having a proliferative disorder. In particular, the BCL6 inhibitor may be for use in combination with T cell therapy, such as the engineered T cell of the first aspect of the invention. In certain cases, anti-cancer therapy may involve simultaneous, separate or sequential administration of a BCL6 inhibitor and an engineered T cell of the first aspect of the invention to a subject having or suspected of having a proliferative disorder. In this way combination therapy may provide superior therapeutic outcome compared with treatment with a BCL6 inhibitor alone or T cell therapy alone. Without wishing to be bound by any particular theory, the present inventors believe that pharmacological inhibition of BCL6 in vivo may augment the anti-cancer effects of endogenous and administered T cells.

In some embodiments the immunotherapy comprises immune checkpoint inhibition, an anti-tumour vaccine or an autologous T cell therapy.

In some embodiments the proliferative disorder comprises a solid tumour. In particular, the solid tumour may comprise a melanoma or a sarcoma.

In some embodiments the amount or dose of BCL6 inhibitor administered to the subject is sufficient to enhance cytotoxic activity of CD4⁺ and/or CD8⁺ T cells in the subject.

Without wishing to be bound by any particular theory, the present inventors consider that the use of a BCL6 inhibitor to enhance immunotherapy offers distinct advantages relative to the use of BCL6 inhibitors for direct anti-cancer targeting, such as described in WO 2018/108704. In particular, use of anti-cancer agents for direct cell killing typically requires dosing at or close to the maximum tolerated dose. Indirect anti-cancer effects via augmentation of immune cell-based tumour killing, on the other hand, are considered to be achievable at lower doses. Moreover, the targeting of BCL6 on T cells to enhance tumour killing does not require the cancer cells themselves to express or over-express BCL6. Thus, the BCL6 inhibitors for use in accordance with the present invention and the methods of treatment comprising administering BCL6 inhibitors of the present invention may, in some embodiments, be agnostic to the presence of or degree of expression of BCL6 on or by the cancer cells. In particular, in some embodiments the BCL6 inhibitor may be for use in a method of enhancing immunotherapy in a subject having a proliferative disorder that comprises a BCL6 negative tumour or a tumour that does not over-express BCL6.

In a fourth aspect the present invention provides a method of treatment of a BCL6-negative tumour in a mammalian subject, comprising administering a therapeutically effective amount of a BCL6 inhibitor to the subject, wherein the BCL6 inhibitor enhances cytotoxic activity of one or more T cells in the subject and thereby treats the BCL6-negative tumour. In some embodiments the BCL6-negative tumour comprises cancer cells that do not over-express BCL6 and/or that do not carry mutations in the BCL6 gene.

In a fifth aspect the present invention provides a method producing an engineered T cell, comprising: a) genetically engineering a T cell to enhance expression of overexpress BLIMP1 and/or (ii) to knock-out or decrease expression of BCL6; and/or treating the T cell with a BCL6 inhibitor. Without wishing to be bound by any particular theory, the present inventors believe that the enhanced cytotoxic activity of T cells engineered or treated in this way will permit more efficient production of T cells for therapeutic, especially anti-cancer, use. Among the advantages contemplated herein is a reduction in so-called IL-2 addiction of the resulting T cell population. A disadvantage of IL-2 addiction is that T cell therapy using IL-2 addicted T cells may require ongoing administration of IL-2 to the patient to whom the T cell therapy has been administered. Undesirable toxicity is associated with IL-2 treatment. Therefore, avoidance or minimisation of IL-2 addiction would be highly desirable in T cells produced for therapeutic use.

In some embodiments the method further comprises culturing the T cell under conditions suitable for expansion to provide an expanded cell population.

In some embodiments the method is performed in vitro.

In some embodiments genetically engineering the T cell is performed by CRISPR/Cas9-mediated gene editing, transcription activator-like effector nucleases (TALENs) transient downregulation using short hairpin RNA (shRNA), small interfering RNA (siRNA), microRNA (miRNA) or RNA constructs for overexpression or by introducing a nucleic acid or vector into the cell.

In some embodiments the method is for producing an engineered T cell of the first aspect of the invention.

The present invention includes the combination of the aspects and preferred features described except where such a combination is clearly impermissible or is stated to be expressly avoided. These and further aspects and embodiments of the invention are described in further detail below and with reference to the accompanying examples and figures.

### BRIEF DESCRIPTION OF THE FIGURES

FIG. 1—Tumour-reactive CD4⁺ T cell acquire cytotoxic phenotype in tumour microenvironment following lymphopenia induced expansion. (A-C) B16 tumour-bearing mice were either untreated or treated at day 8 with 0.6*10⁵ Trp-1 cells alone (Trp control) or combine with radiation (RT) and aCTLA-4 or GVAX and aCTLA-4. Details of treatment regimen in FIG. 8A (A) Tumour growth and survival of the mice (n=5 mice/group). (B) TILs were isolated at day 16 post tumour inoculation. Quantification of Tbet-expressing Trp-1 cells (n=10-11 mice/group, cumulative data of 2 independent experiments) and IFNγ-expressing cells within Trp-1 compartment (n=5-6 mice/group, cumulative data of 2 independent experiments) (C) GzmB expression: representative plots are shown and quantification of GzmB− expressing Trp-1 cells (n=13-17 mice/group, cumulative data of 4 independent experiments). (D-F) Gene array. Foxp3⁻ Trp-1 cells were sorted from B16 tumour of mice treated with GVAX and aCTLA-4 or RT and aCTLA-4. (D) Experimental schema (details FIG. 8D). (E) Volcano plot comparing differential gene expression between Th Trp-1 and Th-ctx Trp-1 cells (p≤0.01) and Volcano plot comparing differential gene encoding transcription factors expression between Trp-1 Th-ctx and Trp-1 Th cells (p≤0.01) (F) Reactome pathways enrichment analysis. Shown are immune system-related pathways. Highlighted are cytokine signalling pathways (NES>2, p<0.05). Gene-set enrichment analysis of IL-2-dependent gene sets in the expression profile (Castro. 2012 GSE39110, Castro et al., 2012). (G-H) 3*10⁵ OT-II cells were transferred to B16-OVA tumour-bearing mice (experiment scheme FIG. 8F) alone or in combination with radiation and aCTLA-4 treatment. (G) Representative plots showing expression of GzmB in control and Th-ctx conditions by transgenic and endogenous CD4⁺ T cells and quantification (n=5 mice/group). (H) Representative plots showing expression of GzmB and T-bet by OT-II cells in Th-ctx condition. (I) Activated or non-activated OT-II cells were culture with B16-OVA or B16 cell for 2 h in the presence of GzmB substrate. Representative plots showing GzmB substrate-positive B16 or B16-OVA cells and quantification. Representative data of two independent experiments. All quantification plots: mean±SEM, 1-way ANOVA (*p<0.05, **p<0.01, ***p<0.001, ****p<0.0001).

FIG. 2—Blocking of IL-2 signalling in vitro decreases Gzm B expression by mouse and human CD4⁺ T cells. (A) CTV-labelled OT-II cells were cultured for 72 h in the presence of APCs with different concentration of OT-II peptide with or without 100 UI/ml IL-2. Representative plots and quantification of GzmB-expressing OT-II cells within proliferating cells (cumulative data of two independent experiments) (B) CTV-labelled OT-II cells were stimulated with 0.01 μM OVA peptide and indicated amount of cytokines. Quantification of GzmB-expressing proliferating CD4⁺ T cells shown (C) CTV-labelled OT-II cells cultures as in (A) with or without 5 μg/ml of indicated antibodies. Quantification of GzmB-expressing CD4⁺ T cells within proliferating cells compartments. (D-E) CTV-labelled murine polyclonal CD4⁺ T cells were cultured for 72 h in the presence of APC and stimulated with (D) aCD3 and indicated amount of IL-2. (E) 1 μg/ml aCD28, after 24 h post stimulation 5 μg/ml of indicated antibodies were added. Representative plots and quantification of GzmB and T-bet-expressing CD4⁺ T cells within proliferating compartment shown (representative data of two independent experiments) (F-G) CTV-labelled human polyclonal naïve CD4⁺ T cells were cultured for 96 h in the presence of autologous APC and stimulated with 1 μg/ml aCD3 and 0.5 μg/ml aCD28. (F) After 24 h ether IL-2 (100 UI/ml) or aCD25 antibody were added. Quantification of GzmB and T-bet-expressing CD4⁺ T cells within proliferating compartment shown (cumulative data of two independent experiments) (G) Indicated ratios of autologous Tregs cells added with or without IL-2. Representative plots and quantification of GzmB-expressing CD4⁺ T cells within proliferation compartments shown. All quantification plots: mean±SEM; 1-way ANOVA (*p<0.05, **p<0.01, ***p<0.001, ****p<0.0001); FIG. 2B 2-way ANOVA (*p<0.033, **p<0.0021, ***p<0.0002, ****p<0.0001).

FIG. 3—CD4 TILs in Th-ctx condition reduce Gzm B expression but retain Th1 phenotype upon IL-2 neutralization. (A-C) B16 tumour-bearing mice received Trp-1 cells at day 8 post tumour inoculation either after irradiation (Th-ctx) or without (Ctrl) and followed by aCTLA-4 and cytokine neutralizing antibodies treatment. Schema FIG. 10A. TILs and dLN cells were isolated at day 17 (A) Quantification of Ki-67− (n=7-18 mice/group) and IL-2-expressing cells (n=7-13 mice/group) within Trp-1eff compartment (B) Quantification of T-bet- and IFN□-expressing cells within Trp-1 eff compartment (n=7-13 mice/group, cumulative data of 2 independent) (C) GzmB expression in Trp-1 and endogenous CD4⁺ T cells in indicated conditions. Representative plots are shown and quantifications for LN and tumours (n=7-13 mice/group, cumulative data of 3 independent experiments). (D-E) B16-OVA tumour bearing mice received treatment from day 8 as shown in FIG. 10D. Tumour and dLN were isolated at day 18 post tumour inoculation for analysis (D) Quantification of IFNγ(n=5 mice/group) and (E) GzmB-expressing cells within OT-II compartment in indicated conditions (n=8-11 mice group, cumulative data of 2 independent experiments). All quantification plots: mean±SEM, 1-way ANOVA (*p<0.05, **p<0.01, ***p<0.001, ****p<0.0001).

FIG. 4—Increased level of available IL-2 after Treg depletion contributes to shaping T helper cells phenotype in the tumour microenvironment. (A-B) MCA205 tumour-bearing mice were treated with 100 μg of aCTLA-4 9H10 antibody on days 6, 9, and 12 after tumour implantation alone or combined with 200 μg of aIL-2, aCD8 and aMHC-II on days 6, 9, 12 and 15 after tumour implantation. (A) Growth curves of individual MCA205 tumours, showing the product of three orthogonal tumour diameters. (B) Survival of the mice in A. (C-G) MCA205 Tumour-bearing mice were treated with 100 μg of aCTLA-4, 200 μg of aIL-2 or combination of antibodies on days 6, 9, and 12 after tumour implantation. TILs and dLN were isolated at day 13 post tumour inoculation for analysis (C) Quantification of Treg cell within CD4⁺ compartment in TILs at day 13 post tumour inoculation in indicated conditions (n=10 mice/group, cumulative data of 2 independent experiments) (D) Expression of CD25 and CD122 by CD4eff TILs. Representative plots are shown with mean percentage of expression or mean fluorescent intensity and quantification indicated and quantification of CD25 and CD122-expresing cells within CD4eff compartment (n=5-10 mice/group, cumulative data of 2 independent experiments). (E) TILs and dLN isolated at day 13 from MCA205-bearing aCTLA-4 treated and untreated mice were stimulated with 50 U/ml IL-2. Representative plots of pSTAT5 expression in CD4⁺ T cells shown and quantification of pSTAT5-expressing CD4eff T cells (n=5 mice/group, representative data of 2 independent experiments) (F) Expression of indicated molecules by CD4eff and CD8 TILs. Representative plots are shown with mean percentage of expression or mean fluorescent intensity (n=5-10 mice/group, cumulative data of 2 independent experiments) (G) Quantification of GzmB-expressing cells within CD4eff dLN and TILs and Treg TILs (n=10 mice/group, cumulative data of 2 independent experiments. (H) 10.000 dLN-infiltrating CD4⁺ T cells were cultured unstimulated or stimulated with non-pulsed-DCs or MCA205-pulsed DCs on anti-GzmB-coated ELISPOT plate for 24 h. Numbers represents GzmB spots per 100.000 responding CD4⁺ T cells. Graphical representation and quantification. All quantification plots: mean±SEM, 1-way ANOVA (*p<0.05, **p<0.01, ***p<0.001, ****p<0.0001).

FIG. 5—Treg depletion without CTLA-4 blocking is sufficient to drive GzmB expression by CD4⁺ T cells. (A-E) MCA205-bearing Foxp3-DTR mice were treated with DT alone or with combination with IL-2 (schema FIG. 12F) from day 6 post tumour inoculation. dLN and tumours were isolated at day 13 post tumour inoculation for analysis. (A) Schema and quantification of Treg cells within CD4 compartments in dLN and TILs n=10 mice/group, cumulative data of 2 independent experiments). (B) Expression of Ki67 by CD4eff, representative plots are shown (C) Expression of T-bet by CD4eff, representative plots are shown. (D) Quantification of IFNγ and GM-CSF-expressing cells within CD4eff TILs (n=10 mice/group, cumulative data of 2 independent experiments). (E) Quantification of GzmB-expressing cells within CD4eff dLN and TILs (n=10 mice/group, cumulative data of 2 independent experiments). (F) MCA205-bearing Foxp3-DTR mice were treated with DT and CD4 T cells were isolated at different time post last DT treatment. Quantification of Foxp3⁺ and GzmB⁺ cells within CD4 T cell compartments at indicated time points (n=4 mice/group). Quantification of GzmB-expressing CD4⁺ T cells in relation to frequency of Treg in the LN (n=16 mice). Fit curve-one phase exponential decay, R square=0.8865. All quantification plots: mean±SEM, 1-way ANOVA (*p<0.05, **p<0.01, ***p<0.001, ****p<0.0001).

FIG. 6—T-bet is not required for CTLA-4 mediated rejection of MCA205 sarcoma. (A-C) WT and T-bet KO MCA205 tumour-bearing mice were treated with 100 μg of aCTLA-4, 200 μg of aIL-2 or combination of antibodies on days 6, 9, and 12 after tumour implantation. TILs and dLN were isolated at day 13 post tumour inoculation for analysis (A) Quantification of Treg cells (n=7-9 mice/group, cumulative data of 2 independent experiments) and IFNγ (n=4-5 mice/group) within CD4⁺ TILs and (B) Quantification of GzmB-expressing cells within CD4eff and CD8⁺ TILs compartments (n=7-9 mice/group, cumulative data of 2 independent experiments). (C) Representative plots of T-bet and Eomes expression by GzmB⁺ CD4eff and CD8⁺ TILs and quantification (n=7-9 mice/group, cumulative data of 2 independent experiments) (D) MCA205 WT and T-bet KO tumour-bearing mice were treated with 100 μg of aCTLA-4 antibody on days 6, 9, and 12 after tumour implantation alone or combined with 200 μg of aCD8 or aCD4 antibodies on days 1, 3, 8 and 17 after tumour implantation. Growth curves of individual MCA205 tumours, showing the product of three orthogonal tumour diameters and Survival of the mice. (E) RT-qPCR for indicated transcription factors in purified MCA205 CD4 Fopx3-TILS and LN at day 12 post tumour inoculation (untreated vs. aCTLA-4 treated mice) Results shown are expression values relative to HPRT using the 2^{-ΔΔC(t)} method, (n=6 mice/condition). All quantification plots: mean±SEM, 1-way ANOVA (*p<0.05, **p<0.01, ***p<0.001, ****p<0.0001).

FIG. 7—IL-2 milieu in tumour controls differentiation of cytotoxic CD4⁺ T cells in Blimp-1-dependent manner. (A) CTV-labelled MACS-sorted CD4⁺ T cells from WT and Blimp-1 CKO mice were cultured for 72 h in the presence of APCs with 0.1 μg/ml anti-CD3 antibody and indicated amount of IL-2. Representative plots and quantification of GzmB expressing CD4⁺ T cells within proliferating cells and GzmB-APC MFI (n=3, representative data of 2 independent experiments). (B-E) Purified CD4⁺ T cells from WT and Blimp-1 CKO mice transduced with Trp-1 expressing vector and transferred at day 8 to B16-bearing mice alone or in combination with aCTLA-4 treatment and radiation (B) Schema (C-D) t-sne maps displaying cells from the indicating conditions and coloured by the main populations based on manual annotation of PhenoGraph clustering. Quantification of fractions of the cells in concatenated clusters (D) representative t-sne maps showing heat-map expression of indicated markers. (E) Representative plots and quantification of GzmB- and IFNγ-expressing cells within Trp-1 effector TILs (n=6 mice/group, representative data of two independent experiments). Analysis based on manual gating. (F-G) Blimp-1^{fl/fl} and Blimp-1 CKO MCA205 tumour-bearing mice were treated with 100 μg of aCTLA-4 9H10 on days 6, 9, and 12 after tumour implantation. TILs and dLN were isolated at day 12 post tumour inoculation for analysis (F) Quantification of Gzm B and T-bet-expressing cells within CD4eff TILs (n=9-11 mice/group, cumulative data of 2 independent experiments). (G) TILs and dLN cells were stimulated with 50 IU/ml IL-2. Representative histograms of pSTAT5 expression in LN Treg and quantification of pSTAT5-expressing Tregs cells (n=9-11 mice/group, cumulative data of 2 independent experiments) and CD25- and pSTAT5 expressing CD4eff TILs (n=5). (H) Blimp-1^{fl/f1} and Blimp-1 CKO MCA205 tumour-bearing mice were treated with 100 μg of aCTLA-4 9H10 on days 6, 9, and 12 after tumour implantation. Shown are tumour growth and survival of the mice. All quantification plots: mean±SEM, 1-way ANOVA (*p<0.05, **p<0.01, ***p<0.001, ****p<0.0001).

FIG. 8—(A-C) B16 tumour-bearing mice were treated at day 8 with 0.6*10⁵ Trp-1 cells alone or combine with aCTLA-4, radiation or GVAX (A) Experimental schema FIG. 1A (B) TILs were isolated at day 16 post tumour inoculation. Quantificatiion of Ki67-expressing Trp-1 cells, number or Trp-1 effector cells and Trp-1 effector to all Treg ratio in tumour (n=10-11 mice/group, cumulative data of 2 independent experiments). (C) Quantification of and IL-2 expressing cells within Trp-1 compartment (n=5-6 mice/group) (D-E) Transcriptome analysis (D) Experimental schema FIG. 1D-F (E) Relative expression of T-bet and Eomes genes in Trp-1 Th and Trp-1 Th-ctx condition in comparison to Trp-1 control cells (F) Reactome pathways enrichment analysis. Shown are the highest upregulated pathway in Th condition (NES>2, p<0.5). (G-H) OT-II T cells transfer to B16-OVA bearing mice (G) Experimental schema FIG. 1G (H) Tumour growth 11 quanQficaQon plots: mean±SEM, 1-way ANOVA (* p<0.05, **p<0.01, ***p<0.001, ****p<0.0001).

FIG. 9—(A) CTV-labelled Trp-1 cells were cultured for 72 h with APC and indicated amount of peptide either with 100 UI/ml IL-2 or 5 μg/ml aCD25. Quantification of GzmB and T-bet expression within proliferating compartments) (B) CTV-labelled OT-II cells cultures as in (A) with or without 5 μg/ml of indicated antibodies. Quantification of T-bet-expressing CD4 T cells within proliferating compartments. (C) Experiment as in FIG. 2G. Quantification of proliferating CD4 T cells in indicated conditions without and with addition of IL-2. All quantification plots: mean±SEM, 1-way ANOVA (*p<0.05, **p<0.01, ***p<0.001, ****p<0.0001).

FIG. 10—(A-C) B16 tumour-bearing mice received Trp-1 cells at day 8 post tumour inoculation either after irradiation or without (Cntrl) and followed by a CTLA-4 and cytokine neutralizing antibodies treatment. (A) Schema of experiment in FIG. 3 (A-C). (B) Quantification of CD25-expressing cells (C) Quantification of GM-CSF-expressing cells within Trp-1eff compartment (n=7-13 mice/group, cumulative data of 2 independent experiments). (D) B16-OVA-tumour bearing mice received OT-II cells at day 8 post tumour inoculation alone or in combination with RT and cytokine neutralizing antibodies. Schema of experiment in FIG. 3D.

FIG. 11—(A) Tumour growth, experiment as in FIG. 4C-G (B) MFI of pSTAT5 in CD4 effector cells in FIG. 4E (n=5 mice/group, representative data of 2 independent experiments) (C) MCA205 TILs: Quantification of indicated markers within CD4eff compartment as in FIG. 4F (D) Quantification of T-bet-expressing cells within CD4+ T cells (E) Quantification of GzmB-expressing cells within CD8 T and NK1.1 cells compartment (n=10 mice/group, cumulative data of 2 independent experiments) (E) 800 CD4+T ILs cells were cultured unstimulated or stimulated with non-pulsed DCs or MCA205-pulsed DCs on anti-Gzm-B-coated ELISPOT plate for 24 h. Numbers represents Gzm-B spots per 800 responding CD4+ T cells. All quantification plots: mean±SEM, 1-way ANOVA (*p<0.05, **p<0.01, ***p<0.001, ****p<0.0001).

FIG. 12—(A) MCA205 tumour-bearing mice were treated from d6 with DT at days 6, 7, 8, 9 and 10 and aIL-2 (200 ug) at days 6, 8, 10 and 12 (A) Schema of experiment as in FIG. 5A-E (B) Quantification of Ki67− expressing CD4+ T cells in dLN and TILs (C) T-bet-expressing CD4+ T cells in dLN and TILs (D) Quantification of GzmB− expressing CD8+ T cells in dLN and TILs (n=10 mice/group, cumulative data of 2 independent experiments). (E) MCA205-bearing Foxp3-DTR mice were treated with DT and CD4+ T cells were isolated at different time post last DT treatment. Quantification of T-bet+ cells within CD4 compartments at indicated Dme points (n=4 mice/group). All quantification plots: mean±SEM, 1-way ANOVA (* p<0.05, **p<0.01, ***p<0.001, ****p<0.0001).

FIG. 13—(A-E) Experiment as in FIG. 7B-E (A) Schema of experiment in FIG. 7B. (B) B16 tumour growth as in FIG. 7B. (C) Heatmap representation of Phenograph cluster. (D) Expression of markers that are not differentially expressed between clusters 1-2 and 3-5. As control expression in endogenous CD4 T cells shown in grey. (E) Quantification of T-bet, TNFa, IL-2, CD69, Lag3, OX-40-expressing cells (based on manual gating) within Trp-1 compartment in indicated conditions. (F-G) Experiments as in FIG. 7F-G (F) Quantification of GzmB within Treg TILs compartments. (G) Quantification of CD25-expressing Treg cells in LN and pSTAT5±cells within CD4eff LN compartments. All quantification plots: mean±SEM, 1-way ANOVA (*p<0.05, **p<0.01, ***p<0.001, ****p<0.0001).

FIG. 14 Percentage of CD4⁺ effector T cells that express GZMB, BCL6 and PD-1 following treatment with 10 nM of Bcl6 degrader BI-3802. Cells were activated with low dose αCD3+αCD28 (0.1 ug/ml) and cultured for 72 hrs with a control compound (BI-5273) or the BCL6 degrader BI-3802. A significantly greater percentage of CD4⁺ T Cells treated with the BCL6 targeting drug express GZMB relative to cells treated with a control compound.

FIG. 15 Mean fluorescence intensity of GZMB and BCL6 expressed in CD4⁺ effector T cells following treatment with 10 nM of Bcl6 degrader BI-3802. Cells were activated with low dose αCD3+αCD28 (0.1 ug/ml) and cultured for 72 hrs with a control compound (BI-5273) or the BCL6 degrader BI-3802. Cells treated with the BCL6 targeting drug have increased GZMB expression compared to cells treated with a control compound.

FIG. 16 Percentage of human CD4⁺ effector T cells that express GZMB and BCL6 following treatment with 100 nM of BCL6 degrader CCT369260. Cells were activated with low dose αCD3 (0.5 ug/ml) and cultured for 72 hrs with a control compound (BI-5273) or the BCL6 degrader CCT369260. A significantly greater percentage of CD4⁺ T Cells treated with the BCL6 targeting drug express GZMB relative to cells treated with a control compound.

FIG. 17 Percentage of human CD4⁺ effector T cells that express GZMB and BCL6 following treatment with 100 nM of BCL6 degrader COMPOUND X. Cells were activated with low dose αCD3 (0.5 ug/ml) and cultured for 72 hrs with a control compound (BI-5273) or the BCL6 degrader COMPOUND X. A significantly greater percentage of CD4⁺ T Cells treated with the BCL6 targeting drug express GZMB relative to cells treated with a control compound.

FIG. 18 Mean fluorescence intensity (MFI) of GZMB and BCL6 expressed in CD4⁺ effector T cells following treatment with BCL6 degrader compounds. Cells were activated with low dose αCD3 (0.5 ug/ml) and cultured for 72 hrs with 100 nM of a control compound (BI-5273) or the BCL6 degrader compounds CCT369260 and COMPOUND X. Cells treated with the BCL6 targeting drugs have increased GZMB expression compared to cells treated with the control compound.

FIG. 19 Percentage of human CD8⁺ T cells that express GZMB and BCL6 following treatment with 100 nM of BCL6 degrader CCT369260. Cells were activated with low dose αCD3 (0.5 ug/ml) and cultured for 72 hrs with a control compound (BI-5273) or the BCL6 degrader. A significantly lower percentage of CD8⁺ T Cells treated with the BCL6 targeting drug express BCL6 as compared to the cells treated with control compound.

FIG. 20 Percentage of human CD8⁺ T cells that express GZMB and BCL6 following treatment with 100 nM of BCL6 degrader COMPOUND X. Cells were activated with low dose αCD3 (0.5 ug/ml) and cultured for 72 hrs with a control compound (BI-5273) or the BCL6 degrader. A significantly lower percentage of CD8⁺ T Cells treated with the BCL6 targeting drug express BCL6 compared to cells treated with the control compound, which is accompanied by a significant increase in the percentage of CD8⁺ T cells which express GZMB.

FIG. 21 Development of a Bcl6 conditional KO, showing exons 7, 8 and 9 flanked by loxP sites. When CD4Cre is present, T cells lack Bcl6.

FIG. 22 Schematic of experimental setup, using CD4Cre (Ctrl) and CD4Cre Bcl6^{fl/f1} mice. Mice are injected subcutaneously with 0.5×10⁶ MCA205 tumour cells and treated with aCTLA4 or aCTLA4+ aIL2 on days 6, 9 and 11 after tumour inoculation.

FIG. 23 Representative flow plots showing GzmB expression by CD4⁺ Teff cells from tumours and draining LN of CD4Cre mice.

FIG. 24 Representative flow plots showing GzmB expression by CD4⁺ Teff cells from tumours and draining LN of CD4Cre Bcl6^{fl/f1} mice.

FIG. 25 Percentage of CD4⁺ Teff, Treg and CD8⁺ T cells that express GzmB in MCA205 tumours and draining LNs. Mice lacking Bcl6 in their T cell compartment show a significant increase in the percentage of GzmB⁺ cells when treated with αCTLA4+αIL2 when compared to control mice, but not in aCTLA4 treated conditions, where GzmB expression is already high. Data combined from 2 independent experiments, n=6-10 mice per group.

FIG. 26 Representative flow plots showing GzmB expression by CD4⁺ Teff cells from tumours and draining LN of CD4Cre mice and CD4Cre Bcl6^{fl/f1} mice.

FIG. 27 Quantification of the flow plot of FIG. 26. It is apparent that Bcl6 knock-out mice displayed higher percentage of GzmB positive CD4⁺ T cells.

FIG. 28 BCL6 Knock-out T cells show increased production of GZMB following in vitro restimulation. Human peripheral blood mononuclear cells, were stimulated for three days using αCD3 and αCD28 antibodies. On day three, cells were electroporated with the Cas9 protein and with the crRNA targeting BCL6. Cells were kept in culture for 10 days using low doses of interleukin 2. On day 10 cells were stained with cell trace violet and restimulated for four days with a low dose of dynabeads containing αCD3 and αCD28. On day 12, cells were incubated with brefeldin A for four hours in order to accumulate cytokines. Cells were stained for flow cytometry and acquired in the FACS symphony. Left panels show representative panels of GZMB versus PD1 in both CD4 (upper) and CD8 T cells (lower) for control cells. Right panels show representative panels of GZMB versus PD1 in both CD4 (upper) and CD8 T cells (lower) for BCL6 knock-out cells.

FIG. 29 Quantification of the boxed cells from FIG. 28. The Percentage of GZMB positive cells is plotted for Control CD4 T cells (left-most bar), BCL6 KO T cells (second bar), Control CD8 T cells (third bar) and BCL6 KO T cells (right-most bar). It is clear that BCL6 knock-out caused an increase in the percentage of GZMB positive T cells in both the CD4⁺ and CD8⁺ compartments.

Table 1—List of differentially expressed genes between Th Trp-1 and Th-ctx Trp-1 cells (p<0.01 and FC≥2).

Table 2—List of differentially expressed transcription factors between Th Trp-1 and Th-ctx Trp-1 cells (p<0.01 and FC≥2).

TABLE 3
qPCR primers. The sequences shown in Table 3 are:

| | | | SEQ ID |
|---|---|---|---|
| Target | Forward | Reverse | NO: |
| Hprt1 | TCAGTCAACGGGGGACAT | GGGGCTGTACTGCTTAAC | 2 |
| | AAA | CAG | |
| GzmB | CTGGGTCTTCTCCTGTTC | GACCCTACATGGCCTTAC | 3 |
| | TTTG | TTTC | |
| Prdm1 | GAAGGGAACACGCTTTGG | GATTCACGTAGCGCATCC | 4 |
| | AC | AG | |
| Tbx1 | CCAGCACCAGACAGAGAT | TCCACCAAGACCACATCC | 5 |
| | GA | AC | |
| Bcl-6 | CCTGTGAAATCTGTGGCA | CGCAGTTGGCTTTTGTGA | 6 |
| | CTCG | C | |
| Tox | CCATGGACCTGCCAGAGA | TTCTGCGTTCCCAATCTC | 7 |
| | TC | TTG | |

### DETAILED DESCRIPTION OF THE INVENTION

In describing the present invention, the following terms will be employed, and are intended to be defined as indicated below.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

BCL6

“BCL6” as used herein refers to B-cell lymphoma 6 protein encoded by the gene BCL6. The UniProt accession number for the human BCL6 protein is P41182. The amino acid sequence of human BCL6 is shown at UniProt P41182-1, dated 1 Feb. 1995—v1 (incorporated herein by reference in its entirety).

BCL6 Inhibitors

A “BCL6 inhibitor” as used herein refers to a compound or agent (including an agent interfering with BCL6 gene expression such as RNAi) that inhibits the function of BCL6 as a transcriptional repressor.

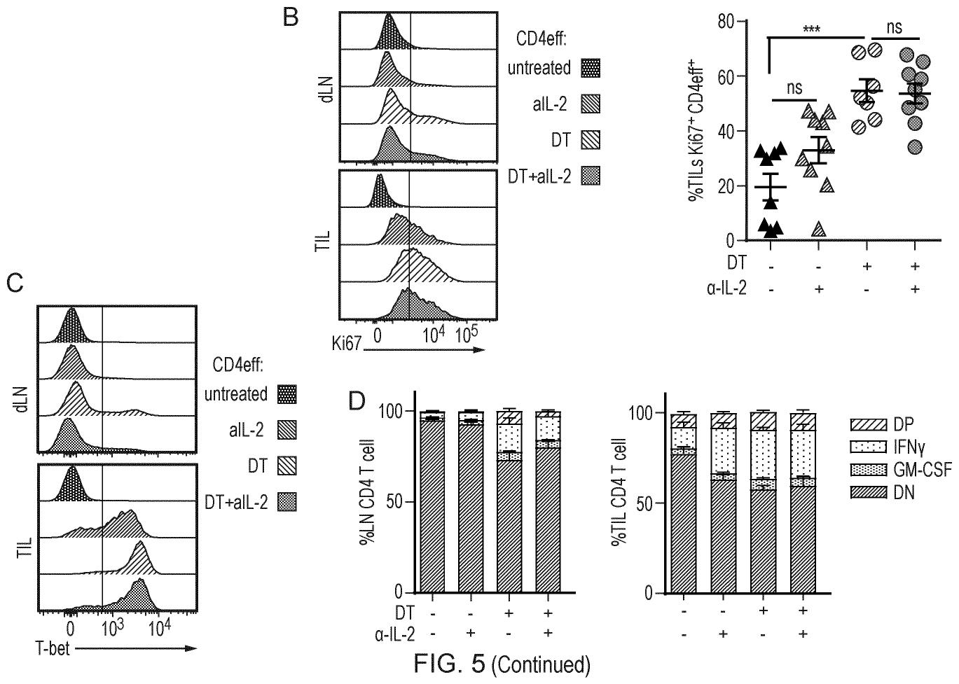

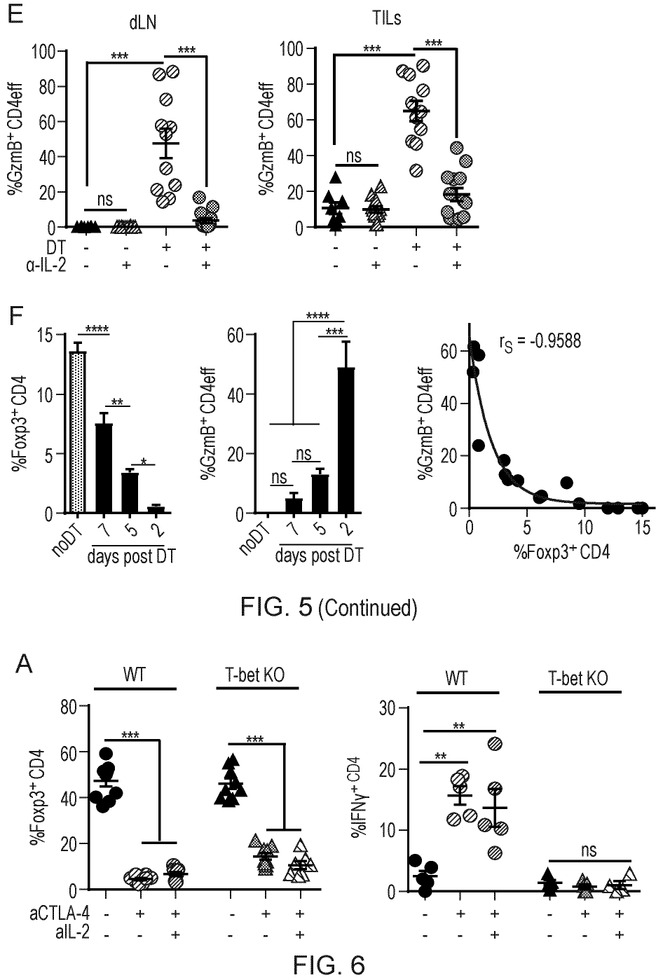

It has been found that treatment with BCL6 inhibitor compounds is able to induce similar effects as gene knock-out (KO) of BCL6. For example, in Schlager et al., 2020, Oncotarget 11(9), pp. 875-890 doi: 10.18632/oncotarget.27506, the authors compared the effect of BCL6 KO with the effect of BCL6 degraders directly, by RNA-seq in human diffuse large B cell lymphoma and their conclusion was that the effects were comparable: see, in particular, FIG. 5 of Schlager et al. Although that study was in the context of B-cells not T cells, this nevertheless provides support for the transferability of BCL6 gene KO experimental data to expected effects of pharmacological inhibition of BCL6. In some embodiments, the BCL6 inhibitor may be a small molecule or a peptide. In some embodiments the BCL6 inhibitor may be a 6-amino-quinolinone or derivative thereof as disclosed in WO2018/108704. In other embodiments the BCL6 inhibitor may be any one of the compounds 1-5 disclosed in WO2008/066887., In some embodiments the BCL6 inhibitor may be a benzimidazolone derived inhibitor disclosed in WO2018/215801. In particular, the BCL6 inhibitor may be CCT369260 which is disclosed in WO2018/215801 and in Bellenie, B. R.; Cheung, K.-M. J.; Varela, A.; Pierrat, O. A.; Collie, G. W.; Box, G. M.; Bright, M. D.; Gowan, S.; Hayes, A.; Rodrigues, M. J.; Shetty, K. N.; Carter, M.; Davis, O. A.; Henley, A. T.; Innocenti, P.; Johnson, L. D.; Liu, M.; de Klerk, S.; Le Bihan, Y.-V.; Lloyd, M. G.; McAndrew, P. C.; Shehu, E.; Talbot, R.; Woodward, H. L.; Burke, R.; Kirkin, V.; van Montfort, R. L. M.; Raynaud, F. I.; Rossanese, O. W.; Hoelder, S. Achieving In Vivo Target Depletion through the Discovery and Optimization of Benzimidazolone BCL6 Degraders. J. Med. Chem. 2020, 63 (8), 4047-4068. https://doi.org/10.1021/acs.jmedchem.9b02076. CCT369260 has the following structure and name:

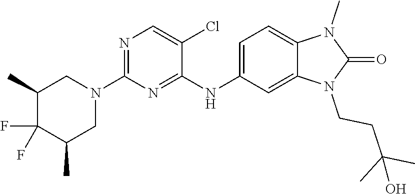 [SMILES: C[C@@H]1CN(c2ncc(Cl)c(Nc3ccc4c(c3)n(CCC(C)(C)O)c(=O)n4C)n2)C[C@H](C)C1(F)F]

### 5-((5-chloro-2-((3R,5S)-4,4-difluoro-3,5-dimethylpiperidin-1-yl)pyrimidin-4-yl)amino)-3-(3-hydroxy-3-methylbutyl)-1-methyl-1,3-dihydro-2H-benzo[d]imidazol-2-one

In another embodiment the BCL6 inhibitor may be an oxindole derived compound as disclosed in WO2014/204859, US2012/0014979, Cerchietti et al., Cancer Cell, 2010, Vol. 17(4), pp. 400-411 and Cardenas et al., J. Clin. Invest., 2016, Vol. 126(9), pp. 3351-3362 (the contents of each of which are expressly incorporated herein by reference). In another embodiment the BCL6 inhibitor may be the peptide inhibitor disclosed in WO2014/204859 and US2012/0014979.

In some embodiments the BCL6 inhibitor may be a BCL6 inhibitor compound as disclosed in WO2019/197842.

In some embodiments, the BCL6 inhibitor may be a BCL6 degrader. In Kerres et al., 2017 Cell Reports 20, pp. 2860-2875, (https://doi.org/10.1016/j.celrep.2017.08.081) the authors compared highly potent BCL6 inhibitors in diffuse large B-cell lymphoma (DLBCL) cells. They found that a subset of these inhibitors also causes rapid ubiquitylation and degradation of BCL6 in cells. These compounds display significantly stronger induction of expression of BCL6-repressed genes and anti-proliferative effects than compounds that merely inhibit co-repressor interactions. From this study there is evidence that both BCL6 degraders and inhibitors affect a similar repertoire of genes, with the degraders simply displaying stronger effect sizes than the inhibitors. It is important to stress that only BCL6-degrading compounds effectively curbed proliferation in the DLBCL study. Compounds that inhibited the binding of BCL6 to co-repressors with identical potencies, but did not cause BCL6 degradation, only had marginal effects on proliferation. Without wishing to be bound by any particular theory, the present inventors believe that BCL6 degrader compounds may be preferred for the therapeutic uses in accordance with the present invention.

In particular, the BCL6 degrader may be BI-3802 having the following chemical structure:

BI-3802

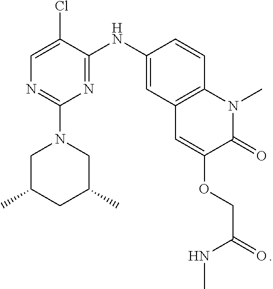 [SMILES: CNC(=O)COc1cc2cc(Nc3nc(N4C[C@H](C)C[C@H](C)C4)ncc3Cl)ccc2n(C)c1=O]

However, in some embodiments it is also contemplated that the BCL6 inhibitor may be a compound such as BI-3812 that is not a BCL6 degrader.

BLIMP-1

“BLIMP-1” (also known as PR domain zinc finger protein 1 or PRDM1 is encoded by the gene PRDM1. The UniProt accession number for the human BLIMP-1 is 075626. The amino acid sequence of human BLIMP-1 is shown at 075626-1, dated 18 May 2010—v2 (incorporated herein by reference in its entirety)

Chimeric Antigen Receptors

Chimeric Antigen Receptors (CARs) are recombinant receptor molecules which provide both antigen-binding and T cell activating functions. CAR structure and engineering is reviewed, for example, in Dotti et al., Immunol Rev (2014) 257(1), which is hereby incorporated by reference in its entirety.

CARs comprise an antigen-binding domain linked to a transmembrane domain and a signalling domain. An optional hinge domain may provide separation between the antigen-binding domain and transmembrane domain, and may act as a flexible linker.

The antigen-binding domain of a CAR may be based on the antigen-binding region of an antibody which is specific for the antigen to which the CAR is targeted. For example, the antigen-binding domain of a CAR may comprise amino acid sequences for the complementarity-determining regions (CDRs) of an antibody which binds specifically to the target protein. The antigen-binding domain of a CAR may comprise or consist of the light chain and heavy chain variable region amino acid sequences of an antibody which binds specifically to the target protein. The antigen-binding domain may be p7rovided as a single chain variable fragment (scFv) comprising the sequences of the light chain and heavy chain variable region amino acid sequences of an antibody. Antigen-binding domains of CARs may target antigen based on other protein:protein interaction, such as ligand:receptor binding; for example an IL-13Rα2-targeted CAR has been developed using an antigen-binding domain based on IL-13 (see e.g. Kahlon et al. 2004 Cancer Res 64(24): 9160-9166).

The transmembrane domain is provided between the antigen-binding domain and the signalling domain of the CAR. The transmembrane domain provides for anchoring the CAR to the cell membrane of a cell expressing a CAR, with the antigen-binding domain in the extracellular space, and signalling domain inside the cell. Transmembrane domains of CARs may be derived from transmembrane region sequences for CD3-ζ, CD4, CD8 or CD28.

The signalling domain allows for activation of the T cell. The CAR signalling domains may comprise the amino acid sequence of the intracellular domain of CD3-ζ, which provides immunoreceptor tyrosine-based activation motifs (ITAMs) for phosphorylation and activation of the CAR-expressing T cell. Signalling domains comprising sequences of other ITAM-containing proteins have also been employed in CARs, such as domains comprising the ITAM containing region of FcγRI (Haynes et al., 2001 J Immunol 166(1):182-187). CARs comprising a signalling domain derived from the intracellular domain of CD3-ζ are often referred to as first generation CARs.

Signalling domains of CARs may also comprise co-stimulatory sequences derived from the signalling domains of co-stimulatory molecules, to facilitate activation of CAR-expressing T cells upon binding to the target protein. Suitable co-stimulatory molecules include CD28, OX40, 4-1BB, ICOS and CD27. CARs having a signalling domain including additional co-stimulatory sequences are often referred to as second generation CARs.

In some cases CARs are engineered to provide for co-stimulation of different intracellular signalling pathways. For example, signalling associated with CD28 costimulation preferentially activates the phosphatidylinositol 3-kinase (P13K) pathway, whereas the 4-1BB-mediated signalling is through TNF receptor associated factor (TRAF) adaptor proteins. Signalling domains of CARs therefore sometimes contain co-stimulatory sequences derived from signalling domains of more than one co-stimulatory molecule. CARs comprising a signalling domain with multiple co-stimulatory sequences are often referred to as third generation CARs.

An optional hinge region may provide separation between the antigen-binding domain and the transmembrane domain, and may act as a flexible linker. Hinge regions may be flexible domains allowing the binding moiety to orient in different directions. Hinge regions may be derived from IgG1 or the CH₂CH₃ region of immunoglobulin.

Neoantigen Reactive T Cells (NAR-T)

A neoantigen is a newly formed antigen that has not been previously presented to the immune system. The neoantigen is tumour-specific, which arises as a consequence of a mutation within a cancer cell and is therefore not expressed by healthy (i.e. non-tumour) cells.

The neoantigen may be caused by any non-silent mutation which alters a protein expressed by a cancer cell compared to the non-mutated protein expressed by a wild-type, healthy cell. For example, the mutated protein may be a translocation or fusion.

A “mutation” refers to a difference in a nucleotide sequence (e.g. DNA or RNA) in a tumour cell compared to a healthy cell from the same individual. The difference in the nucleotide sequence can result in the expression of a protein which is not expressed by a healthy cell from the same individual. For example, the mutation may be a single nucleotide variant (SNV), multiple nucleotide variants, a deletion mutation, an insertion mutation, a translocation, a missense mutation or a splice site mutation resulting in a change in the amino acid sequence (coding mutation).

The human leukocyte antigen (HLA) system is a gene complex encoding the major histocompatibility complex (MHC) proteins in humans. A neoantigen may be processed to generate distinct peptides which can be recognised by T cells when presented in the context of MHC molecules. A neoantigen presented as such may represent a target for therapeutic or prophylactic intervention in the treatment or prevention of cancer in a subject.

An intervention may comprise an active immunotherapy approach, such as administering an immunogenic composition or vaccine comprising a neoantigen to a subject. Alternatively, a passive immunotherapy approach may be taken, for example adoptive T cell transfer or B cell transfer, wherein a T and/or B cells which recognise a neoantigen are isolated from tumours, or other bodily tissues (including but not limited to lymph node, blood or ascites), expanded ex vivo or in vitro and readministered to a subject.

T cells may be expanded by ex vivo culture in conditions which are known to provide mitogenic stimuli for T cells. By way of example, the T cells may be cultured with cytokines such as IL-2 or with mitogenic antibodies such as anti-CD3 and/or CD28. The T cells may be co-cultured with antigen-presenting cells (APCs), which may have been irradiated. The APCs may be dendritic cells or B cells. The dendritic cells may have been pulsed with peptides containing the identified neoantigen as single stimulants or as pools of stimulating neoantigen peptides. Expansion of T cells may be performed using methods which are known in the art, including for example the use of artificial antigen presenting cells (aAPCs), which provide additional co-stimulatory signals, and autologous PBMCs which present appropriate peptides. Autologous PBMCs may be pulsed with peptides containing neoantigens as single stimulants, or alternatively as pools of stimulating neoantigens.

Engineered T Cell

The present invention provides an engineered T cell in which the BCL6/BLIMP-1 balance has been modulated so as to enhance cytotoxic activity.

The cell may be a eukaryotic cell, e.g. a mammalian cell. The mammal may be a human, or a non-human mammal (e.g. rabbit, guinea pig, rat, mouse or other rodent (including any animal in the order Rodentia), cat, dog, pig, sheep, goat, cattle (including cows, e.g. dairy cows, or any animal in the order Bos), horse (including any animal in the order Equidae), donkey, and non-human primate).

In some embodiments, the cell may be from, or may have been obtained from, a human subject.

The cell is preferably a CD4⁺ T cell. In some embodiments, the cell is a target protein-reactive CAR-T cell. In embodiments herein, a “target protein-reactive” CAR-T cell is a cell which displays certain functional properties of a T cell in response to the target protein for which the antigen-binding domain of the CAR is specific, e.g. expressed at the surface of a cell. In some embodiments, the properties are functional properties associated with effector T cells, e.g. cytotoxic T cells.

In some embodiments, the engineered T cell may display one or more of the following properties: cytotoxicity to a cell comprising or expressing the target protein; proliferation, increased IFNγ expression, increased CD107a expression, increased IL-2 expression, increased TNFα expression, increased perforin expression, increased granzyme B expression, increased granulysin expression, and/or increased FAS ligand (FASL) expression in response to the target protein, or a cell comprising or expressing the target protein.

Gene expression can be measured by a various means known to those skilled in the art, for example by measuring levels of mRNA by quantitative real-time PCR (qRT-PCR), or by reporter-based methods. Similarly, protein expression can be measured by various methods well known in the art, e.g. by antibody-based methods, for example by western blot, immunohistochemistry, immunocytochemistry, flow cytometry, ELISA, ELISPOT, or reporter-based methods.

The present invention also provides a method for producing an engineered T cell according to the present invention, comprising genetically engineering a T cell (e.g. by CRISPR/Cas9-mediated gene editing, transcription activator-like effector nucleases (TALENs) transient downregulation using short hairpin RNA (shRNA), small interfering RNA (siRNA), microRNA (miRNA) or RNA constructs for overexpression or by introducing a nucleic acid or vector into the cell) to enhance expression of BLIMP-1 and/or knock-out or downregulate expression of BCL6. In some embodiments, the methods additionally comprise culturing the T cell under conditions suitable for expansion to provide an expanded cell population. In some embodiments, the methods are performed in vitro.

In some embodiments, the engineered T cell further comprises an introduced T cell receptor (e.g. a chimeric antigen receptor) that specifically recognises an antigen expressed on or in proximity to a tumour (e.g. tumour stroma). The present invention also provides methods of introducing an isolated nucleic acid or vector encoding the T cell receptor into the engineered T cell. In some embodiments the isolated nucleic acid or vector is comprised in a viral vector, or the vector is a viral vector. In some embodiments, the method comprises introducing a nucleic acid or vector according to the invention by electroporation.

Compositions

The present invention also provides compositions comprising a cell according to the invention.

Engineered T cells according to the present invention may be formulated as pharmaceutical compositions for clinical use and may comprise a pharmaceutically acceptable carrier, diluent, excipient or adjuvant.

In accordance with the present invention methods are also provided for the production of pharmaceutically useful compositions, such methods of production may comprise one or more steps selected from: isolating an engineered T cell as described herein; and/or mixing an engineered T cell as described herein with a pharmaceutically acceptable carrier, adjuvant, excipient or diluent.

Uses of and Methods of Using the CARs, Nucleic Acids, Cells and Compositions

The engineered T cells and pharmaceutical compositions according to the present invention find use in therapeutic and prophylactic methods.

The present invention also provides the use of an engineered T cell or pharmaceutical composition according to the present invention in the manufacture of a medicament for treating or preventing a disease or disorder.

The present invention also provides a method of treating or preventing a disease or disorder, comprising administering to a subject a therapeutically or prophylactically effective amount of an engineered T cell or pharmaceutical composition according to the present invention.

Administration

Administration of a BCL6 inhibitor or engineered T cell or composition according to the invention is preferably in a “therapeutically effective” or “prophylactically effective” amount, this being sufficient to show benefit to the subject. The actual amount administered, and rate and time-course of administration, will depend on the nature and severity of the disease or disorder. Prescription of treatment, e.g. decisions on dosage etc., is within the responsibility of general practitioners and other medical doctors, and typically takes account of the disease/disorder to be treated, the condition of the individual subject, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

The BCL6 inhibitors and engineered T cells, compositions and other therapeutic agents, medicaments and pharmaceutical compositions according to aspects of the present invention may be formulated for administration by a number of routes, including but not limited to, parenteral, intravenous, intra-arterial, intramuscular, subcutaneous, intradermal, intratumoral and oral. The CARs, nucleic acids, vectors, cells, composition and other therapeutic agents and therapeutic agents may be formulated in fluid or solid form. Fluid formulations may be formulated for administration by injection to a selected region of the human or animal body, or by infusion to the blood. Administration may be by injection or infusion to the blood, e.g. intravenous or intra-arterial administration.

Administration may be alone or in combination with other treatments, either simultaneously or sequentially dependent upon the condition to be treated.

In some embodiments, treatment with a BCL6 inhibitor or engineered T cell or composition of the present invention may be accompanied by other therapeutic or prophylactic intervention, e.g. chemotherapy, immunotherapy, radiotherapy, surgery, vaccination and/or hormone therapy.

Simultaneous administration refers to administration of the BCL6 inhibitor, engineered T cell or composition and therapeutic agent together, for example as a pharmaceutical composition containing both agents (combined preparation), or immediately after each other and optionally via the same route of administration, e.g. to the same artery, vein or other blood vessel. Sequential administration refers to administration of one therapeutic agent followed after a given time interval by separate administration of the other agent. It is not required that the two agents are administered by the same route, although this is the case in some embodiments. The time interval may be any time interval.

Chemotherapy and radiotherapy respectively refer to treatment of a cancer with a drug or with ionising radiation (e.g. radiotherapy using X-rays or γ-rays).

The drug may be a chemical entity, e.g. small molecule pharmaceutical, antibiotic, DNA intercalator, protein inhibitor (e.g. kinase inhibitor), or a biological agent, e.g. antibody, antibody fragment, nucleic acid or peptide aptamer, nucleic acid (e.g. DNA, RNA), peptide, polypeptide, or protein. The drug may be formulated as a pharmaceutical composition or medicament. The formulation may comprise one or more drugs (e.g. one or more active agents) together with one or more pharmaceutically acceptable diluents, excipients or carriers.

A treatment may involve administration of more than one drug. A drug may be administered alone or in combination with other treatments, either simultaneously or sequentially dependent upon the condition to be treated. For example, the chemotherapy may be a co-therapy involving administration of two drugs, one or more of which may be intended to treat the cancer.

The chemotherapy may be administered by one or more routes of administration, e.g. parenteral, intravenous injection, oral, subcutaneous, intradermal or intratumoral.

The chemotherapy may be administered according to a treatment regime. The treatment regime may be a pre-determined timetable, plan, scheme or schedule of chemotherapy administration which may be prepared by a physician or medical practitioner and may be tailored to suit the patient requiring treatment.

The treatment regime may indicate one or more of: the type of chemotherapy to administer to the patient; the dose of each drug or radiation; the time interval between administrations; the length of each treatment; the number and nature of any treatment holidays, if any etc. For a co-therapy a single treatment regime may be provided which indicates how each drug is to be administered.

Chemotherapeutic drugs, immunotherapies and/or biologics may be selected from: alkylating agents such as cisplatin, carboplatin, mechlorethamine, cyclophosphamide, chlorambucil, ifosfamide; purine or pyrimidine anti-metabolites such as azathiopurine or mercaptopurine; alkaloids and terpenoids, such as vinca alkaloids (e.g. vincristine, vinblastine, vinorelbine, vindesine), podophyllotoxin, etoposide, teniposide, taxanes such as paclitaxel (Taxol™), docetaxel; topoisomerase inhibitors such as the type I topoisomerase inhibitors camptothecins irinotecan and topotecan, or the type II topoisomerase inhibitors amsacrine, etoposide, etoposide phosphate, teniposide; antitumor antibiotics (e.g. anthracyline antibiotics) such as dactinomycin, doxorubicin (Adriamycin™), epirubicin, bleomycin, rapamycin; antibody based agents, such as anti-PD-1 antibodies, anti-PD-L1 antibodies, anti-TIM-3 antibodies, anti-CTLA-4, anti-4-1BB, anti-GITR, anti-CD27, anti-BLTA, anti-OX43, anti-VEGF, anti-TNFα, anti-IL-2, antiGpIIb/IIIa, anti-CD-52, anti-CD20, anti-RSV, anti-HER2/neu(erbB2), anti-TNF receptor, anti-EGFR antibodies, monoclonal antibodies or antibody fragments, examples include: cetuximab, panitumumab, infliximab, basiliximab, bevacizumab (Avastin®), abciximab, daclizumab, gemtuzumab, alemtuzumab, rituximab (Mabthera®), palivizumab, trastuzumab, etanercept, adalimumab, nimotuzumab; EGFR inhibitors such as erlotinib, cetuximab and gefitinib; anti-angiogenic agents such as bevacizumab (Avastin®); cancer vaccines such as Sipuleucel-T (Provenge®).

Further chemotherapeutic drugs may be selected from: 13-cis-Retinoic Acid, 2-Chlorodeoxyadenosine, 5-Azacitidine 5-Fluorouracil, 6-Mercaptopurine, 6-Thioguanine, Abraxane, Accutane®, Actinomycin-D Adriamycin®, Adrucil®, Afinitor®, Agrylin®, Ala-Cort®, Aldesleukin, Alemtuzumab, ALIMTA, Alitretinoin, Alkaban-AQ®, Alkeran®, All-transretinoic Acid, Alpha Interferon, Altretamine, Amethopterin, Amifostine, Aminoglutethimide, Anagrelide, Anandron®, Anastrozole, Arabinosylcytosine, Aranesp®, Aredia®, Arimidex®, Aromasin®, Arranon®, Arsenic Trioxide, Asparaginase, ATRA Avastin®, Azacitidine, BCG, BCNU, Bendamustine, Bevacizumab, Bexarotene, BEXXAR®, Bicalutamide, BiCNU, Blenoxane®, Bleomycin, Bortezomib, Busulfan, Busulfex®, Calcium Leucovorin, Campath®, Camptosar®, Camptothecin-11, Capecitabine, Carac™, Carboplatin, Carmustine, Casodex®, CC-5013, CCI-779, CCNU, CDDP, CeeNU, Cerubidine®, Cetuximab, Chlorambucil, Cisplatin, Citrovorum Factor, Cladribine, Cortisone, Cosmegen®, CPT-11, Cyclophosphamide, Cytadren®, Cytarabine Cytosar-U®, Cytoxan®, Dacogen, Dactinomycin, Darbepoetin Alfa, Dasatinib, Daunomycin, Daunorubicin, Daunorubicin Hydrochloride, Daunorubicin Liposomal, DaunoXome®, Decadron, Decitabine, Delta-Cortef®, Deltasone®, Denileukin, Diftitox, DepoCyt™, Dexamethasone, Dexamethasone Acetate, Dexamethasone Sodium Phosphate, Dexasone, Dexrazoxane, DHAD, DIC, Diodex, Docetaxel, Doxil®, Doxorubicin, Doxorubicin Liposomal, Droxia™, DTIC, DTIC-Dome®, Duralone®, Eligard™, Ellence™, Eloxatin™, Elspar®, Emcyt®, Epirubicin, Epoetin Alfa, Erbitux, Erlotinib, Erwinia L-asparaginase, Estramustine, Ethyol Etopophos®, Etoposide, Etoposide Phosphate, Eulexin®, Everolimus, Evista®, Exemestane, Faslodex®, Femara®, Filgrastim, Floxuridine, Fludara®, Fludarabine, Fluoroplex®, Fluorouracil, Fluoxymesterone, Flutamide, Folinic Acid, FUDR®, Fulvestrant, Gefitinib, Gemcitabine, Gemtuzumab ozogamicin, Gleevec™, Gliadel® Wafer, Goserelin, Granulocyte—Colony Stimulating Factor, Granulocyte Macrophage Colony Stimulating Factor, Herceptin®, Hexadrol, Hexalen®, Hexamethylmelamine, HMM, Hycamtin®, Hydrea®, Hydrocort Acetate®, Hydrocortisone, Hydrocortisone Sodium Phosphate, Hydrocortisone Sodium Succinate, Hydrocortone Phosphate, Hydroxyurea, Ibritumomab, Ibritumomab Tiuxetan, Idamycin®, Idarubicin, Ifex®, IFN-alpha, Ifosfamide, IL-11, IL-2, Imatinib mesylate, Imidazole Carboxamide, Interferon alfa, Interferon Alfa-2b (PEG Conjugate), Interleukin-2, Interleukin-11, Intron A® (interferon alfa-2b), Iressa®, Irinotecan, Isotretinoin, Ixabepilone, Ixempra™, Kidrolase, Lanacort®, Lapatinib, L-asparaginase, LCR, Lenalidomide, Letrozole, Leucovorin, Leukeran, Leukine™, Leuprolide, Leurocristine, Leustatin™, Liposomal Ara-C, Liquid Pred®, Lomustine, L-PAM, L-Sarcolysin, Lupron®, Lupron Depot®, Matulane®, Maxidex, Mechlorethamine, Mechlorethamine Hydrochloride, Medralone®, Medrol®, Megace®, Megestrol, Megestrol Acetate, Melphalan, Mercaptopurine, Mesna, Mesnex™, Methotrexate, Methotrexate Sodium, Methylprednisolone, Meticorten®, Mitomycin, Mitomycin-C, Mitoxantrone, M-Prednisol®, MTC, MTX, Mustargen®, Mustine, Mutamycin®, Myleran®, Mylocel™, Mylotarg®, Navelbine®, Nelarabine, Neosar®, Neulasta™, Neumega®, Neupogen®, Nexavar®, Nilandron®, Nilutamide, Nipent®, Nitrogen Mustard, Novaldex®, Novantrone®, Octreotide, Octreotide acetate, Oncospar®, Oncovin®, Ontak®, Onxal™, Oprevelkin, Orapred®, Orasone®, Oxaliplatin, Paclitaxel, Paclitaxel Protein-bound, Pamidronate, Panitumumab, Panretin®, Paraplatin®, Pediapred®, PEG Interferon, Pegaspargase, Pegfilgrastim, PEG-INTRON™, PEG-L-asparaginase, PEMETREXED, Pentostatin, Phenylalanine Mustard, Platinol®, Platinol-AQ®, Prednisolone, Prednisone, Prelone®, Procarbazine, PROCRIT®, Proleukin®, Prolifeprospan 20 with Carmustine Implant Purinethol®, Raloxifene, Revlimid®, Rheumatrex®, Rituxan®, Rituximab, Roferon-A® (Interferon Alfa-2a), Rubex®, Rubidomycin hydrochloride, Sandostatin® Sandostatin LAR®, Sargramostim, Solu-Cortef®, Solu-Medrol®, Sorafenib, SPRYCEL™, STI-571, Streptozocin, SU11248, Sunitinib, Sutent®, Tamoxifen, Tarceva®, Targretin®, Taxol®, Taxotere®, Temodar®, Temozolomide, Temsirolimus, Teniposide, TESPA, Thalidomide, Thalomid®, TheraCys®, Thioguanine, Thioguanine Tabloid®, Thiophosphoamide, Thioplex®, Thiotepa, TICE®, Toposar®, Topotecan, Toremifene, Torisel®, Tositumomab, Trastuzumab, Treanda®, Tretinoin, Trexall™, Trisenox®, TSPA, TYKERB®, VCR, Vectibixm, Velban®, VelcadeR, VePesid®, Vesanoid®, Viadurm, VidazaR, Vinblastine, Vinblastine Sulfate, Vincasar Pfs®, Vincristine, Vinorelbine, Vinorelbine tartrate, VLB, VM-26, Vorinostat, VP-16, Vumon®, Xeloda®, Zanosar®, Zevalinm, Zinecard®, Zoladex®, Zoledronic acid, Zolinza, Zometa®.

Cancer

In some embodiments, the disease or disorder to be treated or prevented in accordance with the present invention is a cancer.

The cancer may be any unwanted cell proliferation (or any disease manifesting itself by unwanted cell proliferation), neoplasm or tumor or increased risk of or predisposition to the unwanted cell proliferation, neoplasm or tumor. The cancer may be benign or malignant and may be primary or secondary (metastatic). A neoplasm or tumor may be any abnormal growth or proliferation of cells and may be located in any tissue. Examples of tissues include the adrenal gland, adrenal medulla, anus, appendix, bladder, blood, bone, bone marrow, bowel, brain, breast, cecum, central nervous system (including or excluding the brain) cerebellum, cervix, colon, duodenum, endometrium, epithelial cells (e.g. renal epithelia), eye, germ cells, gallbladder, oesophagus, glial cells, head and neck, heart, ileum, jejunum, kidney, lacrimal glad, larynx, liver, lung, lymph, lymph node, lymphoblast, maxilla, mediastinum, mesentery, myometrium, mouth, nasopharynx, omentum, oral cavity, ovary, pancreas, parotid gland, peripheral nervous system, peritoneum, pleura, prostate, salivary gland, sigmoid colon, skin, small intestine, soft tissues, spleen, stomach, testis, thymus, thyroid gland, tongue, tonsil, trachea, uterus, vulva, white blood cells.

Examples of cancer to treat may be selected from bladder cancer, gastric cancer, oesophageal cancer, breast cancer, colorectal cancer, cervical cancer, ovarian cancer, endometrial cancer, kidney cancer (renal cell), lung cancer (small cell, non-small cell and mesothelioma), brain cancer (gliomas, astrocytomas, glioblastomas), melanoma, lymphoma, small bowel cancers (duodenal and jejunal), leukemia, pancreatic cancer, hepatobiliary tumours, germ cell cancers, prostate cancer, head and neck cancers, thyroid cancer and sarcomas.

Tumors to be treated may be nervous or non-nervous system tumors. Nervous system tumors may originate either in the central or peripheral nervous system, e.g. glioma, medulloblastoma, meningioma, neurofibroma, ependymoma, Schwannoma, neurofibrosarcoma, astrocytoma and oligodendroglioma. Non-nervous system cancers/tumors may originate in any other non-nervous tissue, examples include melanoma, mesothelioma, lymphoma, myeloma, leukemia, Non-Hodgkin's lymphoma (NHL), Hodgkin's lymphoma, chronic myelogenous leukemia (CML), acute myeloid leukemia (AML), myelodysplastic syndrome (MDS), cutaneous T-cell lymphoma (CTCL), chronic lymphocytic leukemia (CLL), hepatoma, epidermoid carcinoma, prostate carcinoma, breast cancer, lung cancer (e.g. small cell), colon cancer, ovarian cancer, pancreatic cancer, thymic carcinoma, NSCLC, haematologic cancer and sarcoma.

The present invention is likely to be particularly useful in the context of treatment of cancers that are considered immunogenic. These include for example melanoma, Lung squamous cell carcinoma, lung adenocarcinoma, bladder cancer, small cell lung cancer, oesophagus cancer, colorectal cancer, cervical cancer, head and neck cancer, stomach cancer, endometrial cancer, and liver cancer. Indeed, all of these types of cancers have been shown to have high somatic mutation rates (e.g. more than 5 somatic mutations per megabase in Alexandrov et al.).

Adoptive Transfer

In embodiments of the present invention, a method of treatment or prophylaxis may comprise adoptive transfer of immune cells, particularly T cells. Adoptive T cell transfer generally refers to a process by which T cells are obtained from a subject, typically by drawing a blood sample from which T cells are isolated. The T cells are then typically treated or altered in some way, optionally expanded, and then administered either to the same subject or to a different subject. The treatment is typically aimed at providing a T cell population with certain desired characteristics to a subject, or increasing the frequency of T cells with such characteristics in that subject. Adoptive transfer of CAR-T cells is described, for example, in Kalos and June 2013, Immunity 39(1): 49-60, which is hereby incorporated by reference in its entirety.

In the present invention, adoptive transfer is performed with the aim of introducing, or increasing the frequency of, target protein-reactive T cells in a subject, in particular target protein-reactive CD4⁺ T cells.

In some embodiments, the subject from which the T cell is isolated is the subject administered with the modified T cell (i.e., adoptive transfer is of autologous T cells). In some embodiments, the subject from which the T cell is isolated is a different subject to the subject to which the modified T cell is administered (i.e., adoptive transfer is of allogenic T cells).

The at least one T cell modified according to the present invention can be modified according to methods well known to the skilled person. The modification may comprise nucleic acid transfer for permanent or transient expression of the transferred nucleic acid.

In some embodiments the method may comprise one or more of the following steps: taking a blood sample from a subject; isolating and/or expanding at least one T cell from the blood sample; culturing the at least one T cell in in vitro or ex vivo cell culture; engineering the at least one T cell to increase expression of BLIMP-1 and/or to knock out or downregulate expression of BCL6; optionally inserting a modified T cell receptor or CAR, or a nucleic acid, or vector encoding the modified T cell receptor or CAR; expanding the at least one engineered T cell, collecting the at least one engineered T cell; mixing the engineered T cell with an adjuvant, diluent, or carrier; administering the engineered T cell to a subject.

In embodiments according to the present invention the subject is preferably a human subject. In some embodiments, the subject to be treated according to a therapeutic or prophylactic method of the invention herein is a subject having, or at risk of developing, a disease or disorder characterised by expression or upregulated expression of the target protein. In some embodiments, the subject to be treated is a subject having, or at risk of developing, a cancer, e.g. a cancer expressing the target protein, or a cancer in which expression of the target protein is upregulated.

In some embodiments, the method additionally comprise therapeutic or prophylactic intervention for the treatment or prevention of a disease or disorder, e.g. chemotherapy, immunotherapy, radiotherapy, surgery, vaccination and/or hormone therapy. In some embodiments, the method additionally comprises therapeutic or prophylactic intervention, for the treatment or prevention of a cancer.

T Cell Therapy

T cell therapy can include adoptive T cell therapy, tumour-infiltrating lymphocyte (TIL) immunotherapy, autologous cell therapy, engineered autologous cell therapy (eACT), and allogeneic T cell transplantation.

The T cells of the immunotherapy can come from any source known in the art. For example, T cells can be differentiated in vitro from a hematopoietic stem cell population, or T cells can be obtained from a subject. T cells can be obtained from, e.g., peripheral blood mononuclear cells, bone marrow, lymph node tissue, cord blood, thymus tissue, tissue from a site of infection, ascites, pleural effusion, spleen tissue, and tumours. In addition, the T cells can be derived from one or more T cell lines available in the art. T cells can also be obtained from a unit of blood collected from a subject using any number of techniques known to the skilled artisan, such as FICOLL™ separation and/or apheresis. Additional methods of isolating T cells for a T cell therapy are disclosed in US2013/0287748, which is herein incorporated by references in its entirety.

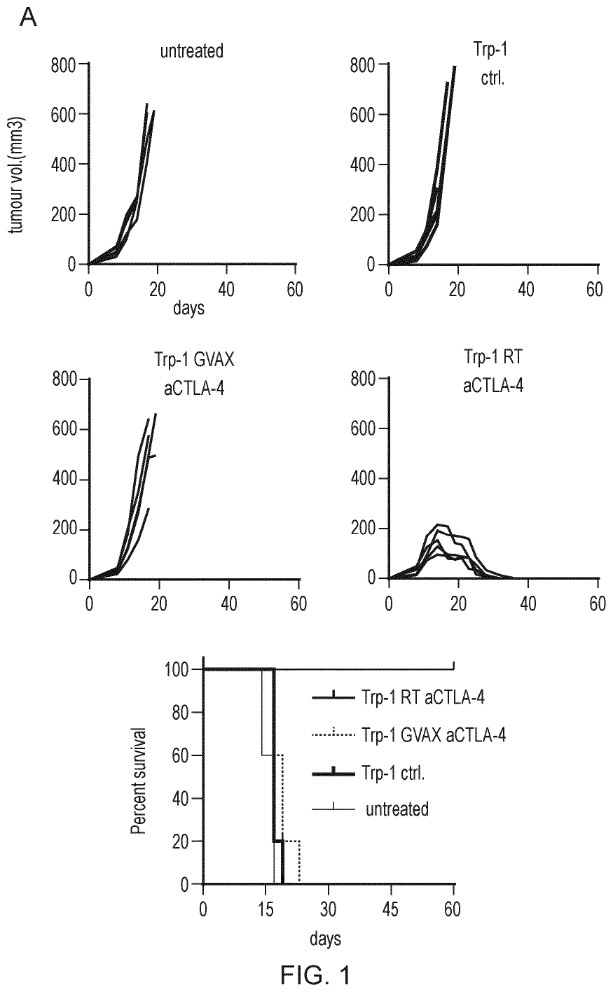

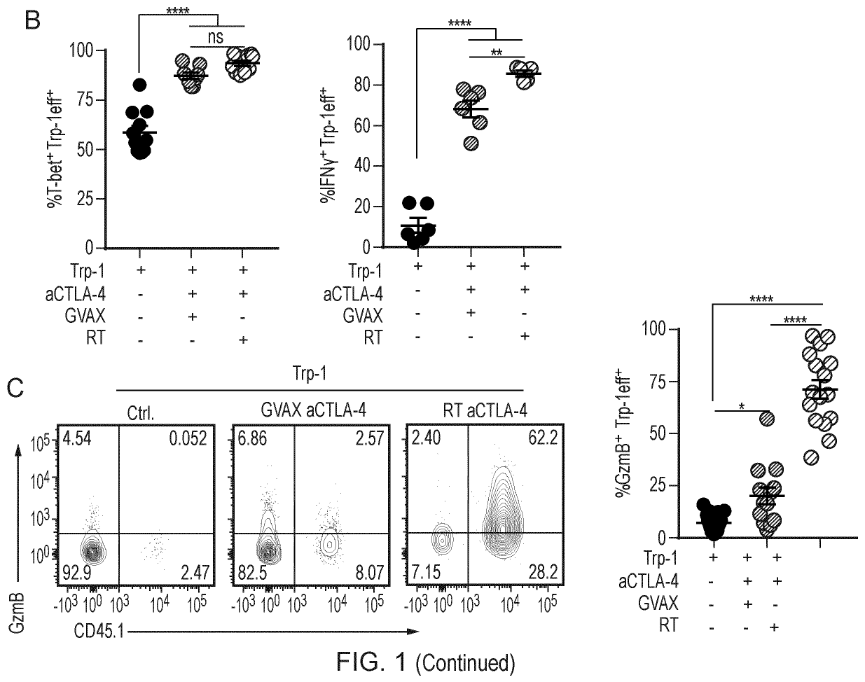

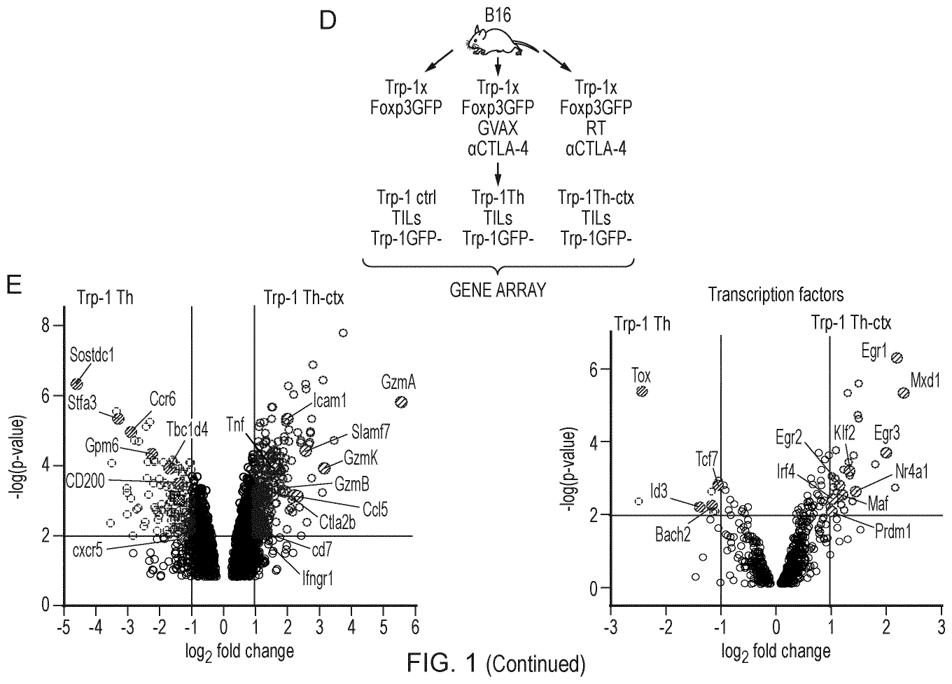

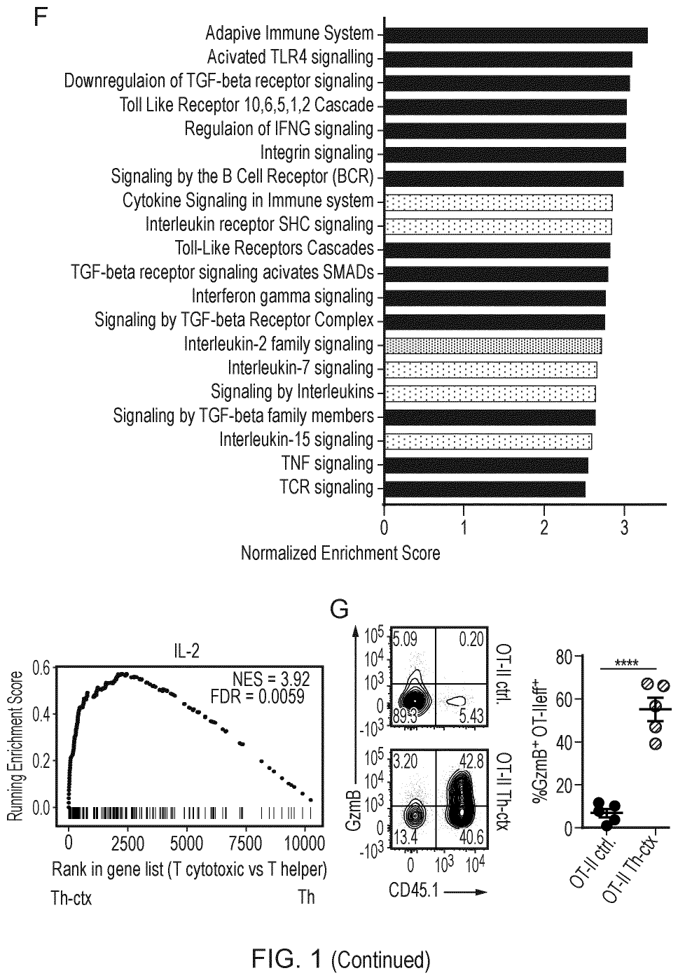

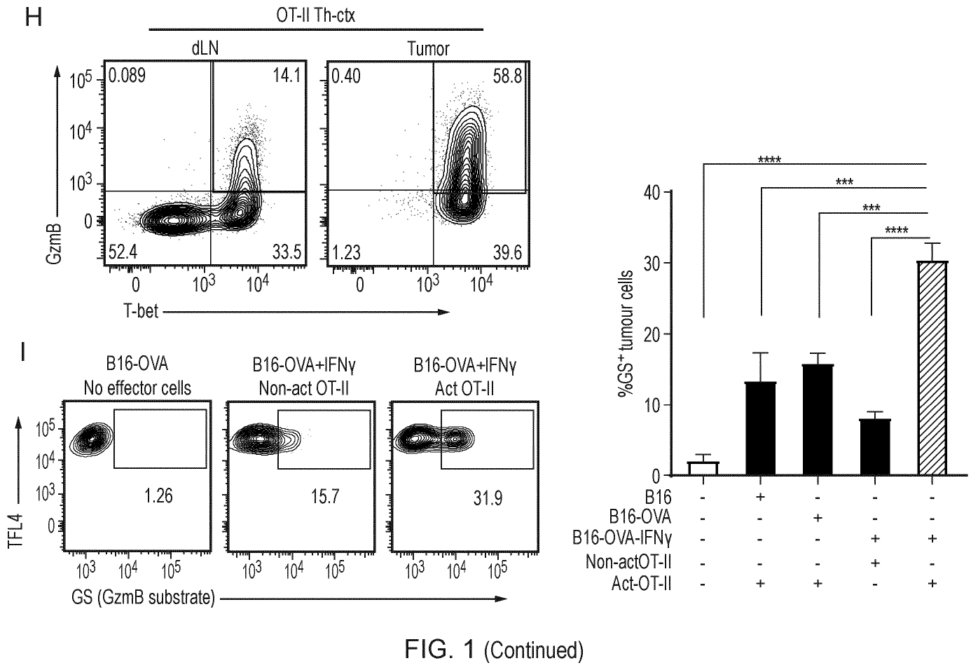

The term “engineered Autologous Cell Therapy,” which can be abbreviated as “eACT™,” also known as adoptive cell transfer, is a process by which a patient's own T cells are collected and subsequently genetically altered to recognize and target one or more antigens expressed on the cell surface of one or more specific tumour cells or malignancies. T cells can be engineered to express, for example, chimeric antigen receptors (CAR) or T cell receptor (TCR). CAR positive (+) T cells are engineered to express an extracellular single chain variable fragment (scFv) with specificity for a particular tumour antigen linked to an intracellular signalling part comprising a costimulatory domain and an activating domain. The costimulatory domain can be derived from, e.g., CD28, and the activating domain can be derived from, e.g., CD3-zeta (FIG. 1). In certain embodiments, the CAR is designed to have two, three, four, or more costimulatory domains. The CAR scFv can be designed to target, for example, CD19, which is a transmembrane protein expressed by cells in the B cell lineage, including all normal B cells and B cell malignances, including but not limited to NHL, CLL, and non-T cell ALL. Example CAR+ T cell therapies and constructs are described in US2013/0287748, US2014/0227237, US2014/0099309, and US2014/0050708, and these references are incorporated by reference in their entirety.

T cells engineered according to the present invention may be engineered at any stage before their use, in particular engineering to overexpress BLIMP1 and/or knock-out or decrease expression of BCL6 may be carried out prior to or after a step of T cell expansion.

Subjects

The subject to be treated according to the invention may be any animal or human. The subject is preferably mammalian, more preferably human. The subject may be a non-human mammal, but is more preferably human. The subject may be male or female. The subject may be a patient. A subject may have been diagnosed with a disease or condition requiring treatment, may be suspected of having such a disease or condition, or may be at risk from developing such a disease or condition.

The following is presented by way of example and is not to be construed as a limitation to the scope of the claims.

### EXAMPLES

Material and Methods

Mice

C57BL/6 and mice were purchased from Charles River Laboratories, UK. T-bet knockout mice were a kind gift from G. Lord (King's College, London, UK), CD4-Cre and B. Seddon (UCL, UK), and Blimpfl/fl mice (Shapiro-Shelef et al., 2003) from T. Korn (TUM, Munich, Germany). Trp-1 mice carry following mutation: Rag1tm1Mom×Tyrp1B-wx CD45.1+/+(Muranski et al., 2008; Quezada et al., 2010) All transgenic mice were of C57BL/6 background, bred in Charles River Laboratories (Trp-1, OT-II-CD45.1, T-bet−/−) or University College London (CD4-Cre Blimpfl/fl) animal facilities. All animal studies were performed under University College London and UK Home Office ethical approval and regulations.

Tumour Cell Lines and Tissue Culture

MCA205 tumour cells were cultured in DMEM (Sigma) supplemented with 10% fetal bovine serum (FBS, Gibco Sigma), 100 U/mL penicillin, 100 μg/mL streptomycin and 2 mM L-glutamine (all from Gibco). B16 and B16-OVA cells were cultured in RPMI media supplemented as above.

In Vivo Experiments

Mice were injected subcutaneously with 4×105 MCA205, 2.5×105 B16, 2.5×105 B16-OVA cells re-suspended in PBS. Therapeutic antibodies were administered intraperitoneally at the time points and doses shown in figure legends. Therapeutic antibodies: anti-CTLA-4 (9H10) anti-CD4 (GK1.5) and anti-CD8 (2.43), anti-IL-2 (JES-6-1A12), anti-MHC-II (M5/114), and anti-IL-7 (M25) were purchased from BioXcell. Tumours were measured at least twice weekly and mice were euthanized when any orthogonal tumour diameter reached 150 mm. Tumour volume was calculated as 4/3πabc, where a, b, and c are radii.

For adoptive cell transfer experiments melanoma-bearing mice were treated or not with 5 Gy of body irradiation, 0.6×105 Trp-1⁺ or 3×105 OT-II cells and 200 μg aCTLA-4 i.p on day 8 and 100 μg aCTLA-4 on days 11 and 14. Mice in Th conditions received irradiated 106 GVAX cells (150 Gy). Trp-1⁺ and OT-II+CD4 T cells used for adoptive transfer were isolated from naïve spleen and LN of Trp-1 and OT-II mice respectively and purified with CD4⁺ beads (130-117-043, Miltenyi) according to the manufacturer's protocol. Mice received 100 μg of aCTLA-4 antibodies on days 11 and 14. For cytokine neutralizing experiment aIL-2 or aIL-7 (200 μg) administration started at day 11 following 2 additional doses.

Functional Analysis/Tissue Processing

Mice used for functional experiments were sacrificed on day 13 (MCA205) or 17/18 (melanoma) after tumour implantation, and LN cells and tumour-infiltrating lymphocytes were isolated as previously described (Quezada et al., 2006). Tumour-infiltrating CD4+ T cells were purified using CD4 positive selection (FlowComp; Invitrogen) according to the manufacturer's instructions. Purified CD4+ T cells from tumours or bulk cells from LNs were restimulated for 4 h at 37° C. with 5×104 DCs and 1 μM of Trp1 or OVA peptide followed by addition of brefeldin A (BD) for 2 more hours. Polyfunctional CD4⁺ T cells were restimulated with phorbol 12-myristate 13-acetate (PMA, 20 ng/mL) and ionomycin (500 ng/mL; Sigma Aldrich) for 4 hours at 37° C. in the presence of GolgiPlug (BD Biosciences). For pSTAT5 staining TILs and LN cells were rested for 2 h in FCS-free media followed by 10 min stimulation with 50 IU/ml of IL-2 (Peprotech) and fixed for 30 min with Fixation/Permeabilization buffer (ThermoFisher) and Perm Buffer III (BD Phoslow) followed by the intracellular staining.

Th-Ctx and Th Trp-1 Transcriptome Analysis

B16-bering mice were treated with 0.6×105 naïve Foxp3GFP Trp-1+ cells, irradiated GVAX and anti-CTLA-4 (Th condition) or irradiation (5Gy) and anti-CTLA-4 (Th-ctx condition). Control mice received naïve Trp-1 cells only (control). 8 days after transfer Trp-1+GFP− cells were FACS purified. RNA was isolated using TRIzol (Invitrogen). The GeneChip® Mouse Genome 430 2.0 Array (Affymetrix) was used to analyse the transcriptome. Raw expression values were normalised using the robust multi-array average (RMA) procedure (Irizarry, 2003) implemented in the package affy. Differential gene expression analysis was carried out on all genes, or a selection of previously described transcription factors (Gerstberger et al., 2014) in the package limma (Smyth, 2004). Gene set enrichment analysis (GSEA) was conducted using the package fgsea with 1000 permutations (Sergushichev, 2016), with reactome and MSigDB C7 signature sets (Godec et al., 2016). Correction for multiple testing was carried out using the Benjamini-Hochberg method. All microarray analyses were done in the R statistical programming environment.

FACS

Directly conjugated antibodies employed for flow cytometry are listed below. Staining of FoxP3 and Ki67 and Granzyme B was performed using the FoxP3 Transcription Factor Staining Buffer Set (ThermoFisher). Cytokine staining was performed using Cytofix/Cytoperm buffer set (BD Biosciences). For quantification of absolute number of cells, a defined number of fluorescent beads (Cell Sorting Set-up Beads for UV Lasers, ThermoFisher) was added to each sample before acquisition and used as a counting reference.

In Vitro Assays

Purified CD4+ T cells (CD4 T cells beads, Miltenyi or CD4 Dyna Beads, Invitrogen) were cultured in RPMI complete medium (as above) together with DC and irradiated feeder cells (40 Gy) in 2:1:1 ratio for 72 or 96 h. Polyclonal CD4+ T cells were stimulated with anti-CD3 (2C11) and anti-CD28 (37.51) (BioXcell); OT-II cells with OVA peptide (Pepscan) and Trp-1 cells with Trp-1 peptide (Pepscan) at concentration indicated in figure legend. Cells were additionally supplemented with IL-2, IL-15 or IL-7 (Peprotech) at indicated in figure legend concentration. Mouse CD4 T cells were cultured with aCD25 (PC61) and aIL-2 (JES6-1A12, BioXcell).

Human PBMCs were isolated from healthy volunteers' blood.

FACS-purified human CD4⁺ T cells were cultured in RPMI complete medium with irradiated autologous feeders cells in 1:1 ratio for 96 h, stimulated with aCD3 (OKT3) and aCD28 (9.3; BioXcell). To some wells anti-CD25 antibody (Basiliximab; Novartis) was added 48 hours post aCD3/CD28 stimulation.

For Treg suppression assays FACS-purified naïve mouse or human CD4+ T cells were co-cultured with autologous Treg cells at indicated ratios.

Generation of Trp-1 TCR Transduced Cells

The transduction procedure was performed according to (Hotblack et al., 2018). Briefly, the TRP1 TCR was cloned into the retroviral vector pMP71 with a 2A sequence separating the Vα3.2 and VP14 chains, followed by an internal ribosome entry site (IRES) truncated CD19 sequence. The TCR was codon optimized and also contains an extra cysteine residue in the constant chains to enhance pairing of the α and β chains. To generate TRP-1 retroviral particles, Phoenix-Eco (PhEco)-adherent packaging cells (Nolan Laboratory) were transiently transfected with retroviral vectors for the generation of supernatant containing the recombinant retrovirus required for infection of target cells, as described previously. The PhEco-adherent packaging cells were transfected using Genejuice (Merck) with the pCL-eco construct and the TRP-1 TCR vector according to the manufacturers' instructions. WT or Blimp-1 CKO CD4⁺ T cells were purified by magnetic selection according to the manufacturer's instructions (Miltenyi). Sorted cells were activated with concanavalin (Con) A (2 μg/mL) and IL-7 (1 ng/mL) for 24 hr, and then 2×106 activated T cells were incubated for a further 72 hr with retroviral particles on retronectin-coated (Takara-Bio) 24-well plates, in the presence of IL-2 (100 U/mL; Roche). Transduced cells were injected intravenously into mice 72 hr after transduction.

ELISPOT

Purified CD4 TILs and LN cells form MCA205 aCTLA-4 treated tumours were cultured on anti-GzmB coated ELISPOT Plate (R&D) for 24 h with unpulsed DC or MCA205-pulsed DCs and 50 □g/ml aMHC-II (M5/114) ELISPOT Assay performed according to manufacturer's protocol.

Quantitative qPCR

RNA from FACS-purified CD4+CD25lo TILs from MCA205 tumour was extracted with RNeasy micro kit (Qiagen) according to manufacturer's protocol. Amount of RNA was quantified with Qubit (ThermoFisher).

Synthesis of cDNA was carried out with SuperScript III reverse transcriptase (ThermoFisher). Purified cDNA was then used as template for the quantitation of the indicated genes using gene-specific primers (Table 3). qPCR was performed with QuantiTect Sybr Green PCR kit Syber reagents (Qiagen). Values were normalized and plotted according to the expression of Hprt1 in the same samples, using a ΔC_{T} method.

Data Analysis

Flow cytometry data were analyzed with FlowJo v10.0.8 (Tree Star). PhenoGraph analysis was performed using Cytofpipe v1.2 package (L. Conde, BLIC-UCL). Statistical analyses were done with Prism 7 (GraphPad Software); p values were calculated using one or two-way ANOVA with Tukey post-tests (ns=p>0.05,*p<0.05, **p<0.01, ***p<0.001, ****p<0.0001). Kaplan-Meier curves were analyzed with the log-rank test.

### Example 1—CD4⁺ TCR Transgenic T Cells Acquire a Polyfunctional Th/Cytotoxic Phenotype Upon Transfer into Tumour-Bearing Lymphopenic Hosts

Previous studies have demonstrated that melanoma-reactive tyrpl-specific TCR transgenic CD4⁺ T cells upregulate GzmB in addition to IFNγ and TNFα following transfer into lymphopenic hosts, acquiring potent cytotoxic activity (Quezada et al., 2010; Xie et al., 2010). To confirm whether this activity was specific to the Trp-1 TCR or driven by therapeutic modality, we analysed the activity of Trp-1-specific CD4⁺ cells (hereafter referred to as Trp-1 cells) in the context of host lymphodepletion combined with aCTLA-4 treatment or in response to a GM-CSF-expressing tumour cell based vaccine (GVAX) combined with aCTLA-4, which also induces effective Trp-1 cell activation and IFNγ secretion in vivo (Simpson et al., 2013). Briefly, B16 tumour-bearing mice were either left untreated or treated at day 8 either with total body irradiation (RT)+Trp-1+aCTLA-4, Trp1+GVAX+aCTLA-4, or Trp-1 cells in the absence of irradiation or vaccine as an additional control (referred to as control treatment, Trp-1 ctrl.) (Figure SlA). Transfer of Trp-1 cells into irradiated hosts in combination with aCTLA-4 promoted rejection of large, established tumours in all treated mice, whereas GVAX and aCTLA-4 failed to drive complete responses (FIGS. 1A and S1B). To investigate the cause of these different outcomes, we assessed the quantity and quality of Trp-1 cell infiltrates following therapy. Although both GVAX- and radiation-based therapies significantly enhanced Trp-1 effector cell (CD4⁺Trp-1⁺ Foxp3⁻) proliferation in the tumour microenvironment, irradiation lead to the largest, most significant increases in Trp-1 effector accumulation and ΔTeff/Treg ratio compared to Trp-1 monotherapy (Figure SlB). Both treatments (RT+Trp1⁺aCTLA-4 and GVAX+Trp-1+aCTLA-4) induced high levels of T-bet expression and IFNγ production by tumour infiltrating Trp-1 cells (FIG. 1B), suggesting acquisition of a Th1-like differentiation programme. In contrast, only Trp-1 CD4⁺ T cells primed in the lymphopenic environment (RT+Trp1+aCTLA-4) significantly upregulated GzmB expression, revealing a polyfunctional Th/cytotoxic phenotype (from this point referred to as Trp-1 Th-ctx) (FIG. 1C). TNFα and IL-2 followed a similar pattern, with the highest levels observed in Trp-1 Th-ctx cells (Figure SiC and data not shown). GVAX-expanded Trp-1 cells showed only T helper activity, with no significant upregulation of GzmB (from this point referred to as Trp-1 Th).

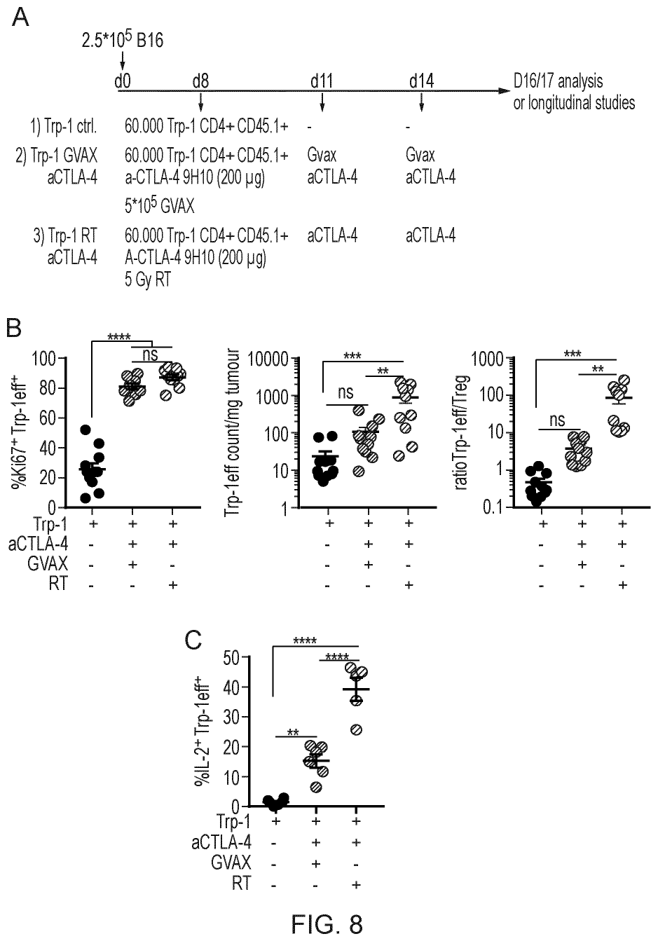

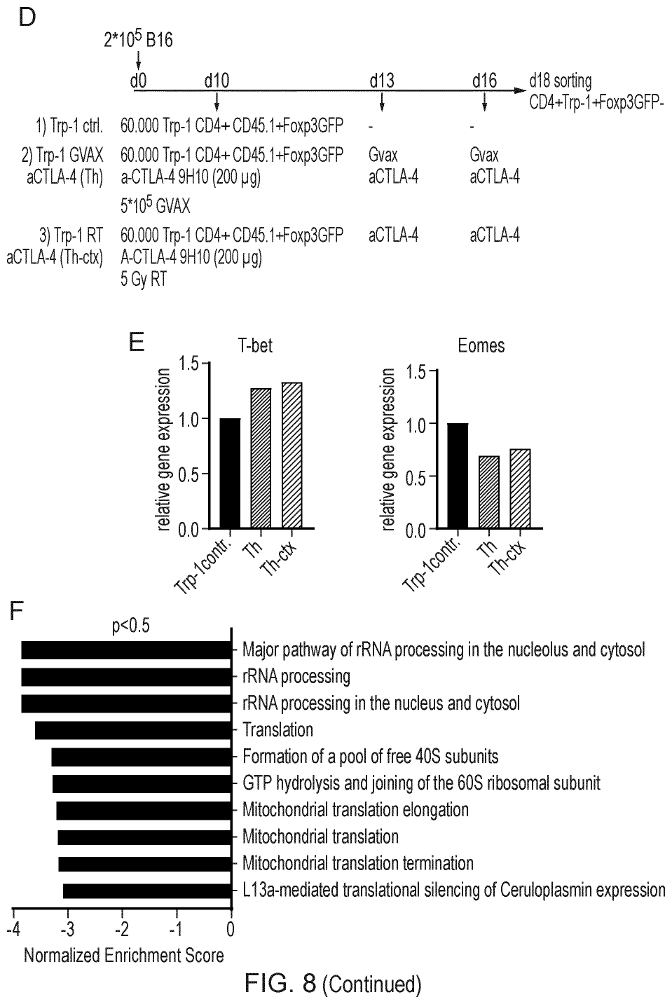

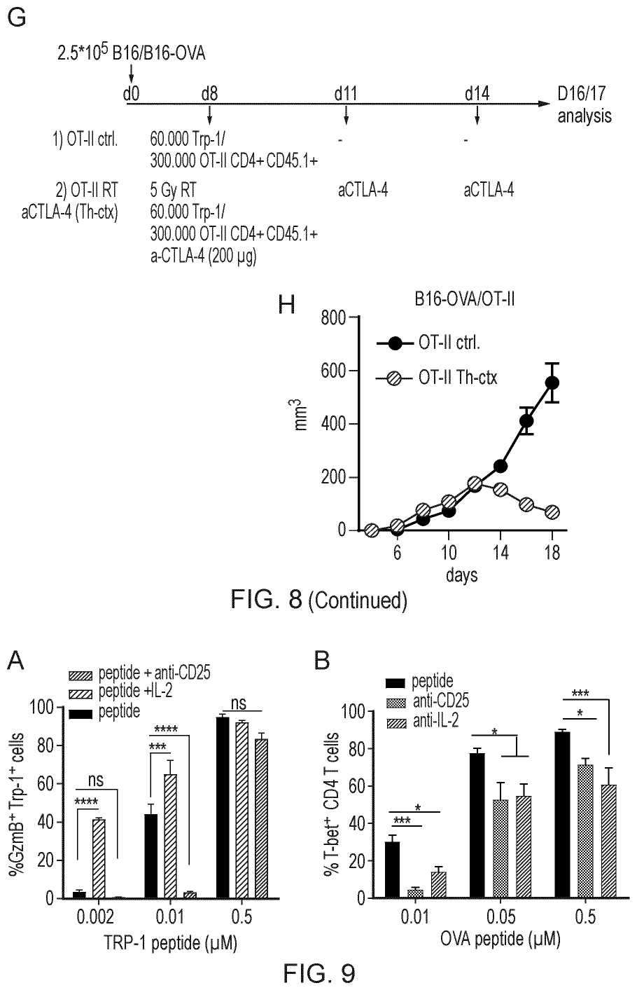

To gain insight into the molecular processes distinguishing Trp-1 Th-ctx cells from Trp-1 Th cells, we performed gene expression profiling on Trp-1 Foxp3⁻ cells isolated from tumour and draining lymph nodes (dLN) 8 days after treatment initiation. B16-bearing mice received Trp-1 cells as a monotherapy, or combined with GVAX+aCTLA-4 or with RT+aCTLA-4 treatment (FIGS. 1D and S1E). Comparison of Trp1 Th-ctx to Trp1 Th isolated from tumours identified 382 differentially expressed genes (p<0.01 and log 2 fold change ≥2) (Table 1). Gzmb, Gzma, Gzmk, Icam1, Tnf were found to be amongst the most highly upregulated genes in Trp-1 Th-ctx compared to Trp-1 Th cells in keeping with our prior phenotypic analyses. We also observed higher expression of genes previously reported to be upregulated in cytotoxic CD4⁺ T cells recognising viral antigens (Donnarumma et al., 2016), such as Ccl5, Ct1a2b and Cd7. Transcription factors upregulated in Trp1 Th-ctx cells (Table 2) included those belonging to the Kruppel-like factors family (KLf2, 7, 10), of which Klf2 has been shown to promote T-bet and Blimp-1 expression (Lee et al., 2015), as well as transcription factors with established roles in shaping CD4⁺ T cell fate, including Maf, Irf4, Prdml (Blimp-1) and Egr1 (Fu et al., 2017; Zhu and Paul, 2010) (FIG. 1E). When analysing the genes upregulated in GVAX-expanded Trp-1 Th compared to Trp1 Th-ctx we identified a set of genes previously reported to be associated with Tfh cells or natural Th21 cells, including Sostdcl, Stfa3, Tox, Ccr6, Tcf7 (encoding TCF-1), Gpm6b and Cd200 (Marnik et al., 2017; Choi et al., 2015) (FIG. 1E). Although the genes upregulated in Th-ctx cells do not specifically match a single defined CD4⁺ helper lineage (i.e. Th1, Th2, Th17, Th21), we noted that many differentially expressed genes were previously reported to be regulated by Blimp-1, including Socs1, Slamf1, Grap2, Maf, Ctla4 and Il-10 (Bankoti et al., 2017). T-bet was not differentially expressed between Th and Th-ctx cells, as its expression was upregulated in both conditions in comparison to control Trp-1 cells, in agreement with flow cytometry analyses (FIG. 8F). A Reactome pathway analysis revealed a significant upregulation of the apoptosis/survival related genes pathway, TLR-receptor activation, and cytokine signalling including CGAMMA chain receptor signalling pathways in Trp-1 Th-ctx cells in comparison to Trp-1 Th TILs (FIG. 1F). Furthermore, in keeping with the upregulation of CGAMMA chain cytokine signalling genes, a gene set enrichment (GSEA) analysis showed enrichment of IL-2 responsive genes (Castro et al., 2012) in Th-ctx conditions (FIG. 1F). There were no significantly enriched pathways that were directly related to the immune system in Trp-1 Th cells in comparison to Th-ctx cells (FIG. 8E).

To determine whether the acquisition of the polyfunctional Th1 and cytotoxic phenotype was specific to the Trp-1 system, we repeated these experiments using B16-OVA tumour and OVA-reactive CD4 OT-II TCR Tg T cells (FIG. 8F). Transfer of OT-II cells into irradiated, B16-OVA-bearing mice resulted in expansion of the OT-II population, GzmB upregulation, and rejection of established OVA-expressing melanoma (FIGS. 1G and S1G). Further characterisation of OT-II cells revealed, similar to Trp-1 cells, that GzmB-expressing OT-II cells also co-express T-bet (FIG. 1H). Thus, the observed Th-ctx phenotype is independent of TCR specificity. Furthermore, and as previously demonstrated for Trp-1 Th-ctx cells (Quezada et al., 2010), OT-II cells were able to directly kill B16-OVA tumour in vitro in a GzmB-dependent manner (FIG. 1I).

Taken together these data suggest that both therapeutic modalities (Gvax+aCTLA4 and RT+aCTLA4) promote Trp-1 T cells differentiation into a core polyfunctional T helper phenotype with marked Th1-like characteristics. However, whilst Gvax+aCTLA4 favoured a Th follicular-like signature in tumour infiltrating Trp1 cells, RT+aCTLA-4 supported the acquisition of additional transcriptional programmes associated with cytotoxicity (Th-ctx).

### Example 2—Endogenous IL-2 Drives Acquisition of GzmB Expression by Both Murine and Human CD4⁺ T Cells In Vitro

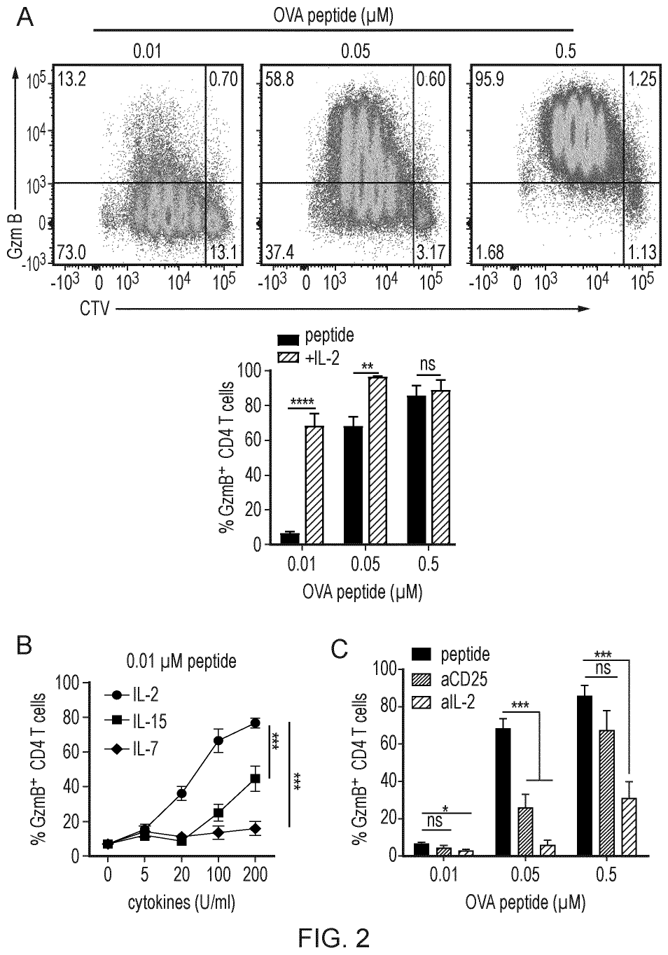

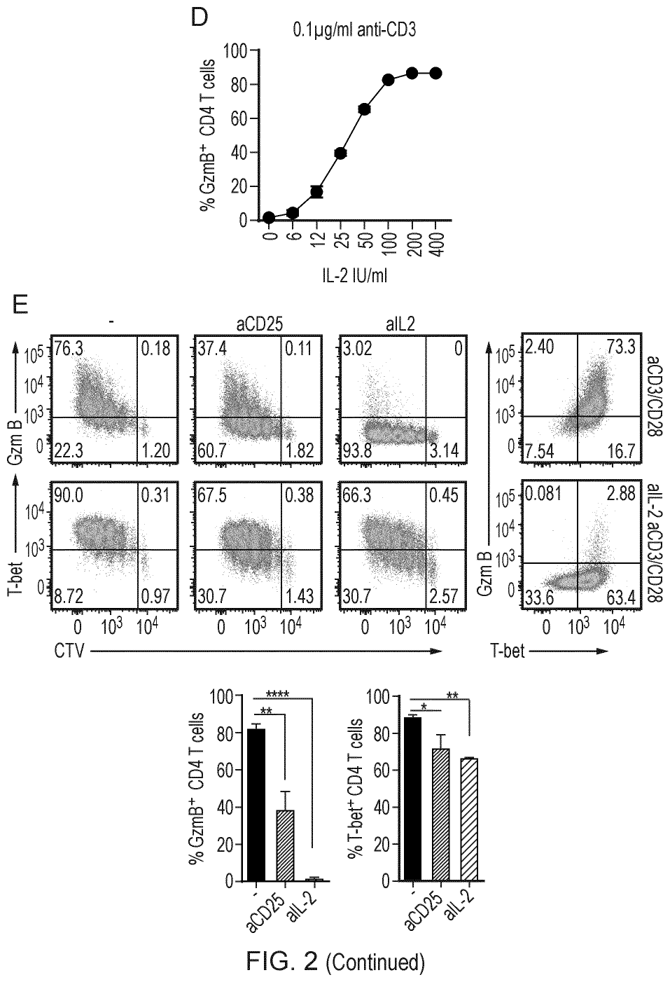

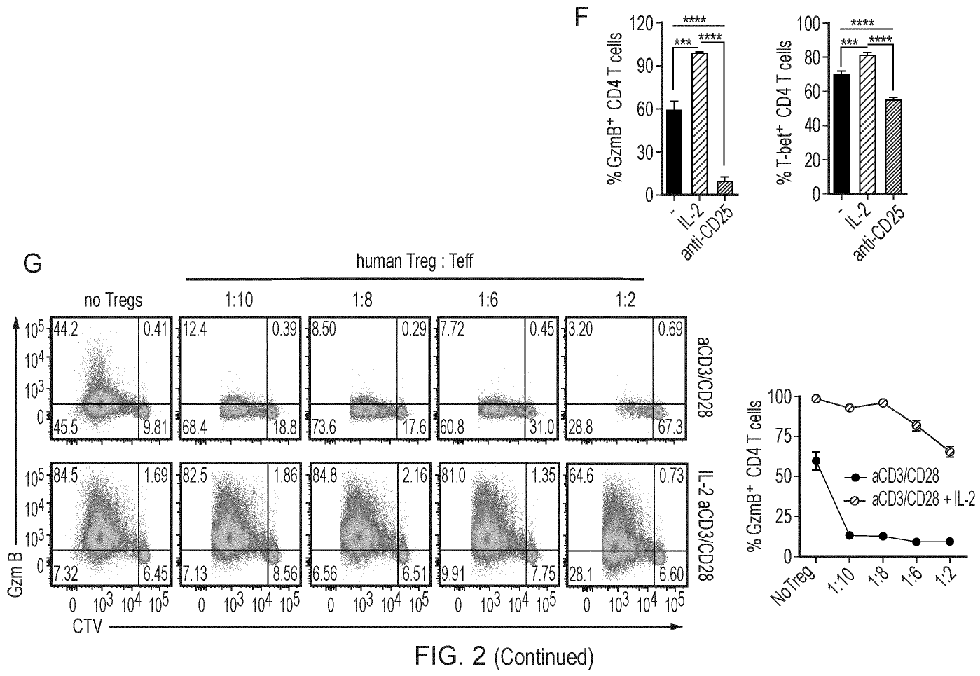

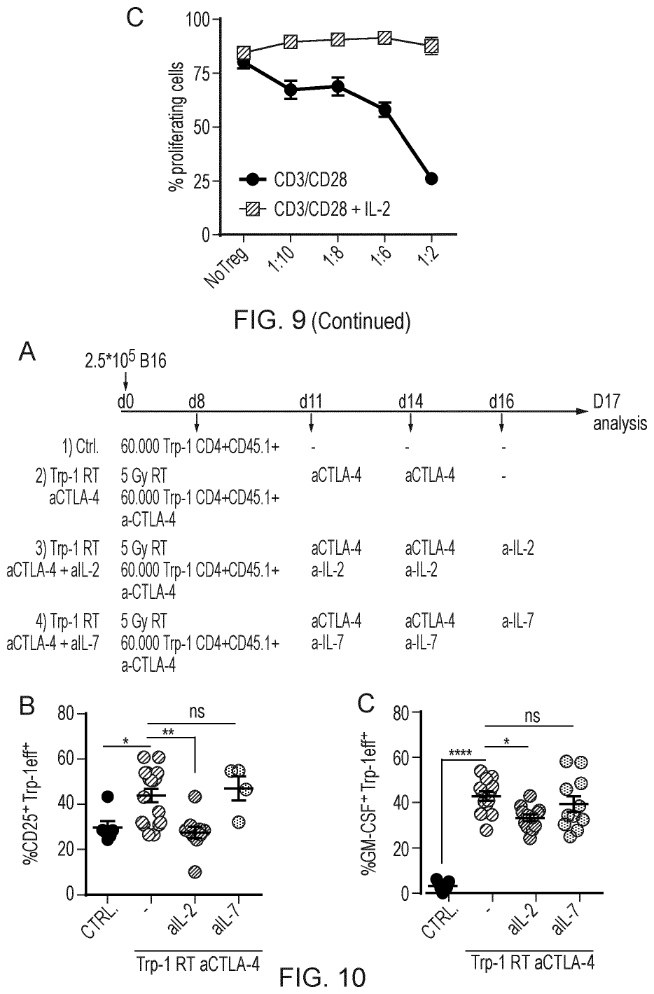

The increased expression of genes associated with CGAMMA chain cytokine signalling and the response to IL-2 in Trp1 Th-ctx is consistent with the cytokine milieu known to be induced by lymphodepletion (Williams et al., 2007) and could offer mechanistic insights into the acquisition of cytotoxic activity by tumour reactive CD4⁺ T cells in vivo. We therefore evaluated the potential contribution of CGAMMA receptor cytokines to the upregulation of GzmB in tumour-reactive CD4⁺ T cells. Briefly, CD4⁺Trp1 and OTII TCR Tg T cells were stimulated in vitro for three days with different amounts of cognate antigen and in the presence or absence of IL-2, IL-7 or IL-15. Both CD4⁺Trp1 and OT-II T cells upregulated GzmB in response to increasing antigen dose, whilst exogenous IL-2 further increased GzmB expression at lower antigen concentrations (FIGS. 2A and S2A). Of the CGAMMA chain cytokines evaluated, IL-2 was the most potent inducer of GzmB at low antigen concentration, followed by IL-15 and at a much lower level by IL-7 (FIG. 2B). Of key relevance, endogenous IL-2 was critical for GzmB upregulation in vitro as both IL-2 neutralisation and CD25 receptor blockade reduced GzmB upregulation in response to antigen (FIGS. 2C and 9A).

The high levels of T-bet expression in CD4⁺Trp1 and OTII Th-ctx cells lead us to evaluate its possible association with IL-2 and GzmB upregulation. Whilst IL-2 deprivation reduced T-bet expression by in vitro activated OT-II cells (FIG. 9B), the impact on T-bet appeared less marked than the impact on GzmB, suggesting that these two pathways may not be directly linked. Further validation was obtained using polyclonal mouse CD4⁺ T cells stimulated in vitro with αCD3 in the presence or absence of exogenous IL-2, aCD28 and/or neutralising anti IL-2. In keeping with the CD4⁺ TCR Tg data, increasing amounts of exogenous IL-2 significantly augmented GzmB expression in polyclonal CD4⁺ T cells stimulated with low doses of αCD3 (FIG. 2D), and endogenous IL-2 was critical for GzmB upregulation in polyclonal CD4⁺ T cells stimulated with αCD3/αCD28 (FIG. 2E). Whilst IL-2 neutralization had a potent negative impact on GzmB expression, a less striking change in T-bet expression was observed. Thus, IL-2 neutralisation resulted in an almost complete ablation of GzmB expression with minimal impact on T-bet expression (FIG. 2E).

We next tested whether endogenous IL-2 was required for the differentiation of human Th-ctx cells. Sixty percent of aCD3/aCD28-stimulated naïve CD4⁺ T upregulated GzmB, with the addition of exogenous IL-2 significantly increasing the percentage of GzmB⁺CD4s (ninety five percent). In contrast blockade of IL-2R signalling with an anti-human CD25 (Basiliximab) significantly reduced GzmB expression to levels below those of the untreated control sample (FIG. 2F).

IL-2 deprivation is thought to be an important mechanism utilised by Treg cells to suppress T cell-mediated immunity, primarily impacting on proliferation and survival (Sakaguchi et al., 2008). To determine whether Tregs also suppress acquisition of cytotoxic potential by CD4 cells, we activated purified human naïve CD4⁺ T cells with αCD3/αCD28 and co-cultured them with different numbers of autologous Treg cells. Very low numbers of Treg cells (1:10 Treg:Teff) significantly suppressed the expression of GzmB, whereas T-bet expression was only partially affected (FIG. 2G and data not shown). Interestingly, a much higher ratio of Treg:Teff (1:6) was needed in order to effectively suppress CD4⁺ Teff proliferation in vitro (FIGS. 2G and 9C). Finally, exogenous IL-2 was able to revert both effects, increasing GzmB production and proliferation by CD4⁺ Teff cells even at the highest Treg:Teff ratios. Taken together, these data suggest that endogenous IL-2 drives GzmB upregulation on CD4+Teff cells whilst regulatory T cells negatively control this process potentially through IL-2 competition.

### Example 3—Endogenous IL-2 Contributes to Upregulation of GzmB by Adoptively Transferred Tumour Reactive T Cells In Vivo

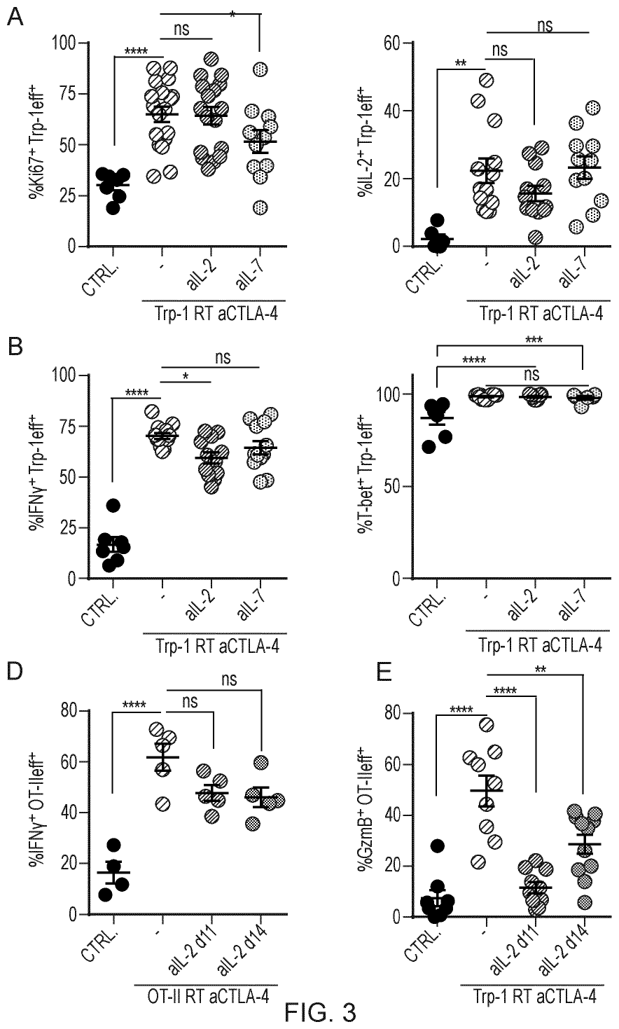

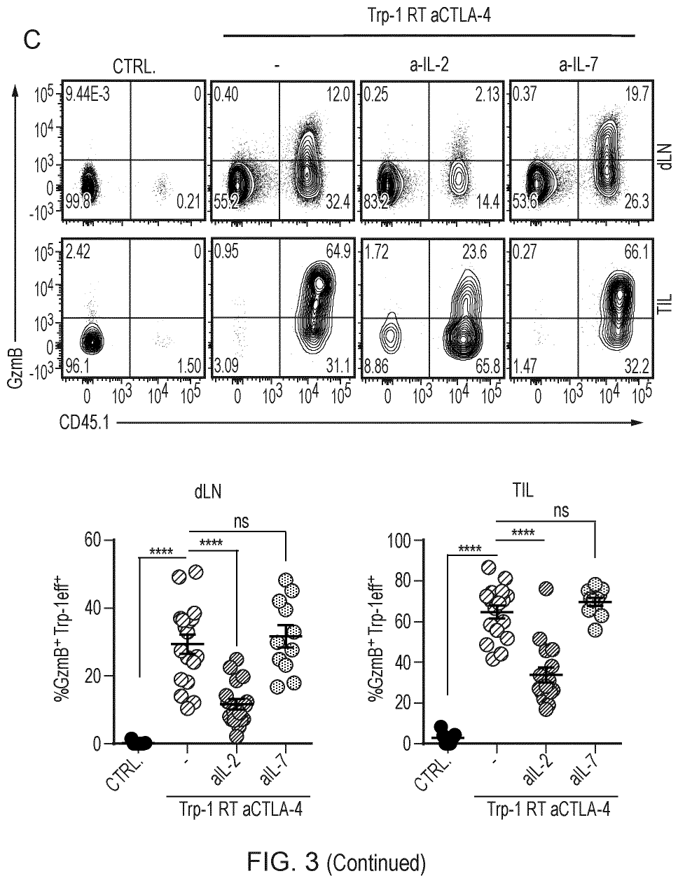

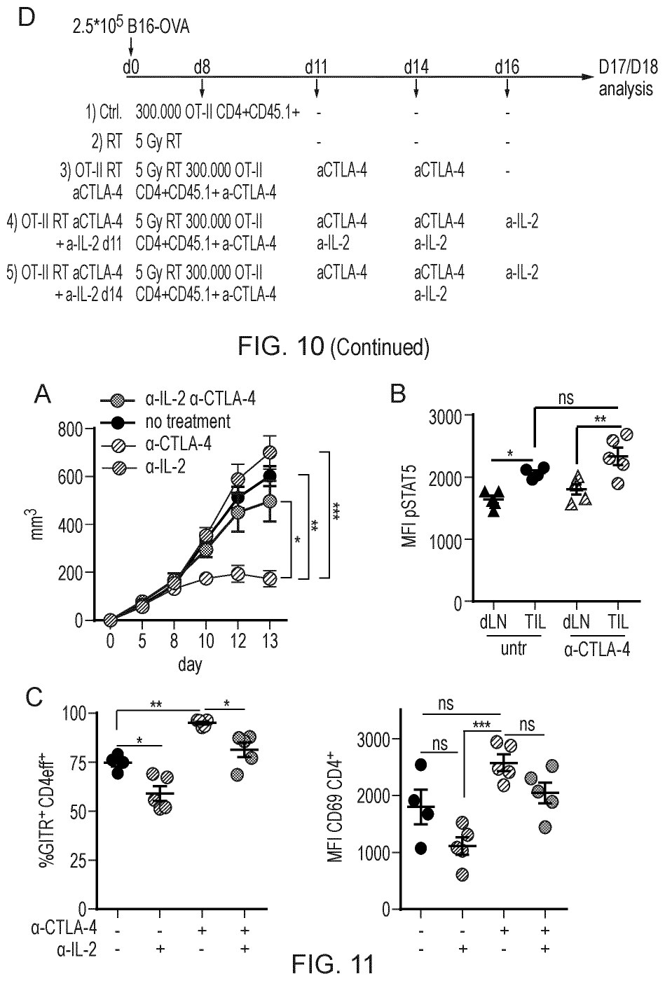

Next we sought to determine whether IL-2 controls GzmB expression in adoptively-transferred tumour-reactive CD4⁺ T cells in vivo. Briefly, eight days post-B16. B16 tumour implantation, mice received Trp1 cells alone (ctrl) or combined with irradiation and aCTLA-4 treatment (Trp-1 RT aCTLA-4) in the presence or absence of an aIL-2 neutralising antibody. An additional group of mice received an aIL-7 neutralising antibody to rule out its potential role in GzmB regulation in vivo, as this has been previously shown to be relevant for CD8⁺ T cells (Li et al., 2011). IL-2 or IL-7 neutralisation started 3 days after adoptive transfer to allow the initial expansion of transferred T cells (FIG. 10A). Trp-1 cells expanded in the tumour microenvironment in all three treatment conditions. IL-2 neutralization did not decrease the fraction of Ki67-expressing cells within the Trp-1⁺ compartment in contrast to aIL-7 treatment which significantly reduced CD4⁺Trp1 cell proliferation (FIG. 3A). Tumour infiltrating Trp-1 Th-ctx cells (Trp-1 RT aCTLA-4 condition) expressed high levels of IL-2 with neither anti-IL-2 nor anti-IL-7 treatment impacting its expression (FIG. 3A). As expected, IL-2 neutralisation resulted in decreased expression of the high affinity IL-2 receptor CD25 on activated CD4⁺ T cells (FIG. 10B). We further analysed how cytokine neutralization in the adoptive transfer settings impacted CD4⁺ T cell effector function. We observed a small decrease in IFNγ expression upon IL-2 neutralization, although T-bet expression was not significantly reduced (FIG. 3B). Among the cytokines we analysed in these settings, GM-CSF expression was downregulated in the tumour but not in draining LN by IL-2 neutralization (FIG. 10C and data not shown). The frequency of GzmB-expressing cells within the Trp-1 compartment was, however, significantly downregulated in aIL-2 treated mice both in the draining LN and tumour (FIG. 3C). aIL-7 did not have an impact on GzmB expression by transgenic CD4⁺ T cells recapitulating the in vitro data (FIG. 3C). We performed similar studies using B16-OVA tumour and OT-II cells to evaluate if IL-2 has a similar impact on Th-ctx cells with different TCR specificity (FIG. 10C). In these experiments we started aIL-2 treatment at two different time-points post OT-II T cell transfer based on previous experiments defining the temporal profile of GzmB expression (data not shown). At a later time point (day 5 post transfer) the GzmB-expressing CD4⁺ OT-II T cells were already present in the tumour microenvironment. Similar to the B16/Trp-1 model, aIL-2 treatment caused a moderate decrease in IFNγ expression by CD4⁺ OT-II cells without changing T-bet expression (FIG. 3D and data not shown). The fraction of OT-II cell with lytic potential was significantly decreased when the aIL-2 treatment started 3 or 5 days post-transfer (FIG. 3E and data not shown). These findings support the hypothesis that IL-2 plays a central role in regulating the acquisition of a Th-ctx phenotype in the adoptive transfer setting.

### Example 4—Increased IL-2 Availability after Treg Depletion Contributes to Shaping T Helper Cell Phenotype in the Tumour Microenvironment

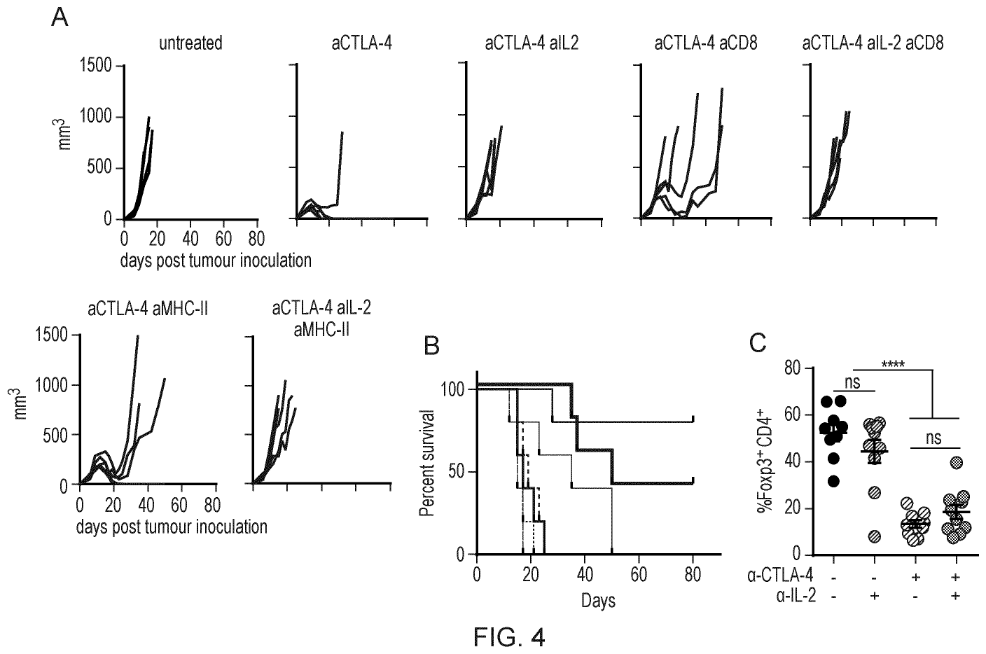

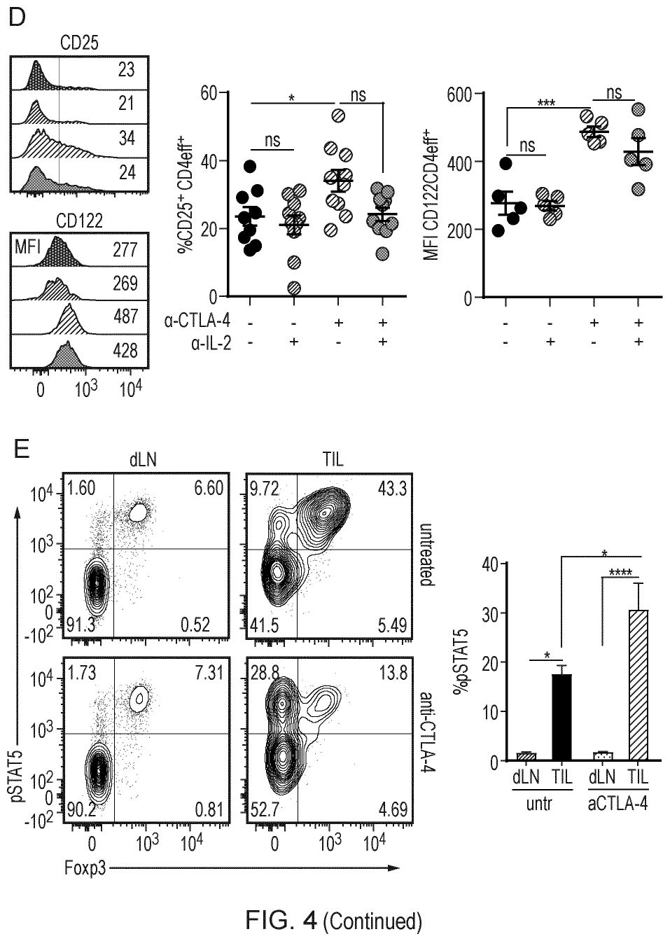

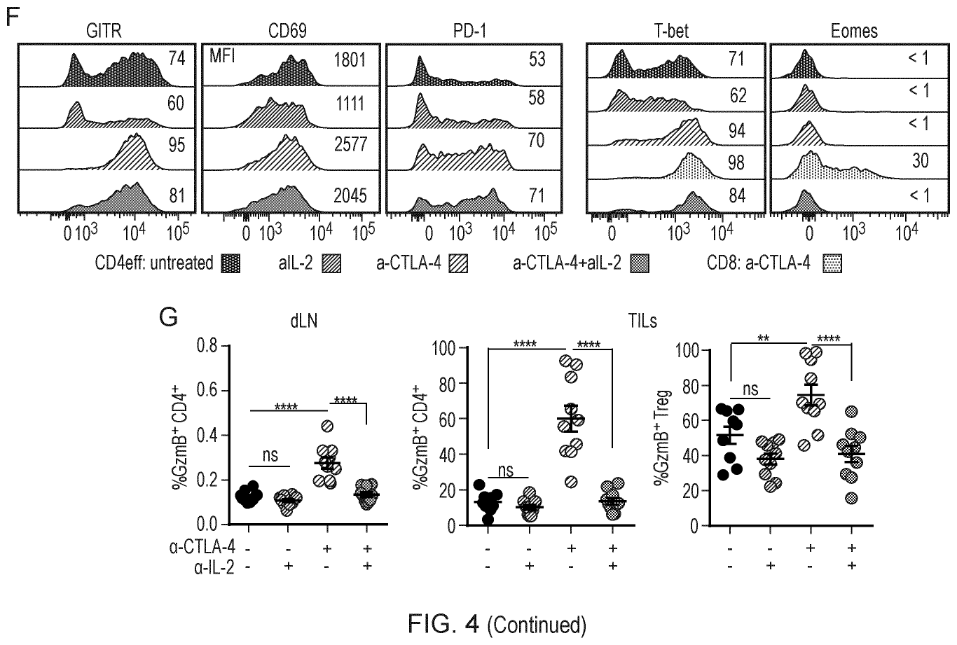

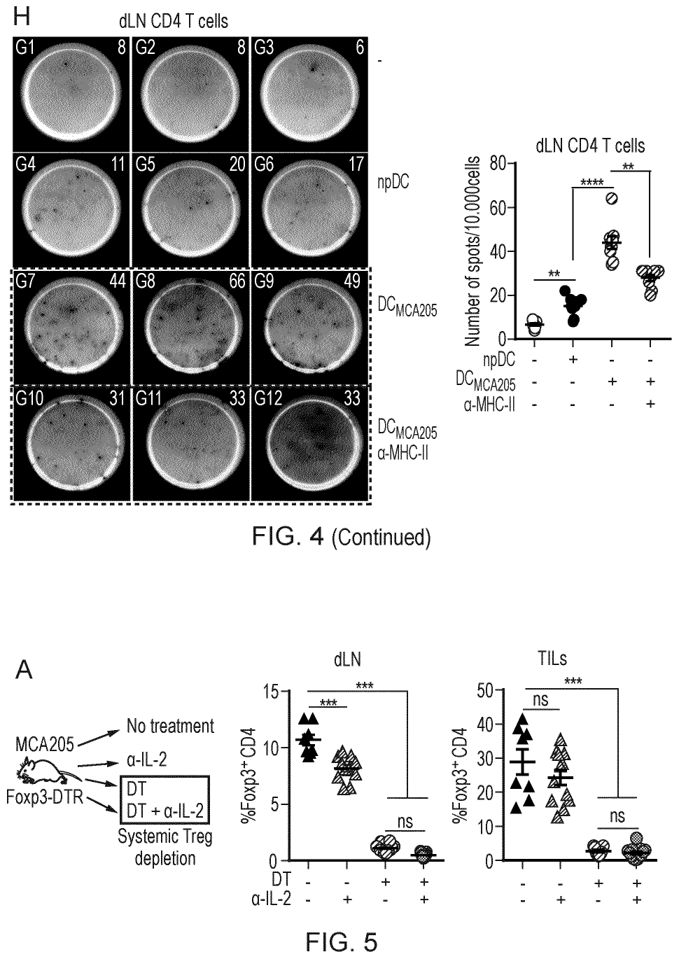

In ACT models lymphodepletion acts by enhancing the effector function of transferred T cells and increasing their ability to express IL-2 (Gattinoni et al., 2005), whilst aCTLA-4 is known to cause Treg depletion in mouse models of cancer via FcgR engagement (Arce Vargas et al., 2018; Selby et al., 2013; Simpson et al., 2013). Thus both, RT and aCTLA-4 increase the amounts of available IL-2 in vivo. To simplify our model and to determine whether these findings were relevant in the context of an endogenous (not TCR Tg) T cell compartment, we evaluated the role of IL-2 in the acquisition of cytotoxic activity by CD4⁺ T cells in a mouse model of cancer known to respond to aCTLA-4 monotherapy. To further evaluate if the increase in IL-2 concentration in the tumour was necessary for the anti-tumour activity observed following aCTLA-4, we inoculated WT mice with MCA205 and treated with anti-CTLA-4 antibody (clone 9H10) with or without neutralising anti-IL-2. In agreement with previous studies (Hannani et al., 2015), neutralisation of IL-2 abolished the anti-tumour activity of this Treg-depleting antibody. Furthermore, both CD4⁺ and CD8⁺ T cells were required for a-CTLA-4-mediated MCA205 tumour control (FIGS. 4A and 4B).

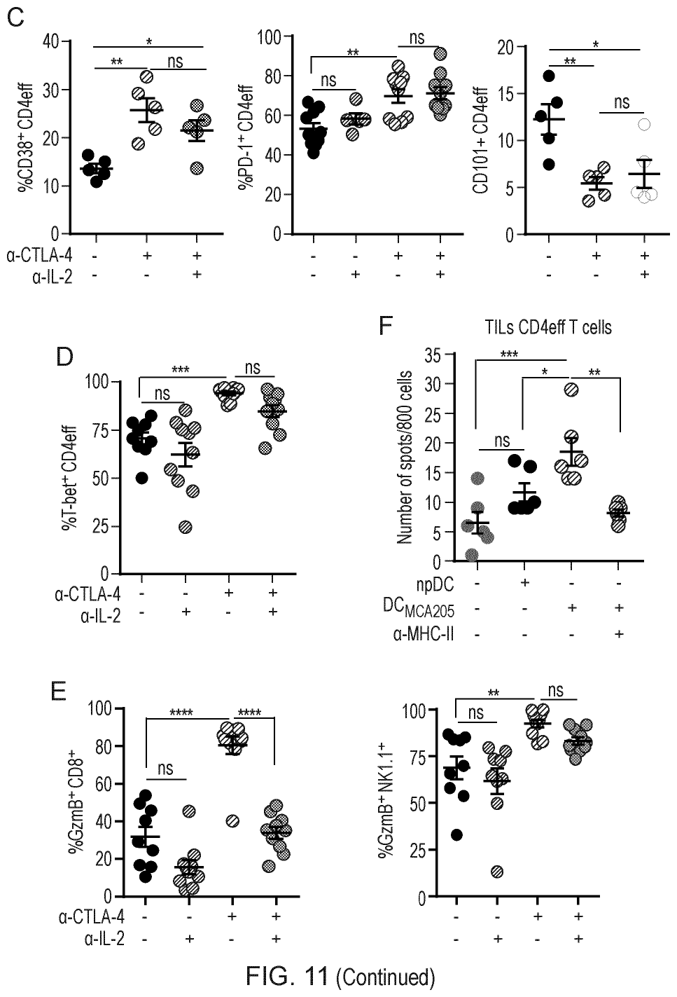

We sought to identify the aspects of aCTLA-4-mediated CD4⁺ T cell activation and differentiation that are dependent on IL-2 by analysing the quantitative and qualitative differences between TILs in aCTLA-4 versus combined treatment conditions. Briefly, tumour bearing mice were treated or not with aCTLA-4 and in presence or absence of neutralising aIL-2 antibodies. Tumour size was measured up to day 13 to ascertain the impact of the different treatment prior to assessment of CD4⁺ T cell differentiation within tumours (FIG. 11A). As IL-2 plays an important role in regulatory T cells homeostasis and function (Ye et al., 2018), we compared the frequencies of Tregs within the CD4⁺ TIL compartment across all conditions but found no significant differences between mice receiving aIL-2 and their respective controls (FIG. 4C). We further assessed the expression and activation of IL-2 signalling pathway components in CD4⁺ TILs and LN cells in the MCA205 tumour model. Both subunits of the IL-2 receptor were upregulated by CD4⁺ effector T cells in aCTLA-4 treated tumours. IL-2 neutralisation lead to decreased CD25 expression by CD4⁺ T cells in comparison to relevant controls in contrast to CD122 (IL-2R□), which was not affected by aIL-2 treatment (FIG. 4D). To determine whether increased expression of high-affinity IL-2 receptor by activated CD4eff (CD4⁺ Foxp3⁻) TILs translates into elevated IL-2 signalling, MCA205 TILs and LN cells were restimulated for 10 min with IL-2 and phosphorylation of STAT5 was measured. An increased level of pSTAT5 confirmed activation of the IL-2 pathway in CD4⁺ effector TILs in both untreated and aCTLA-4-treated tumours in comparison to LN CD4⁺ T cells (FIGS. 4E and 11B). Further analysis of T cell activation markers revealed increased expression of CD69, CD44, GITR and CD38 upon aCTLA-4 treatment, of which only GITR (Michael McNamara, 2014) was downregulated by IL-2 deprivation in comparison to respective control conditions (FIG. 4F, 11C and data not shown). PD-1 was consistently upregulated and the negative co-stimulatory molecule CD101 downregulated (Schey et al., 2016) in all aCTLA-4 treated groups regardless of IL-2 deprivation. Similarly to the adoptive cell transfer models, activated CD4⁺ T cells acquired a Th1 phenotype, expressing high levels of T-bet but, in contrast to CD8⁺ T cells, no Eomes (FIGS. 4F and 11C). The fraction of GzmB-expressing T cells was increased in the tumour following aCTLA-4 treatment, but not in draining LN. When the treatment was combined with aIL-2, however, the GzmB level in TILs was decreased to the level of untreated mice (FIG. 4G), in contrast to NK cells, which showed no significant change (FIG. 11E). Importantly, we found that Th-ctx CD4⁺ T cells isolated from aCTLA-4-treated MCA205 tumour are able to release GzmB when co-cultured with DC loaded with MCA205 tumour in an MHC-II-dependent manner (FIGS. 4H and 11F). Thus IL-2 is critical for GzmB upregulation by endogenous tumour-infiltrating CD4⁺ T cells with little impact on other features of CD4⁺ T cell activation and differentiation post aCTLA-4 therapy.

### Example 5—Treg Depletion Alone Drives Acquisition of GzmB Expression by CD4⁺ T Cells

To further explore the hypothesis that increased IL-2 availability is a crucial factor for the differentiation of CD4 Th-ctx after CTLA-4-driven Treg depletion, we used Foxp3-DTR mice to allow specific depletion of Tregs in absence of CTLA-4-blockade. We challenged Foxp3-DTR mice with MCA205 tumours and treated them with DT with or without aIL-2 (FIG. 12A). In contrast to aCTLA-4-mediated Treg depletion that is restricted to the tumour microenvironment, DT efficiently depletes Treg systemically (Kim et al., 2007) (FIG. 5A). This lead to a general activation of CD4⁺ and CD8⁺ T cells in both dLN and tumour. Combining DT-mediated Treg depletion with aIL-2 antibody treatment had little impact on Ki67 (FIGS. 5B and 12B), T-bet, IFNγ nor GM-CSF expression by CD4⁺ T cells in both LN and tumour (FIGS. 5C, 5D and 12C). In marked contrast, Treg depletion mediated GzmB upregulation in TILs and in dLN T cells, which was efficiently abrogated by aIL-2 antibody treatment in both CD4⁺ and CD8⁺ T cells (FIGS. 5E and 12D). We performed further experiments to assess GzmB expression at different time-points post-DT treatment, allowing correlation with Treg cell recovery. The expression of GzmB decreased exponentially with increasing frequency of Tregs within the LN CD4⁺ T cell compartment (FIG. 5F). Together, these data suggest that the increased availability of IL-2 caused by reduction in the Treg to CD4eff ratio is one of the key factors contributing to acquisition of a cytotoxic phenotype.

### Example 6—In Vivo Acquisition of Cytotoxic Phenotype by CD4⁺ TILs and Tumour Rejection are Independent of T-Bet Expression

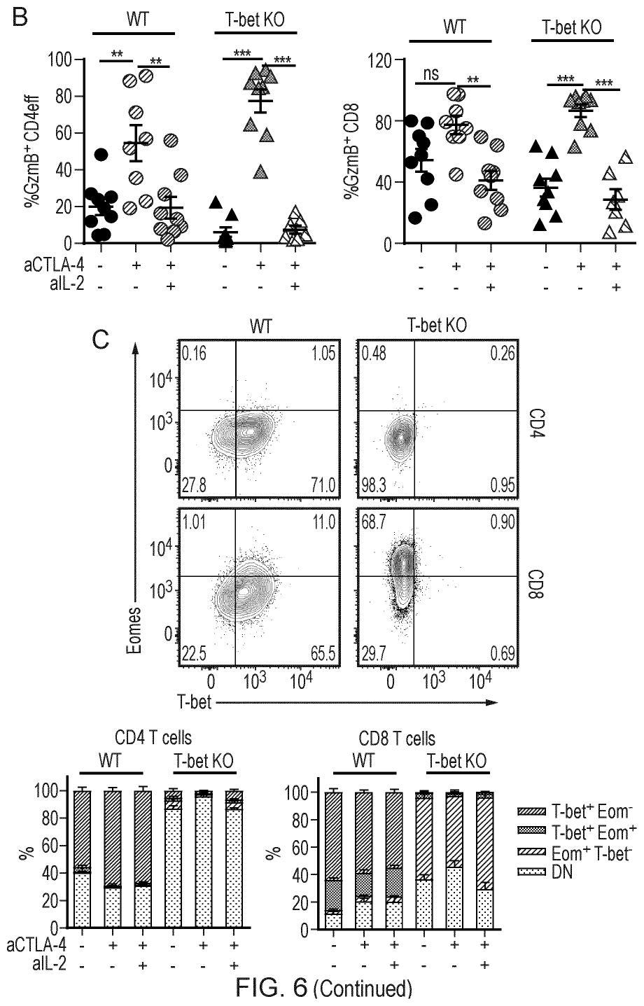

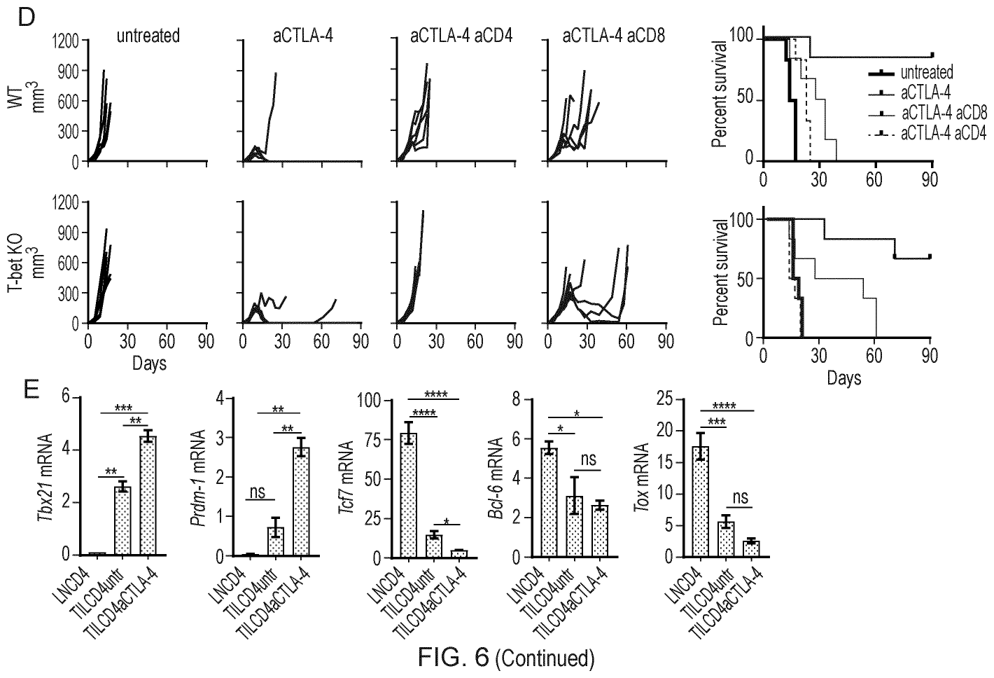

In all analysed conditions, in vitro and in vivo, CD4⁺ GzmB⁺ cells co-expressed the Th1 lineage-specifying transcription factor T-bet. To investigate if T-bet has a dual role, responsible for acquisition of both Th1 and cytotoxic features by CD4⁺ T cells in the tumour microenvironment, we inoculated T-bet^{-/-} and WT mice with MCA205 tumour and treated them with aCTLA-4 alone or in combination with neutralizing aIL-2. Anti-CTLA-4 mediated Treg depletion in both WT and T-bet^{-/-} mice but failed to promote IFNγ expression in T-bet-deficient T cells (FIG. 6A and data not shown). Of relevance, WT and T-bet-deficient CD4eff and CD8 TILs in aCTLA-4 treated tumours expressed equal levels of GzmB, and lack of T-bet had no effect on GzmB downregulation following IL-2 neutralization (FIG. 6B). These data indicate that T-bet is not required for the increase in GzmB expression mediated by IL-2 in CD4⁺ TILs. The transcription factor Eomes has been reported to compensate for T-bet deficiency in CD4⁺ T cells (Yang et al., 2008). To exclude this possibility, we compared expression of Eomes in WT and T-bet^{-/-} GzmB⁺ TILs. In marked contrast to T-bet-deficient GzmB⁺ CD8⁺ T cells, which express high level of Eomes, GzmB⁺ T-bet-deficient CD4⁺ T cells remained Eomes negative (FIG. 6C). These data suggest that Eomes is unlikely to drive GzmB expression in CD4⁺ TILs in the MCA205 model, even in the absence of T-bet. To further assess whether T cells unable to express IFNγ but maintaining cytotoxic activity are able to control MCA205 tumour growth, WT and T-bet^{-/-} mice were inoculated with fibrosarcoma tumours and treated with aCTLA-4 alone or in combination with T-cell depleting antibodies (FIG. 6D). We found that aCTLA-4 treatment increased the survival of T-bet-deficient mice and that T-bet-deficient CD4⁺ T cells were able to control tumour growth even more effectively than WT CD4⁺ T cells in CD8-depleted mice. Thus, T-bet is not required for aCTLA-4-mediated tumour rejection.

After excluding T-bet and Eomes as transcription factors driving GzmB expression in CD4⁺ T cells, we further analysed the expression of other candidate transcription factors in untreated and aCTLA-4 treated tumours. Based on our Trp-1 gene expression data and previous studies with viral antigen-specific CD4⁺ T cells (Donnarumma et al., 2016), we compared the level of Blimp-1 (Prdml), Tcf-1 (Tcf7), Tox (Tox) and Bcl-6 (Bcl6) mRNAs between non-activated CD4eff and CD4eff TILS in untreated and treated conditions. Consistent with our Trp-1 data, polyclonal Th-ctx cells infiltrating MCA205 tumours in aCTLA-4-treated animals upregulate Blimp-1 and downregulate Tcf7 and Tox in comparison to LN and TIL CD4⁺ T cells from untreated animals (FIG. 6E). Bcl6 expression was downregulated in TILs in comparison to LN CD4⁺ T cells but there was no difference between TIL from treated and untreated mice. These data suggest that the cytotoxic activity of CD4⁺ TILs in aCTLA-4 treated mice may be regulated independently of Th1 lineage specification.

### Example 7—IL-2 Controls Cytotoxic CD4⁺ T Cell Differentiation in a Blimp-1-Dependent Manner

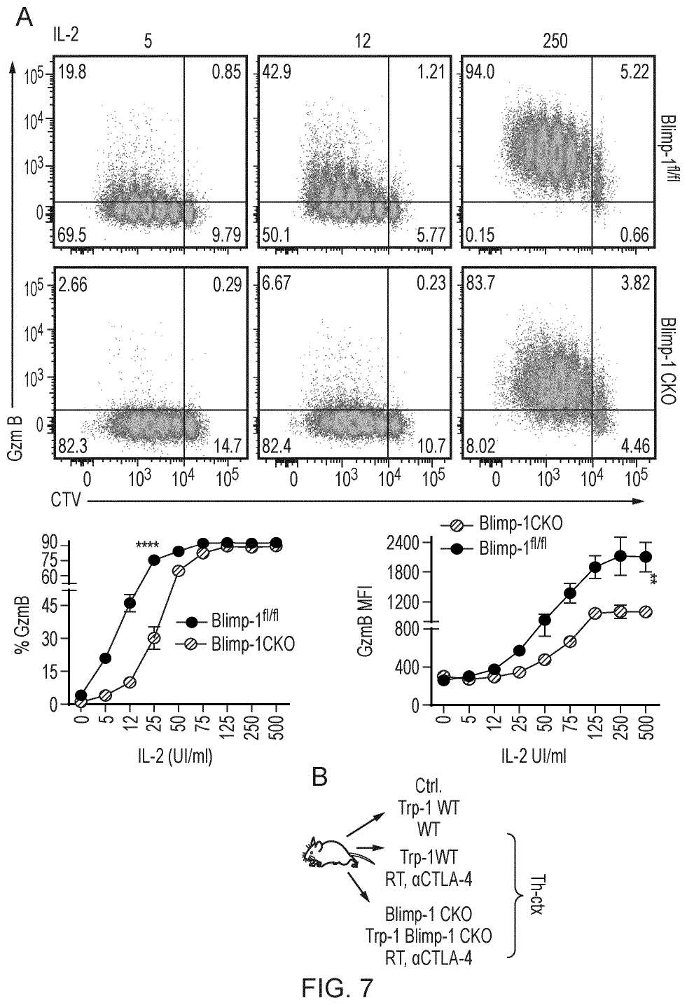

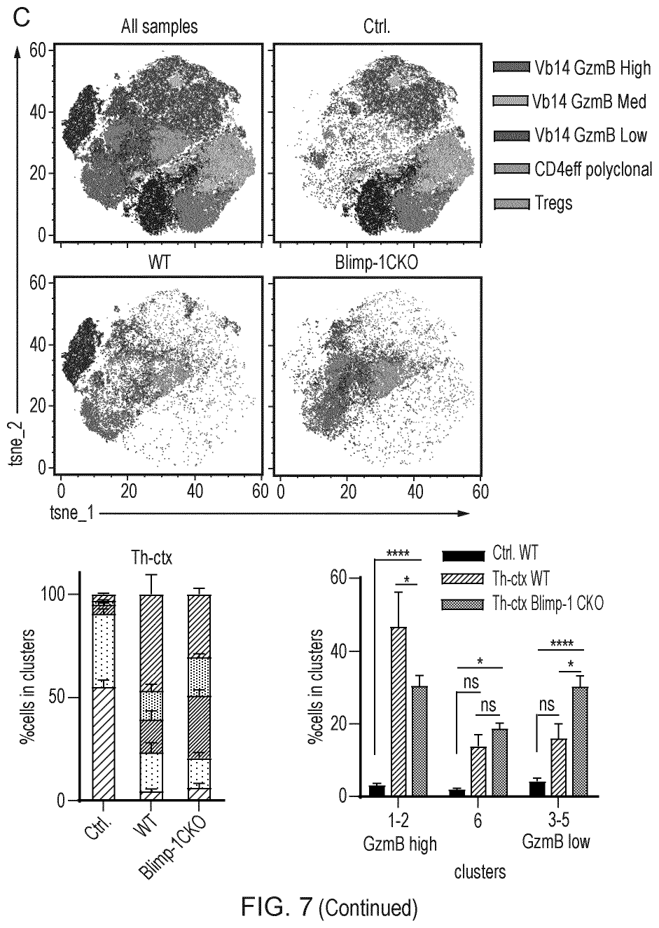

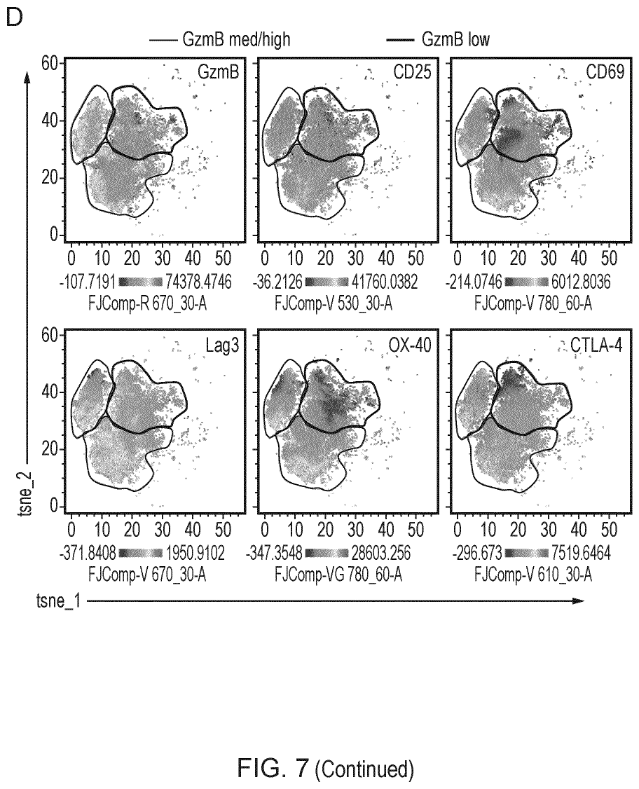

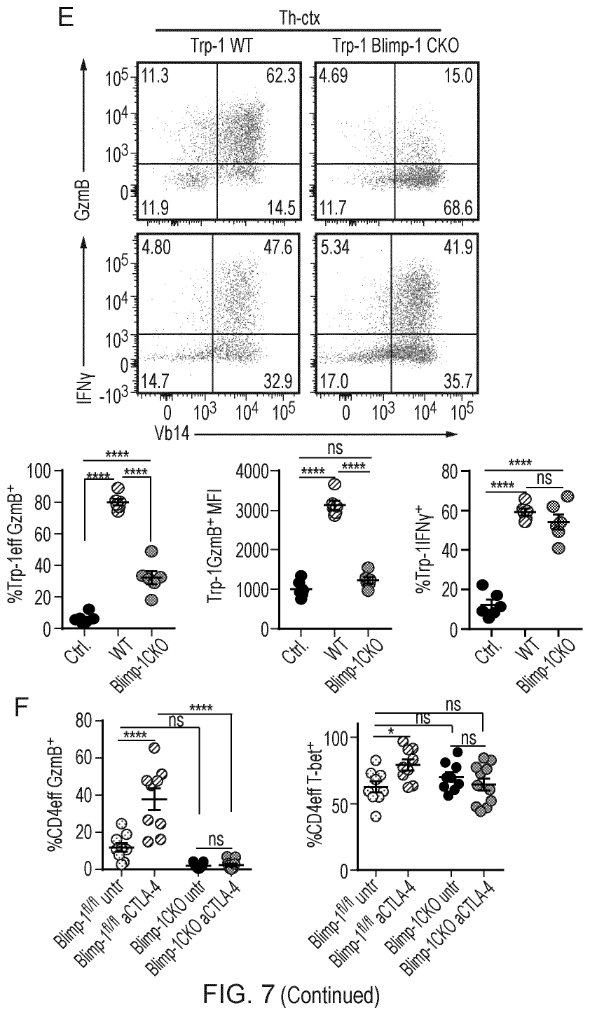

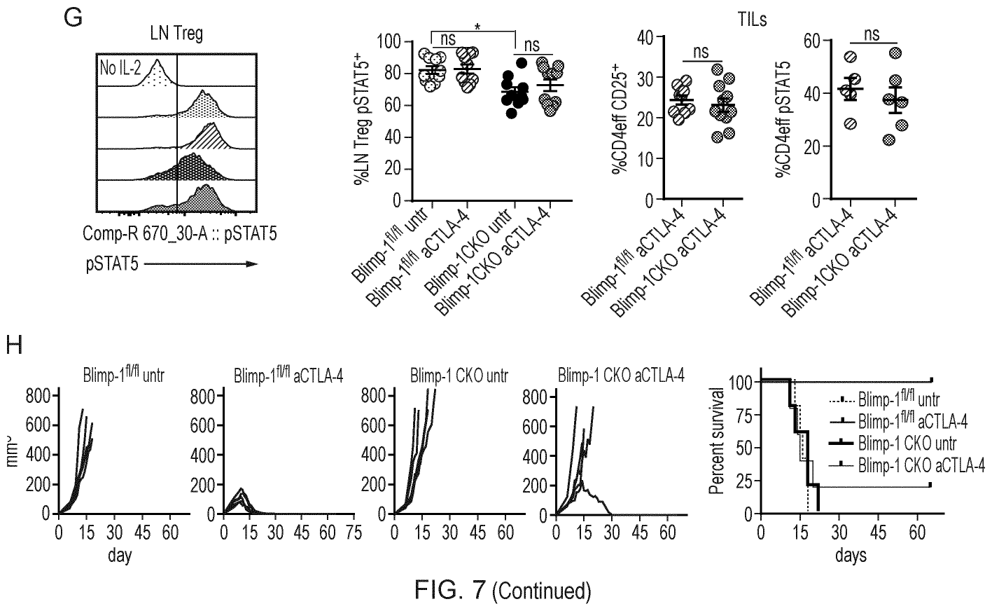

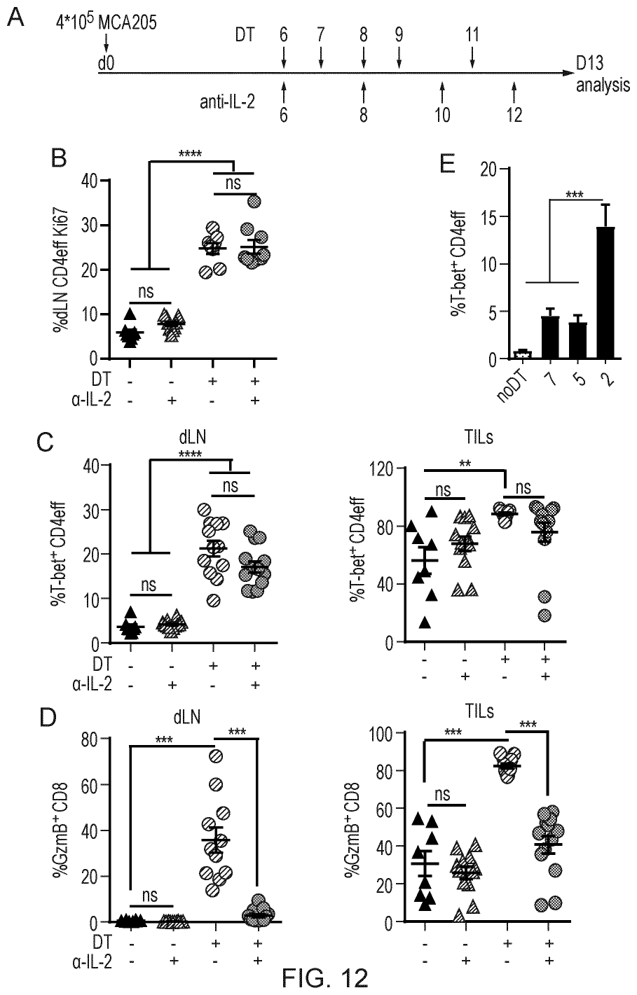

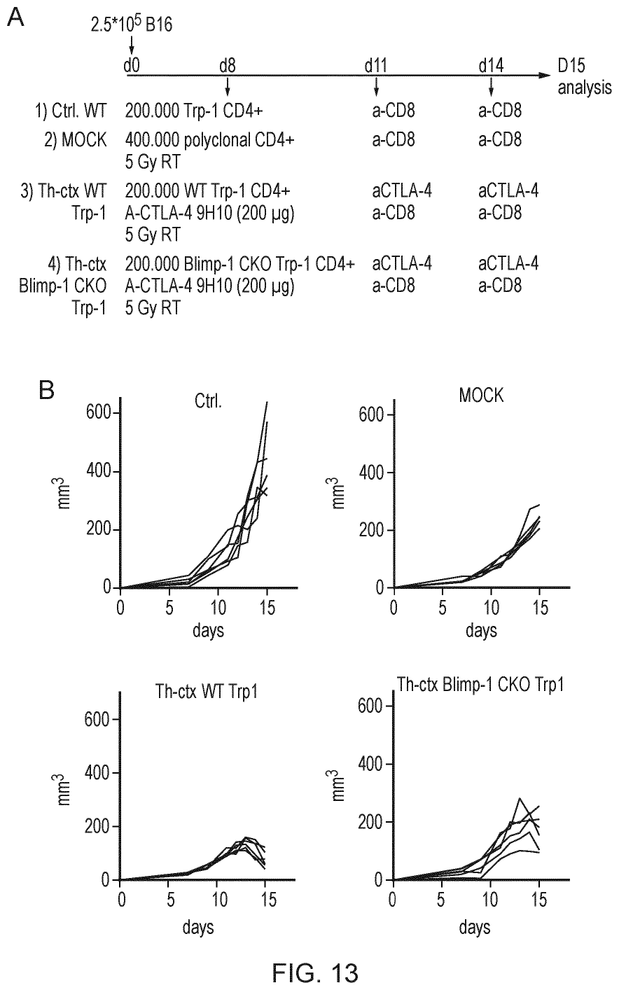

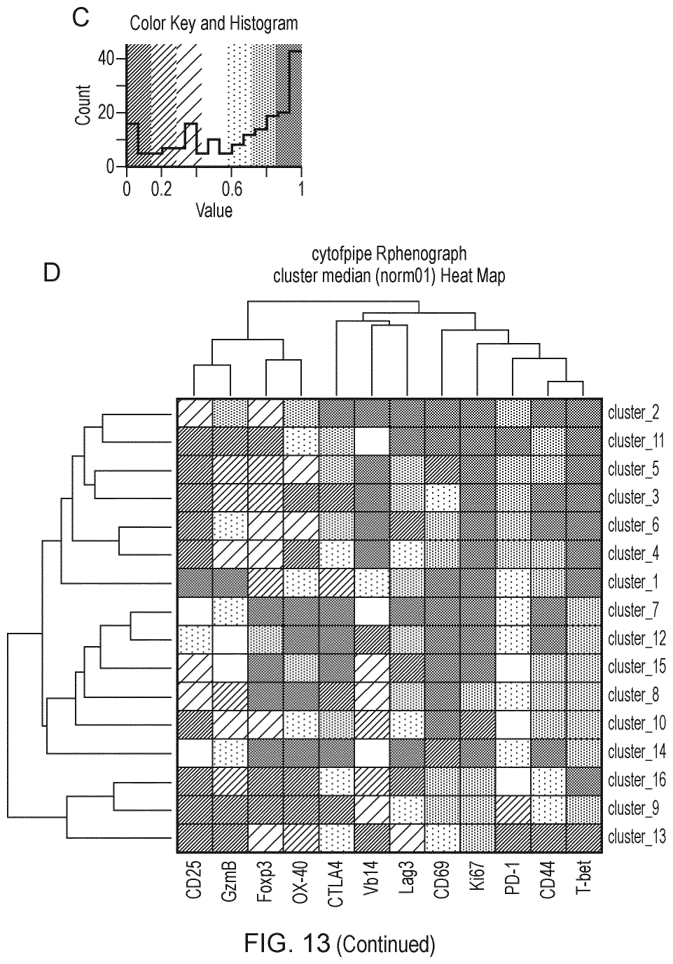

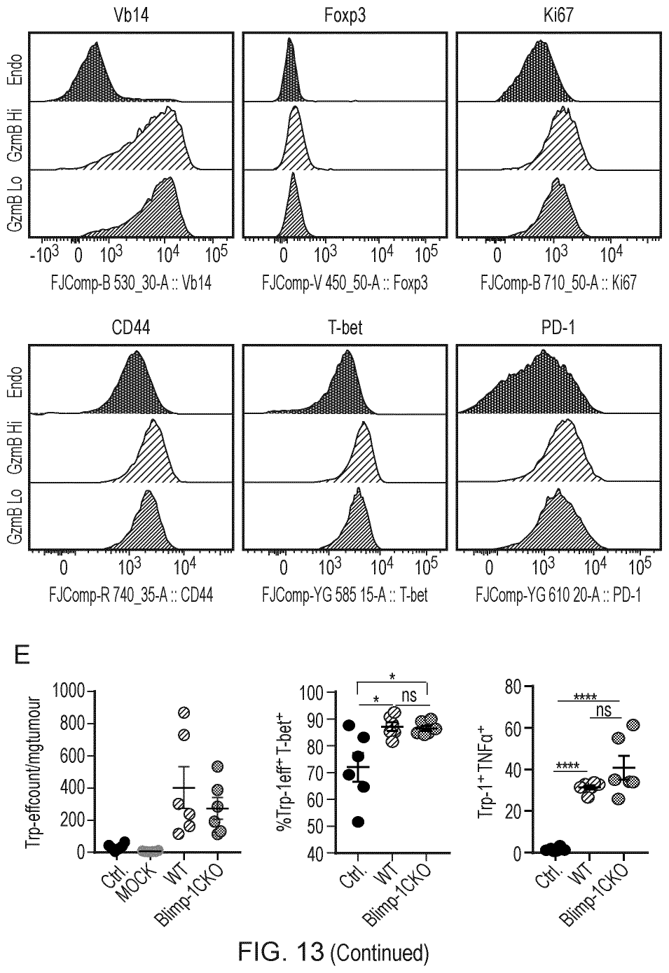

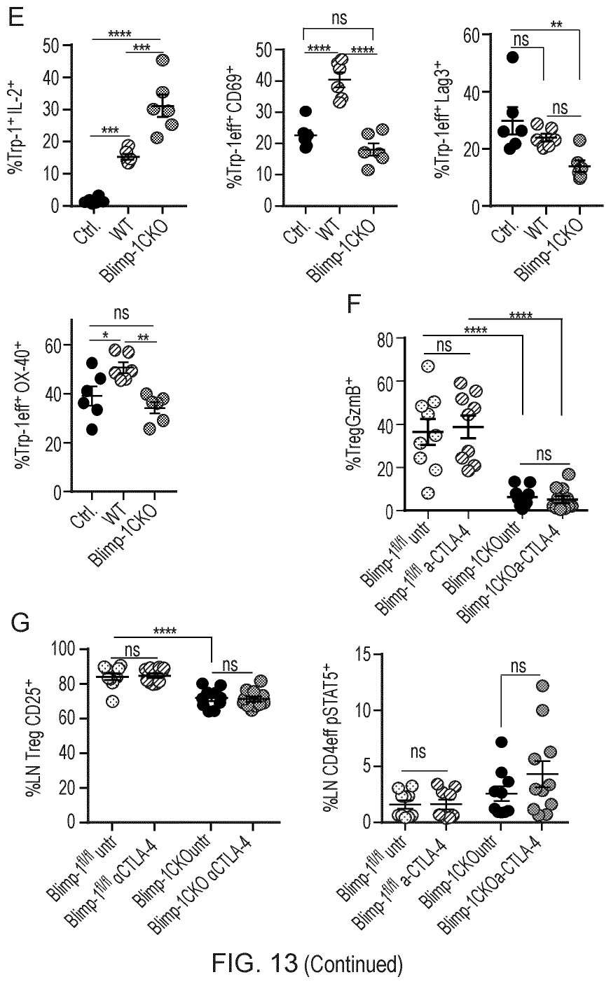

Blimp-1, Bcl-6 and Tcf7 have been shown to be differentially regulated in response to the level of IL-2, with Blimp-1 being upregulated and Bcl-6 downregulated by increasing concentration of IL-2 (Oestreich et al., 2012). Considering that the level of Prdml mRNA was significantly upregulated in CD4⁺ TILS after aCTLA-4 treatment, we further investigated whether Blimp-1 controls the acquisition of a cytotoxic phenotype in CD4 effector cells. Similar to CD8⁺ T cells (Pipkin et al., 2010), Blimp-1-deficient CD4⁺ T cells required a higher concentration of IL-2 than WT cells to upregulate GzmB in vitro. When cultured with a high concentration of IL-2, a similar proportion of WT and Blimp-1 conditional KO (cKO) CD4 cells expressed GzmB but the expression per cell (mean fluorescent intensity; MFI) was lower (FIG. 7A). To investigate whether the IL-2-dependent upregulation of GzmB observed in the tumour was driven by Blimp-1, we first utilized the adoptive transfer model in which activated Trp-1 cells produce high amounts of IL-2. Purified WT and Blimp-1 cKO CD4 T cells were transduced to express the Trp-1 alpha and beta T cell receptor chains. Transduction efficiency was 50% on average, determined by Vb14 receptor staining before the cell transfer (data not shown). Trp1-expressing WT and Blimp-1 cKO CD4⁺ T cells were transferred into B16-bearing mice at day 8 with or without irradiation and aCTLA-4 treatment. As an additional control, mock-transduced WT CD4⁺ T cells were transferred into irradiated WT mice (FIGS. 7B and 13A). Transduced WT Trp-1 CD4⁺ T cells efficiently controlled tumour growth, with kinetics resembling that seen in experiments with naïve non-transduced Trp-1 cells (see FIGS. 1 and 3). Mice that received transduced Blimp-1 cKO CD4⁺ T cells formed slightly larger tumours in comparison to the group that received WT CD4 T cells in short term experiments. Mock transduced polyclonal CD4⁺ T cells failed to control tumour growth (FIG. 13B). The strong autoimmune potential of Bimp-1-deficient CD4⁺ T cells (Martins et al., 2006) made it difficult to evaluate long term impact of Trp-1 Blimp-1 cKO CD4 T cells on tumour growth in adoptive transfer settings.

To better understand how Blimp-1 regulates CD4-T cell mediated anti-tumour response we carried out high-dimensional flow cytometry followed by unsupervised clustering analysis with the PhenoGraph package (Levine et al., 2015) to characterize the landscape of CD4 Trp-1 TILs isolated one week post cell transfer (FIG. 13C). Based on this analysis, we identified several Treg (Foxp3⁺) clusters, a polyclonal CD4⁺ T cell (Vb14-negative) cluster and a Trp-1 (Vb14⁺ Foxp3⁻) cluster, which we divided into GzmB high (clusters 1-2), GzmB medium (cluster 6) and GzmB low (clusters 3-5) (FIG. 7C). WT Trp-1 CD4 T cells were enriched in the GzmB-high cluster whereas Blimp-1-deficient CD4⁺ T cells were enriched in the GzmB low cluster. We further characterized these GzmB clusters and found no difference in Vb14, Ki67, CD44, T-bet or PD-1 expression (FIG. 13D). However, cells enriched in the GzmB-high cluster expressed more CD25, CD69, Lag3, OX-40 and CTLA-4 (FIG. 7D). We confirmed by manual gating analysis that GzmB is expressed at a significantly lower levels by Blimp-1-deficient Trp-1 cells in comparison to WT Trp-1 cells. of relevance, the proportion of CD4 Trp-1 cells expressing TNF□, T-bet and IFNγ was not different between Blimp-1-deficient and WT CD4 Trp-1 cells. In agreement with previous reports, Blimp-1-deficient CD4⁺Trp-1 cells expressed more IL-2 upon restimulation (Martins et al., 2008) (FIGS. 7E and 13E). Together, these data suggest that IL-2− mediated GzmB upregulation by CD4⁺ T cells is at least in part regulated by Blimp-1 in the context of adoptive T cell therapies.

To determine if Blimp-1 is also involved in the acquisition of cytotoxic features by endogenous, non TCR Tg CD4⁺ T cells in the sarcoma model, CD4-Cre Blimp-1 cKO and Blimp-1^{fl/f1} mice were inoculated with MCA205 cells and treated or without aCTLA-4 antibody. In this tumour model, Blimp-1-deficient CD4eff completely lacked GzmB expression, even after aCTLA-4-mediated Treg depletion, whereas expression in CD8⁺ T cells was reduced but not completely lost (FIG. 7F and data not shown). Similar to the adoptive transfer model, T-bet expression was not altered by Blimp-1 deletion in activated CD4⁺ T cells. These data show that CD4⁺ T cells in the tumour microenvironment acquire Th1 characteristics independently of Blimp-1. To exclude the possibility that initial events in the IL-2 signalling pathway are impaired in Blimp-1 deficient CD4⁺ T cells, we assessed STAT5 phosphorylation in Tregs and CD4eff T cells isolated from tumour and LN of a-CTLA-4-treated and untreated mice. Blimp-1-deficient LN Tregs exhibited reduced CD25 expression, while STAT5 phosphorylation was also slightly decreased in comparison to WT cells. However, WT and Blimp-1 cKO CD4 effector TILs in a-CTLA-4 treated tumours exhibited equal levels of CD25 expression and STAT5 phosphorylation (FIGS. 7G and 13G). Thus, although activated Blimp-1 cKO CD4⁺ T cells retain the ability to sense changes in IL-2 levels in the tumour microenvironment, they fail to upregulate GzmB expression. To investigate whether Blimp-1 is required in CD4⁺ T cells for tumour rejection we performed longitudinal studies similar to those performed with T-bet KO mice. In contrast to control mice, Blimp-1 cKO mice did not respond to aCTLA-4 monotherapy (FIG. 7H) demonstrating the critical role of Blimp-1 for both acquisition of cytotoxic activity by CD4⁺ T cells and in vivo tumour control.

### Example 8—Enhanced Expression of GZMB in CD4+ Effector T Cells Treated with a Bcl6 Degrader Compound

Spleens and lymph nodes were taken from C57BL/6J mice, and processed through a 70 μm mesh using PBS, followed by red blood cell lysis using Red Blood cell lysis buffer (Sigma) according to the manufacturer's protocol. Lymphocytes were labelled with CellTrace Violet (ThermoFisher) according to manufacturer's protocol and cultured in RPMI media supplemented with 10% fetal bovine serum, 100 U/mL penicillin, 100 ug/ml streptomycin and 2 mM L-glutamine for 72 h. Cells were either left unstimulated or stimulated with αCD3 (clone 2C11, BioXcell) and αCD28 (clone 37.51, BioXcell) at 0.1 ug/ml. Cells were additionally treated with a Bcl6 degrader (BI-3802) or with a control compound (BI-5273), at a final concentration of 10 nM. The control compound (BI-5273) is a close structural analogue of BI-3802, but has significantly reduced Bcl6 inhibitor activity and therefore acts as a negative control. Cells were stained for flow cytometry using a fixable viability dye (eBiosciences) and with directly conjugated antibodies against CD3, CD4, CD8, FoxP3, CD25, GzmB, PD-1 and Bcl6 using the FoxP3 Transcription factor staining Buffer set (ThermoFisher) according to the manufacturer's instructions.

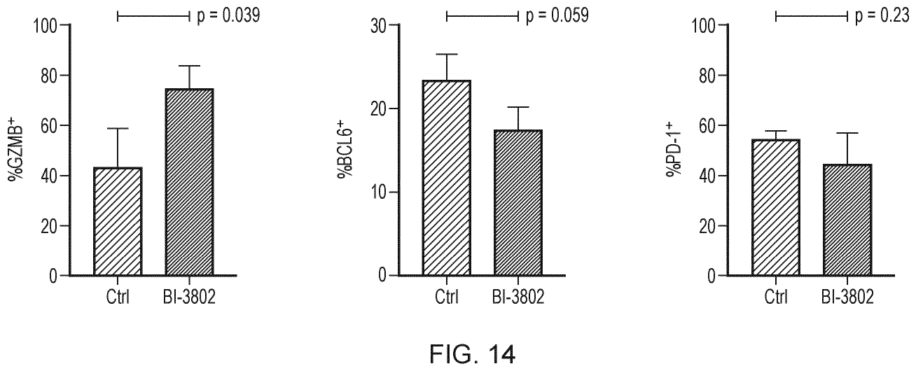

As shown in FIG. 14, a significantly greater percentage of CD4⁺ T Cells treated with the BCL6 targeting drug, BI-3802, express GZMB relative to cells treated with the control compound.

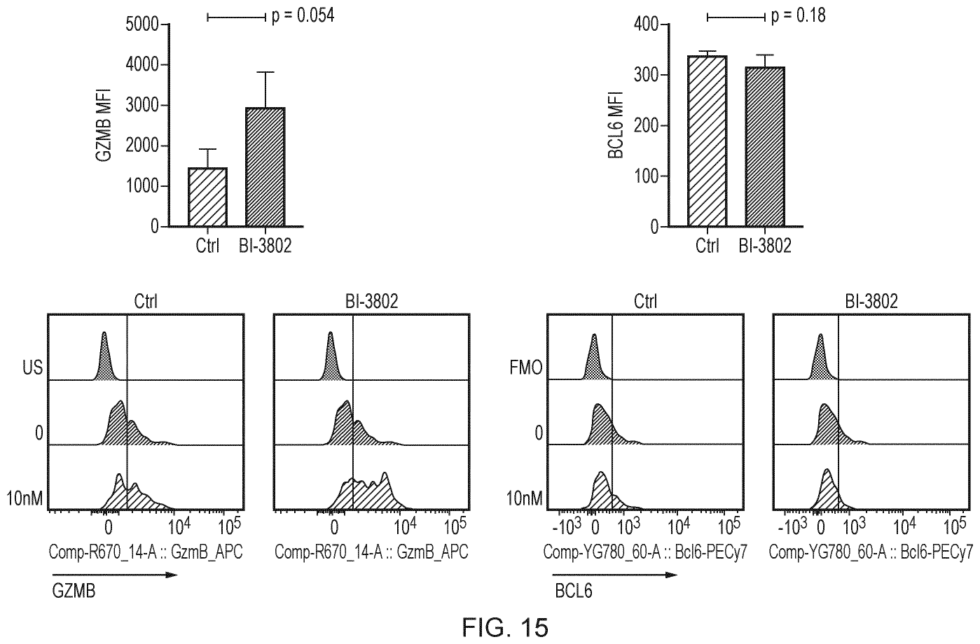

As shown in FIG. 15, cells treated with the BCL6 targeting drug, BI-3802, have increased GZMB expression compared to cells treated with the control compound.

These results show that pharmacological inhibition of Bcl6, like gene knockout of BCL6, increases the anti-cancer therapeutic potential of the T cells as measured by GZMB expression. Enhanced GZMB expression by effector T cells is widely recognised to indicate increased cell killing (e.g. cancer cell killing) activity.

### Example 9—Enhanced Expression of GZMB in T Cells Treated with Further Bcl6 Inhibitors

Healthy donor human PBMCs were labelled with CellTrace Violet (ThermoFisher) according to manufacturer's protocol and cultured in RPMI media supplemented with 10% fetal bovine serum, 100 U/mL penicillin, 100 ug/ml streptomycin and 2 mM L-glutamine for 72 h. Cells were either left unstimulated or stimulated with αCD3 (clone OKT3) at a final concentration of 0.5 ug/ml. Cells were additionally treated with Bcl6 targeting drugs, or with a control compound (BI-5273), at a final concentration of 100 nM. Cells were stained for flow cytometry using a fixable viability dye (eBiosciences) and with directly conjugated antibodies against human CD3, CD4, CD8, FoxP3, CD25, GzmB, and Bcl6 using the FoxP3 Transcription factor staining Buffer set (ThermoFisher) according to the manufacturer's instructions.

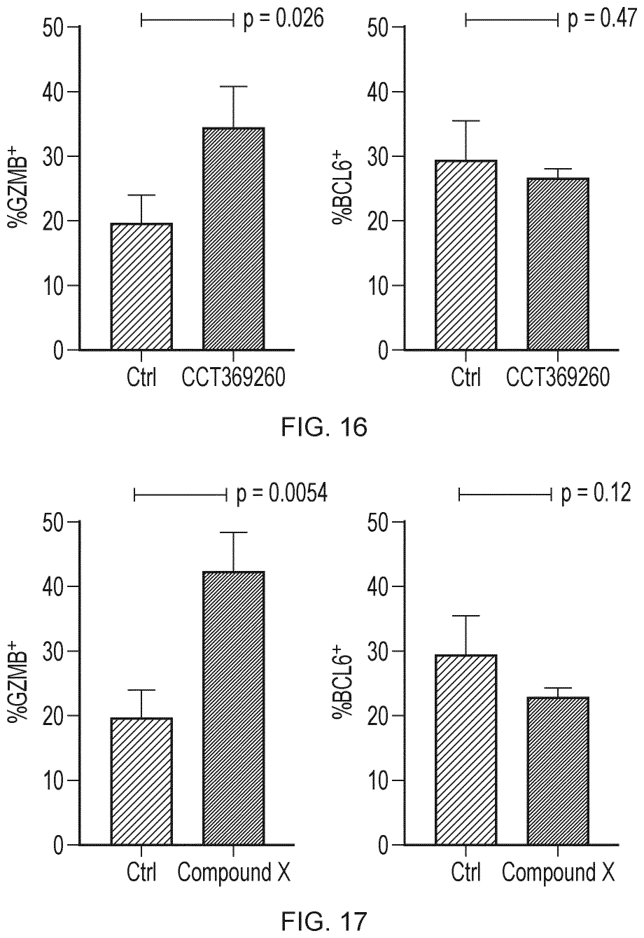

As shown in FIG. 16, a significantly greater percentage of CD4⁺ T Cells treated with the BCL6 targeting drug, CCT369260 (see WO2018/215801), express GZMB relative to cells treated with a control compound, BI-5273.

As shown in FIG. 17, a significantly greater percentage of CD4⁺ T Cells treated with the BCL6 targeting drug, COMPOUND X (disclosed in WO2019/197842), express GZMB relative to cells treated with a control compound, BI-5273.

As shown in FIG. 18, cells treated with the BCL6 targeting drugs CCT369260 (see WO2018/215801) and COMPOUND X (disclosed in WO2019/197842) have increased GZMB expression compared to cells treated with a control compound BI-5273.

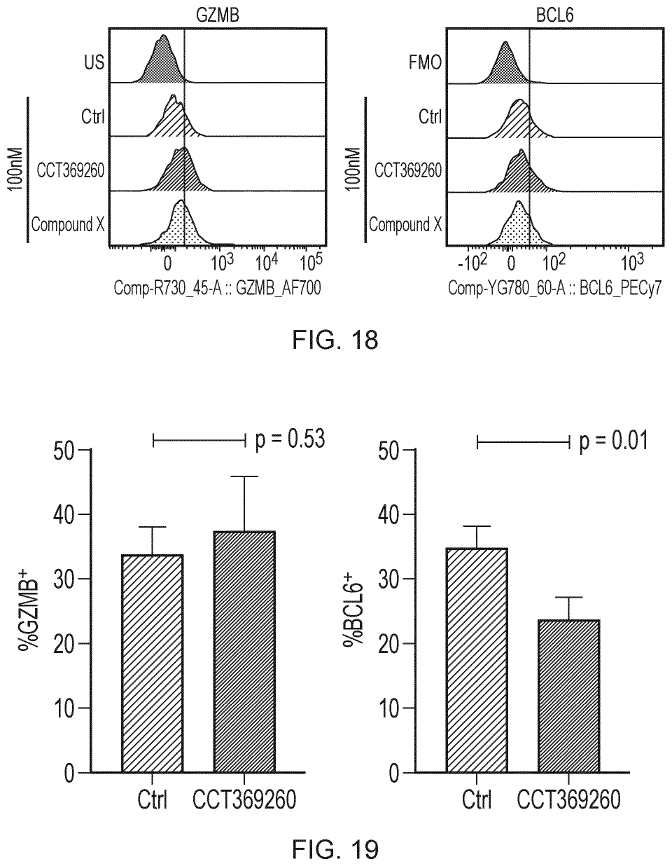

As shown in FIG. 19, a significantly lower percentage of CD8⁺ T Cells treated with the BCL6 targeting drug express BCL6 as compared to the cells treated with control compound. A decrease, although not statistically significant, was seen in the percentage of GZMB positive CD8⁺ cells treated with the BCL6 targeting compound relative to the control compound.

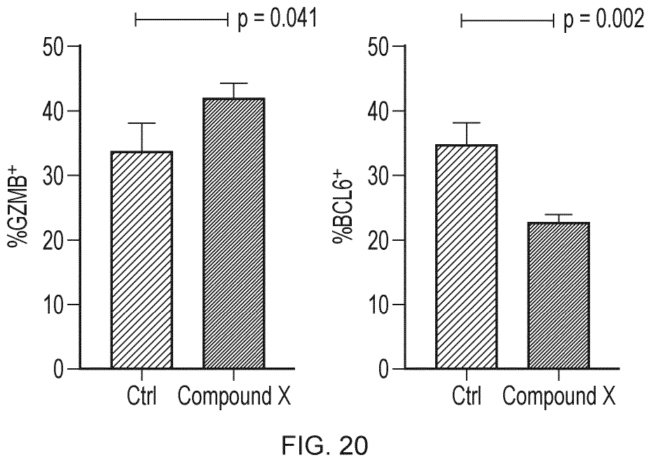

As shown in FIG. 20, a significantly lower percentage of CD8⁺ T Cells treated with the BCL6 targeting drug express BCL6 compared to cells treated with the control compound, which is accompanied by a significant increase in the percentage of CD8⁺ T cells which express GZMB.

These results show that pharmacological inhibition of Bcl6, like gene knockout of BCL6, increases the anti-cancer therapeutic potential of the T cells (both CD4⁺ and CD8⁺) as measured by GZMB expression. Moreover, combined with the results shown in Example 8, it is apparent that the enhanced cell killing effect as assessed by GZMB expression is achieved by a number of different Bcl6 inhibitor compounds. Without wishing to be bound by any particular theory, the present inventors believe that other inhibitors of Bcl6, for example compounds having an IC50 for inhibition of Bcl6 of 1 μM or less, such as 100 nM or less or even 10 nM or less, will likewise augment GZMB expression and thereby enhance cell killing effect (including killing of cancer cells) in T cells, especially CD4⁺ effector T cells. Furthermore, it appears that among Bcl6 inhibitors, Bcl6 degrader compounds exhibit particular efficacy. Consequently, Bcl6 degrader compounds may be preferred Bcl6 inhibitors in accordance with the present invention.

### Example 10—Conditional Knock-Out of BCL6 Gene Enhances the Percentage of GZMB Positive CD4 Positive T Cells in a Mouse Model

### BACKGROUND

Tumour-infiltrating CD4⁺ T cells can acquire cytotoxic potential to give polyfunctional anti-tumour responses.

Cytotoxic CD4⁺ effector T cells are marked by expression of the lytic molecule Granzyme B (GzmB). They also produce helper cytokines IFNγ and TNFα. They have the ability to directly kill infected or transformed cells. Such T cells have been identified in humans and mice. In particular, they have been identified in the PBMCs of humans with chronic viral infections, including hepatitis viruses as well as in human cancers, including CLL. In the murine B16 melanoma model, adoptive therapy of cytotoxic CD4⁺ T cells leads to complete tumour rejection.

Methodology

Use of Bcl6 cKO mice to determine the role of Bcl6 in acquisition of cytotoxic phenotype in CD4⁺ T cells.

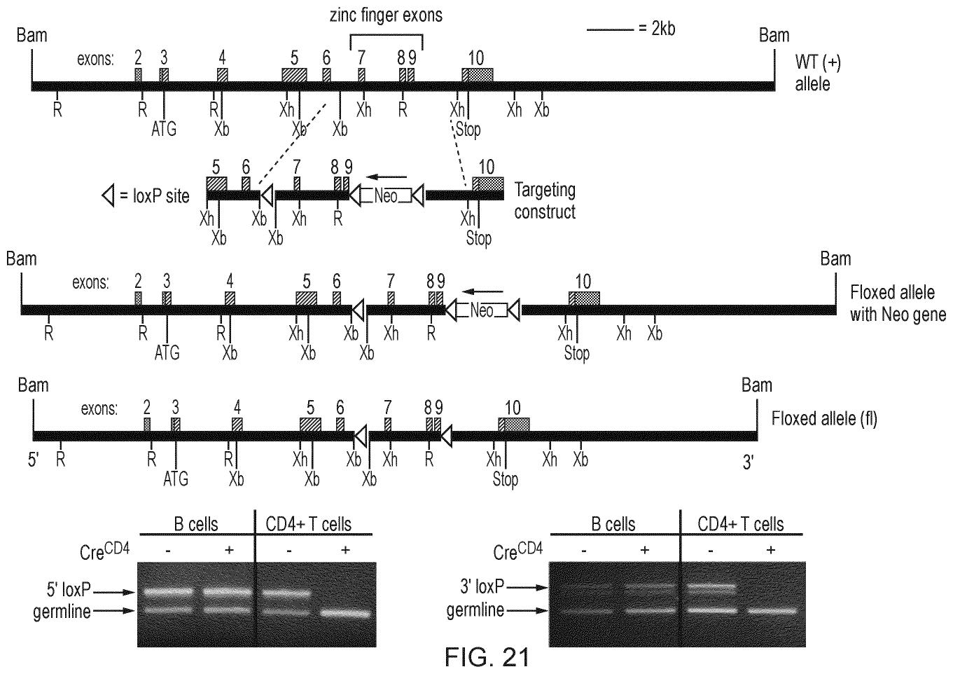

Development of a Bcl6 conditional knock-out (KO) is shown in FIG. 21. As can been seen exons 7, 8 and 9 are flanked by loxP sites. When CD4Cre is present, T cells lack Bcl6. From Hollister et al., J Immunol 2013. Bcl6^{fl/f1} Mice were purchased from JAX, and bred to CD4Cre mice.

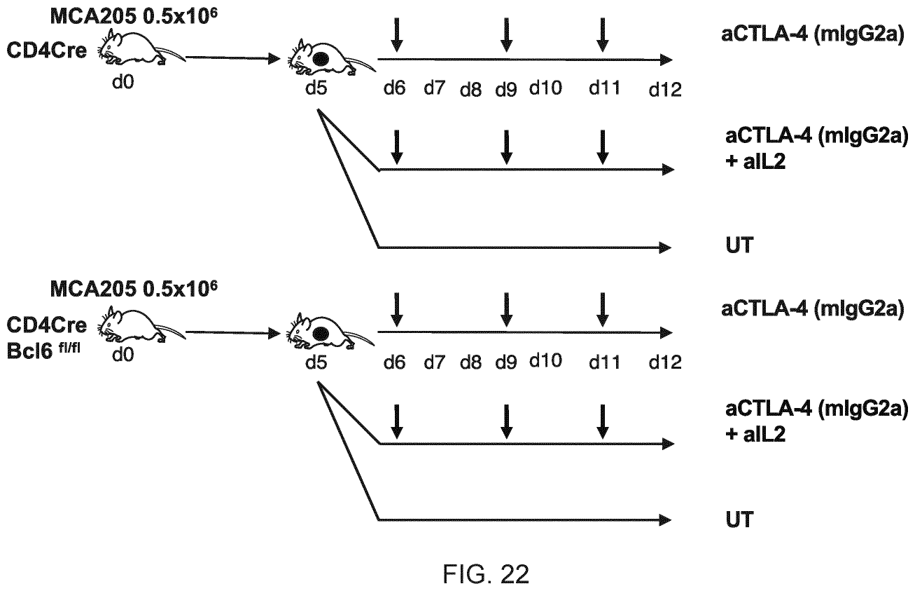

FIG. 22 shows a schematic representation of the experimental setup, using CD4Cre (Ctrl) and CD4Cre Bcl6^{fl/f1} mice. Mice are injected subcutaneously with 0.5×10⁶ MCA205 tumour cells and treated with aCTLA4 or aCTLA4+aIL2 on days 6, 9 and 11 after tumour inoculation.

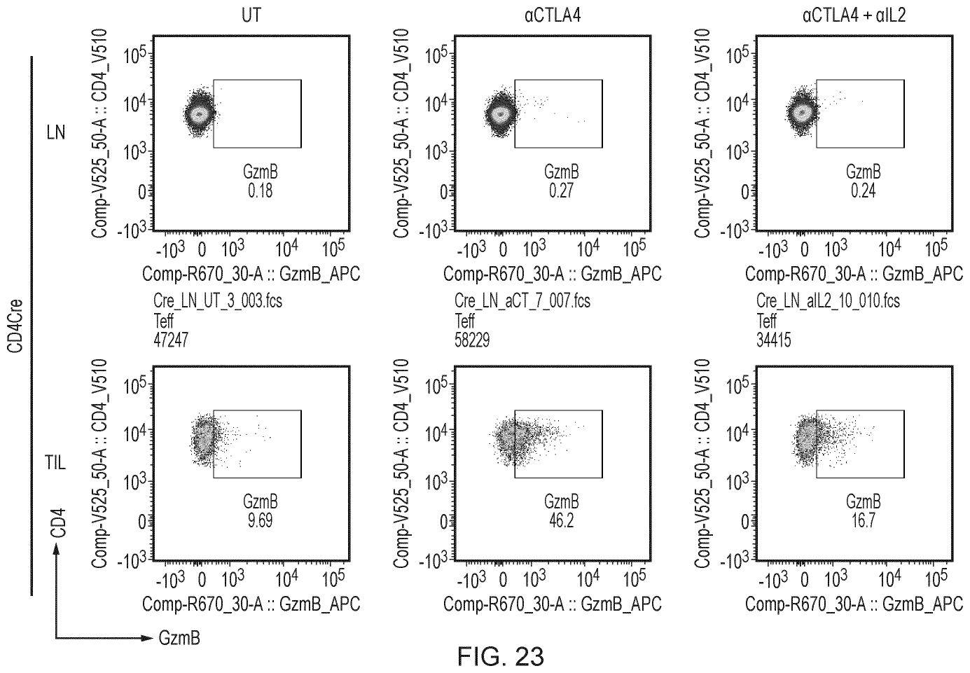

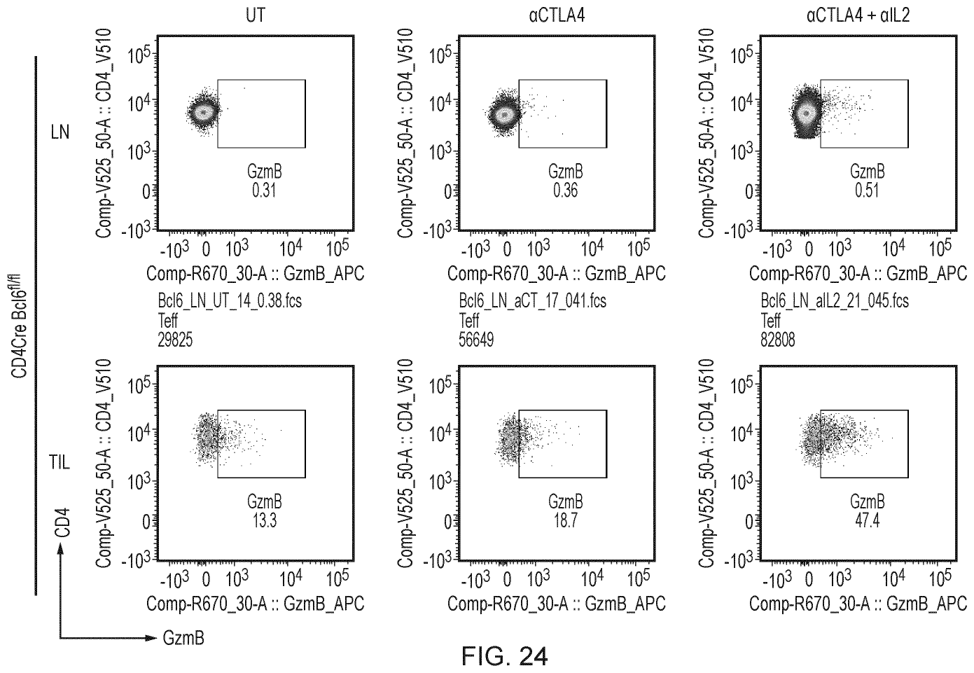

As shown in FIGS. 23 and 24, respectively, a higher percentage of Bcl6-deficient TILs express GzmB.

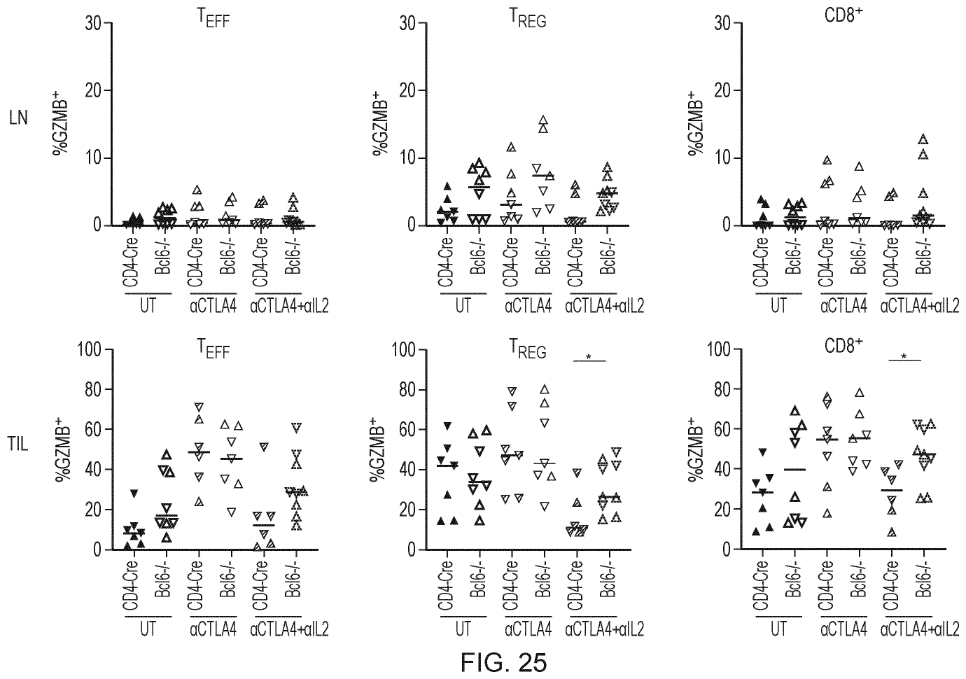

As shown in FIG. 25, a higher percentage of Bcl6-deficient TILs express GzmB.

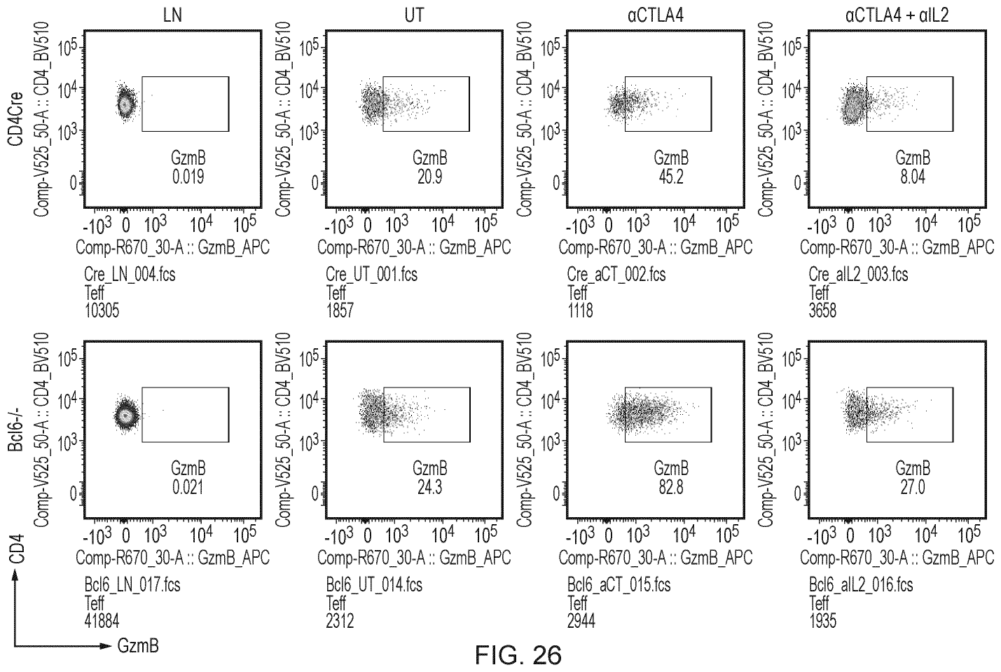

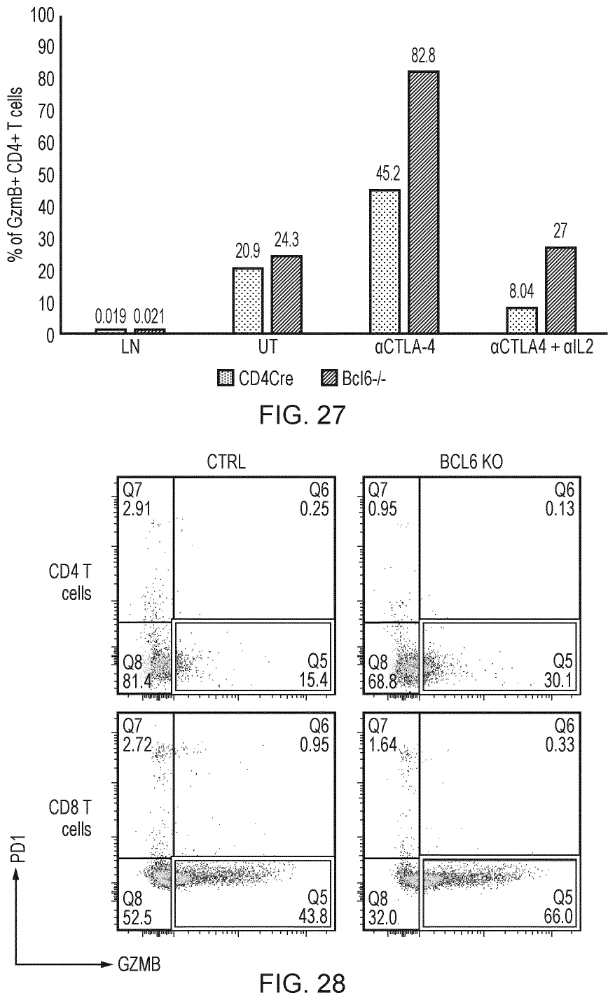

FIG. 26 shows a representative flow plots showing GzmB expression by CD4⁺ Teff cells from tumours and draining LN of CD4Cre mice and CD4Cre Bcl6fl/fl mice. The flow plots are quantified in FIG. 27 which shows that that Bcl6 knock-out mice displayed higher percentage of GzmB positive CD4⁺ T cells.

These results demonstrate that inhibition of Bcl6 via functional inhibitors or by gene knock out of silencing can be expected to enhance cell killing activity of T cells, especially CD4⁺ effector T cells.

### Example 11—CRISPR Gene Editing to Knock-Out BCL6 Expression in Human PBMCs Results in Enhanced GZMB Percentage in Both CD4⁺ and CD8⁺ T Cells

Gene-editing of human T cells using CRISPR/Cas9 technology.

Frozen peripheral blood mononuclear cells are thawed and incubated on overnight in as 12-well plate (Cat. No 353043, Falcon) containing 2 mL of growth media (RPMI 1640 (Cat. No 51536C, Sigma-Aldrich) supplemented with 5 mL of Penicilin-Streptomycin (Cat. No P4333, Sigma-Aldrich) and 50 mL of Human Serum (10% final conc) (Cat: G7513-100 mL, Sigma-Aldrich)) supplemented with 50 IU/mL of IL2 (Proleukin, Novartis). The following day, cells are transferred into a 12-well plate coated with 10 μg/mL of αCD3 antibody (Cat No. BE0001-2, BioXcell, Clone: OKT3) and cultured in 2 mL of growth media containing 100 IU/mL of IL2 and 10 μg/mL of αCD28 antibody (Cat. No BE0248, BioXcell, Clone: 9.3) for 72 hours. The sgRNA is prepared by mixing the Alt-R tracrRNA(Cat. No 1072534 IDT) and the Alt-R crRNA 200 μM (custom-made, IDT) in a 0.6 mL tube and incubated 5 minutes at 95 C. The sgRNA is mixed with the Nuclease-Free Duplex Buffer (Cat. No 11050112, IDT) and then mixed with the Cas9 protein (Cat. No 1081059 IDT) to form the ribonucleoprotein complex. Cells are electroporated with the ribonucleoprotein complex (For 4D-Nucleofector X Kit Small (V4XP-3032 Lonza) following the manufacturer protocol and left on growth media containing 100 IU/mL for three days. Finally, cells are stained for flow cytometry and the knock-out of the target gene is measured by quantifying the number of cells positive for the target relative to the number of CD4 or CD8 T cells. The sequence of the crRNA for BCL6 knock-out was:

CAGTCAAGATGTCTCGACTC (SEQ ID NO: 1)

Human peripheral blood mononuclear cells, were stimulated for three days using αCD3 and αCD28 antibodies. On day three, cells were electroporated with the Cas9 protein and with the crRNA targeting BCL6 (SEQ ID NO: 1). Cells were kept in culture for 10 days using low doses of interleukin-2. On day 10 cells were stained with cell trace violet and restimulated for four days with a low dose of dynabeads containing αCD3 and αCD28. On day 12, cells were incubated with brefeldin A for four hours in order to accumulate cytokines. Cells were stained for flow cytometry and acquired in the FACS symphony.

The flow cytometry results are shown in FIG. 28. The Left panels show representative panels of GZMB versus PD1 in both CD4 (upper) and CD8 (lower) control T cells. The right panels show representative panels of GZMB versus PD1 in both CD4 (upper) and CD8 (lower) BCL6 KO T cells

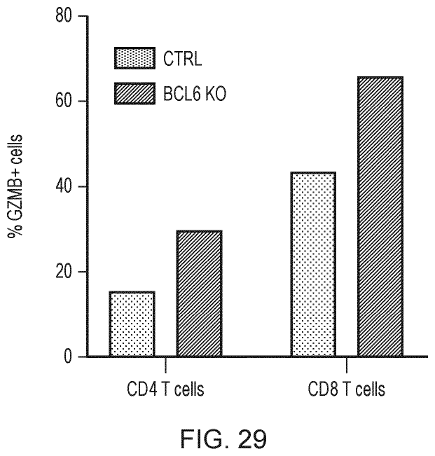

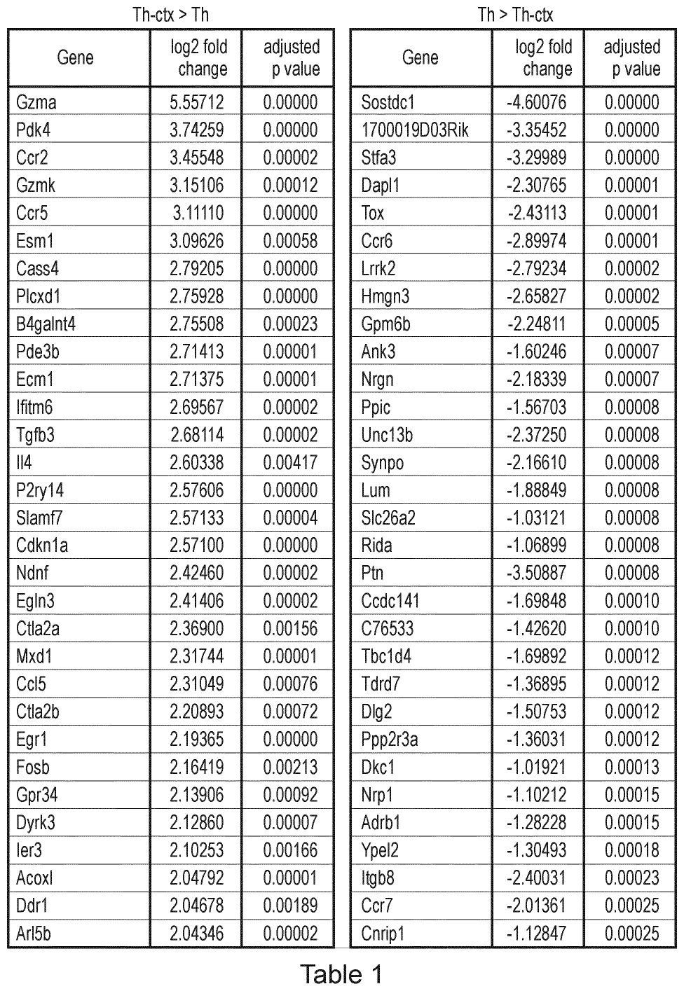

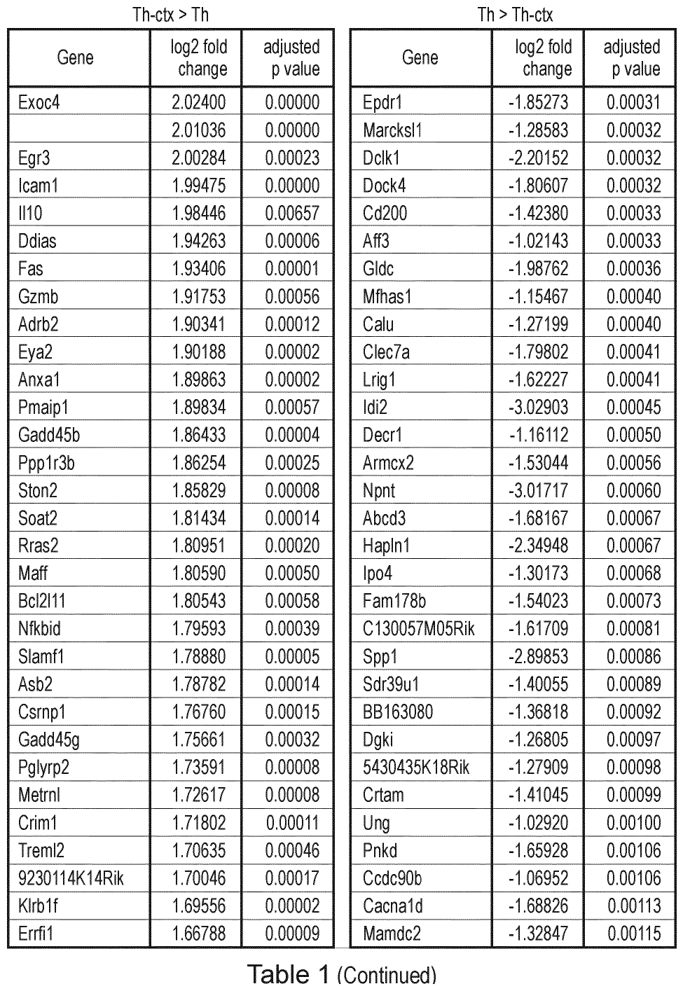

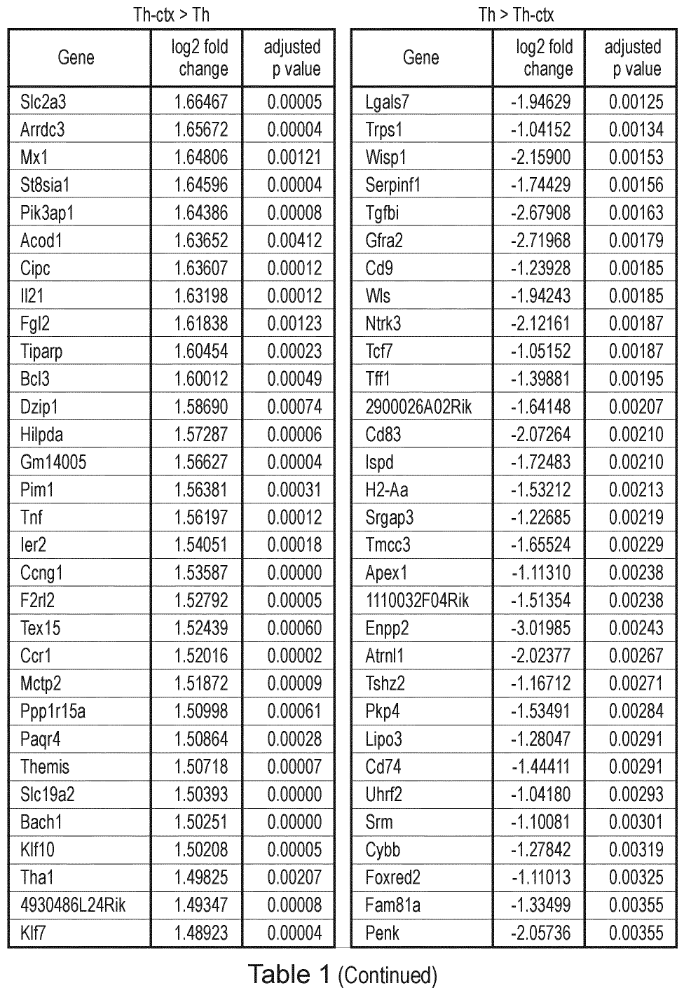

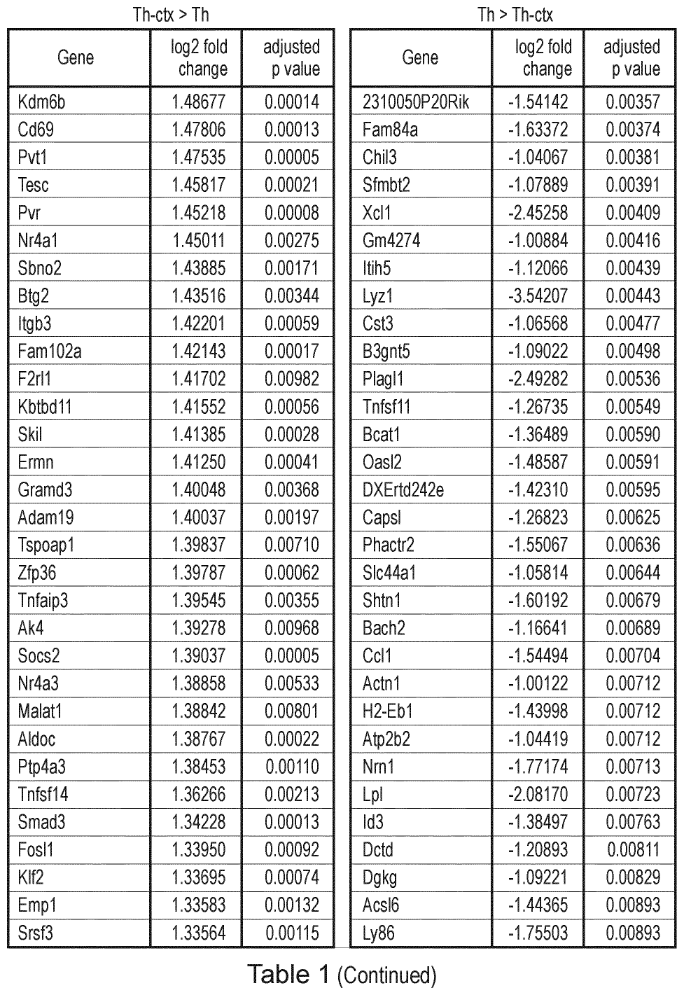

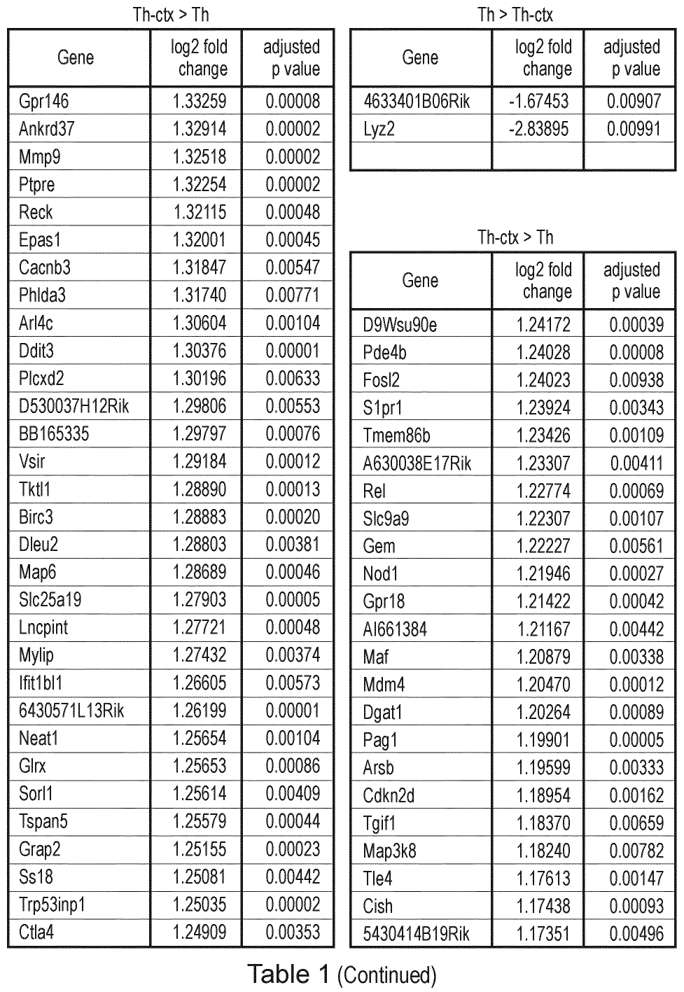

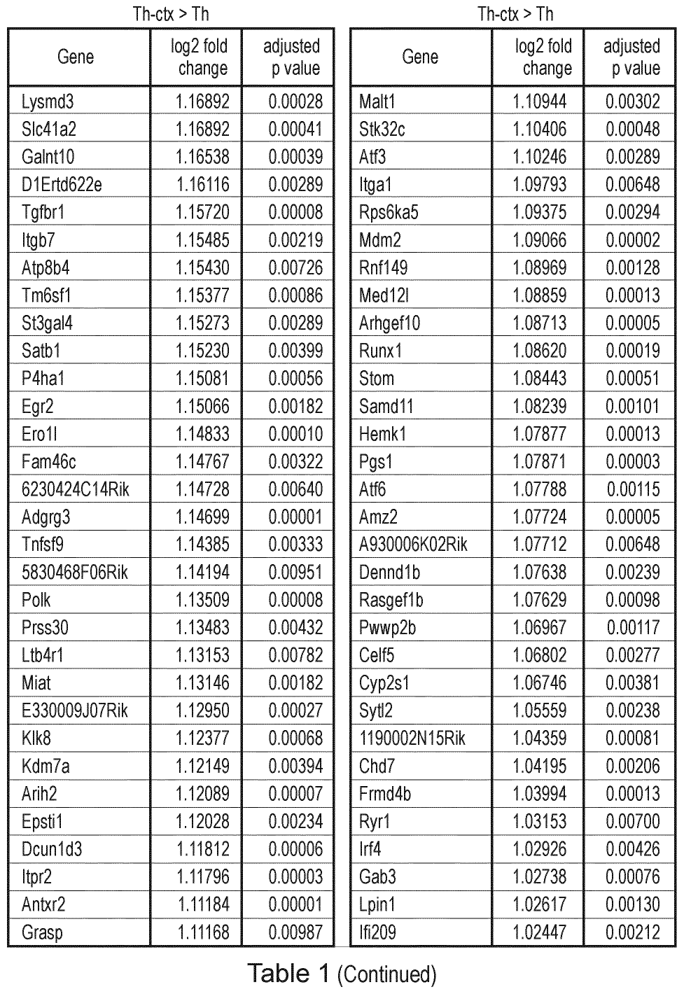

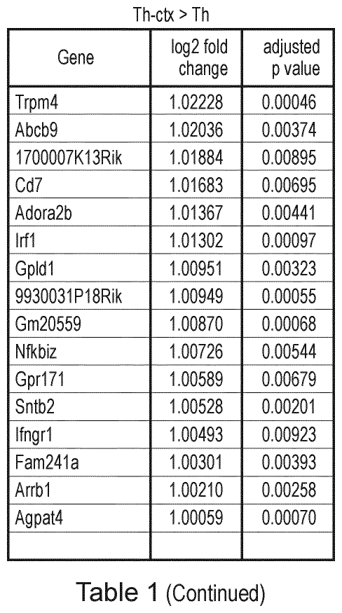

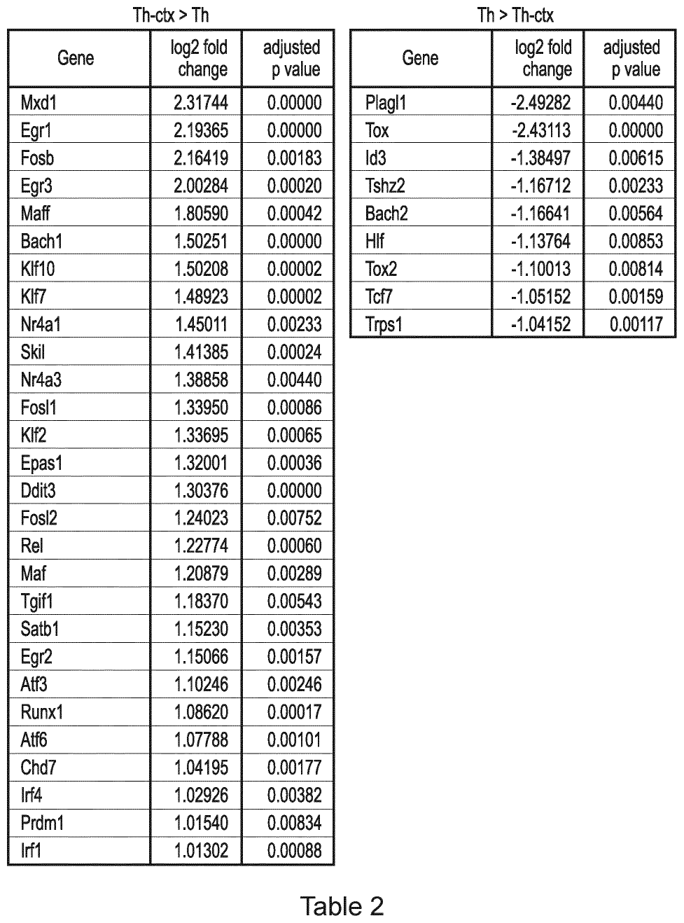

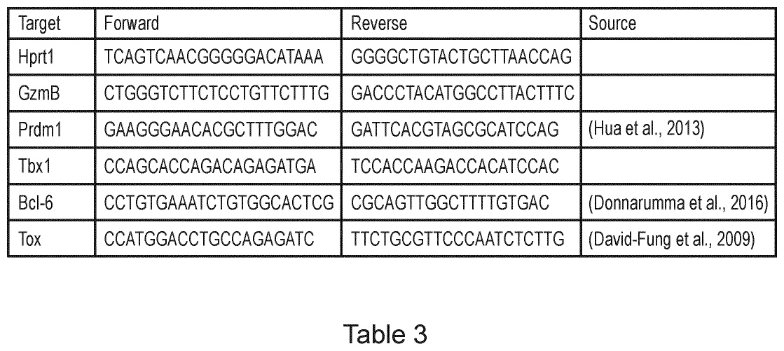

The results of the flow cytometry experiment shown in FIG. 28 were quantified and are shown in the bar graph of FIG. 29. It is clearly apparent that knock-out of BCL6 gene in the human cells resulted in a greater percentage of GZMB positive cells among both the CD4⁺ compartment and the CD8⁺ compartment. In agreement with the mouse conditional knock-out results and the pharmacological inhibitor studies described above, these results demonstrate that BCL6 downregulation results in greater capacity for cancer cell killing by increasing the percentage of T cells that have the anti-cancer cell phenotype.

All references cited herein are incorporated herein by reference in their entirety and for all purposes to the same extent as if each individual publication or patent or patent application was specifically and individually indicated to be incorporated by reference in its entirety.

The specific embodiments described herein are offered by way of example, not by way of limitation. Any sub-titles herein are included for convenience only, and are not to be construed as limiting the disclosure in any way.

### REFERENCES

Appay, V., Zaunders, J. J., Papagno, L., Sutton, J., Jaramillo, A., Waters, A., Easterbrook, P., Grey, P., Smith, D., McMichael, A. J., et al. (2002). Characterization of CD4+CTLs Ex Vivo. J. Immunol. 168, 5954-5958.
Arce Vargas, F., Furness, A. J. S., Litchfield, K., Joshi, K., Rosenthal, R., Ghorani, E., Solomon, I., Lesko, M. H., Ruef, N., Roddie, C., et al. (2018). Fc Effector Function Contributes to the Activity of Human Anti-CTLA-4 Antibodies. Cancer Cell 33, 649-663.e4.
Ballesteros-Tato, A., León, B., Graf, B. A., Moquin, A., Adams, P. S., Lund, F. E., and Randall, T. D. (2012). Interleukin-2 inhibits germinal center formation by limiting T follicular helper cell differentiation. Immunity 36, 847-856.
Bankoti, R., Ogawa, C., Nguyen, T., Emadi, L., Couse, M., Salehi, S., Fan, X., Dhall, D., Wang, Y., Brown, J., et al. (2017). Differential regulation of Effector and Regulatory T cell function by Blimp1. Sci. Rep. 7, 1-14.
Boyman, O., and Sprent, J. (2012). The role of interleukin-2 during homeostasis and activation of the immune system. Nat. Rev. Immunol. 12, 180-190.
Brown, D. M., Kamperschroer, C., Dilzer, A. M., Roberts, D. M., and Swain, S. L. (2009). IL-2 and antigen dose differentially regulate perforin- and FasL-mediated cytolytic activity in antigen specific CD4+ T cells. Cell. Immunol. 257, 69-79.
Castro, I., Dee, M. J., and Malek, T. R. (2012). Transient Enhanced IL-2R Signaling Early during Priming Rapidly Amplifies Development of Functional CD8+T Effector-Memory Cells. J. Immunol. 189, 4321-4330.
Chihara, N., Madi, A., Kondo, T., Zhang, H., Acharya, N., Singer, M., Nyman, J., Marjanovic, N. D., Kowalczyk, M. S., Wang, C., et al. (2018). Induction and transcriptional regulation of the co-inhibitory gene module in T cells. Nature 558, 454-459.
Choi, Y. S., Gullicksrud, J. A., Xing, S., Zeng, Z., Shan, Q., Li, F., Love, P. E., Peng, W., Xue, H. H., and Crotty, S. (2015). LEF-1 and TCF-1 orchestrate TFHdifferentiation by regulating differentiation circuits upstream of the transcriptional repressor Bcl6. Nat. Immunol. 16, 980-990.
David-Fung, E.-S., Butler, R., Buzi, G., Yui, M. A., Diamond, R. A., Anderson, M. K., Rowen, L., and Rothenberg, E. V. (2009). Transcription factor expression dynamics of early T-lymphocyte specification and commitment. Dev. Biol. 325, 444-467.
DiToro, D., Winstead, C. J., Pham, D., Witte, S., Andargachew, R., Singer, J. R., Wilson, C. G., Zindl, C. L., Luther, R. J., Silberger, D. J., et al. (2018). Differential IL-2 expression defines developmental fates of follicular versus nonfollicular helper T cells. Science (80-. ). 361, eaao2933.
Donnarumma, T., Young, G. R., Merkenschlager, J., Eksmond, U., Bongard, N., Nutt, S. L., Boyer, C., Dittmer, U., Le-Trilling, V. T. K., Trilling, M., et al. (2016). Opposing Development of Cytotoxic and Follicular Helper CD4 T Cells Controlled by the TCF-1-Bcl6 Nexus. Cell Rep. 17, 1571-1583.
Dupage, M., and Bluestone, J. A. (2016). Harnessing the plasticity of CD4+ T cells to treat immune-mediated disease. Nat. Rev. Immunol. 16, 149-163.
Eshima, K., Misawa, K., Ohashi, C., and Iwabuchi, K. (2018). Role of T-bet, the master regulator of Th1 cells, in the cytotoxicity of murine CD4+ T cells. Microbiol. Immunol. 62, 348-356.
Evans, C. M., and Jenner, R. G. (2013). Transcription factor interplay in T helper cell differentiation. Brief. Funct. Genomics 12, 499-511.
Fu, S. H., Yeh, L. T., Chu, C. C., Yen, B. L. J., and Sytwu, H. K. (2017). New insights into Blimp-1 in T lymphocytes: A divergent regulator of cell destiny and effector function. J. Biomed. Sci. 24, 1-17.
Gattinoni, L., Finkelstein, S. E., Klebanoff, C. A., Antony, P. A., Palmer, D. C., Spiess, P. J., Hwang, L. N., Yu, Z., Wrzesinski, C., Heimann, D. M., et al. (2005). Removal of homeostatic cytokine sinks by lymphodepletion enhances the efficacy of adoptively transferred tumor-specific CD8+ T cells. J. Exp. Med. 202, 907-912.
Gerstberger, S., Hafner, M., and Tuschl, T. (2014). A census of human RNA-binding proteins. Nat. Rev. Genet. 15, 829-845.
Glimcher, L. H., Townsend, M. J., Sullivan, B. M., and Lord, G. M. (2004). Recent developments in the transcriptional regulation of cytolytic effector cells. Nat. Rev. Immunol. 4, 900-911.
Godec, J., Tan, Y., Liberzon, A., Tamayo, P., Bhattacharya, S., Butte, A. J., Mesirov, J. P., and Haining, W. N. (2016). Compendium of Immune Signatures Identifies Conserved and Species-Specific Biology in Response to Inflammation. Immunity 44, 194-206.
Haigh, T. a., Lin, X., Jia, H., Hui, E. P., Chan, a. T. C., Rickinson, a. B., and Taylor, G. S. (2008). EBV Latent Membrane Proteins (LMPs) 1 and 2 as Immunotherapeutic Targets: LMP-Specific CD4+ Cytotoxic T Cell Recognition of EBV-Transformed B Cell Lines. J. Immunol. 180, 1643-1654.
Hannani, D., Vétizou, M., Enot, D., Rusakiewicz, S., Chaput, N., Klatzmann, D., Desbois, M., Jacquelot, N., Vimond, N., Chouaib, S., et al. (2015). Anticancer immunotherapy by CTLA-4 blockade: Obligatory contribution of IL-2 receptors and negative prognostic impact of soluble CD25. Cell Res. 25, 208-224.
Hotblack, A., Holler, A., Piapi, A., Ward, S., Stauss, H. J., and Bennett, C. L. (2018). Tumor-Resident Dendritic Cells and Macrophages Modulate the Accumulation of TCR-Engineered T Cells in Melanoma. Mol. Ther. 26, 1471-1481.
Hua, L., Yao, S., Pham, D., Jiang, L., Wright, J., Sawant, D., Dent, A. L., Braciale, T. J., Kaplan, M. H., and Sun, J. (2013). Cytokine-Dependent Induction of CD4+ T cells with Cytotoxic Potential during Influenza Virus Infection. J. Virol. 87, 11884-11893.
Irizarry, R. A. (2003). Exploration, normalization, and summaries of high density oligonucleotide array probe level data. Biostatistics 4, 249-264.
Janas, M. L., Groves, P., Kienzle, N., and Kelso, A. (2005). IL-2 Regulates Perforin and Granzyme Gene Expression in CD8+ T Cells Independently of Its Effects on Survival and Proliferation. J. Immunol. 175, 8003-8010.
Johnston, R. J., Poholek, A. C., DiToro, D., Yusuf, I., Eto, D., Barnett, B., Dent, A. L., Craft, J., and Crotty, S. (2009). Bcl6 and Blimp-1 Are Reciprocal and Antagonistic Regulators of T Follicular Helper Cell Differentiation. Science (80-. ). 325, 1006-1010.
Juno, J. A., van Bockel, D., Kent, S. J., Kelleher, A. D., Zaunders, J. J., and Munier, C. M. L. (2017). Cytotoxic CD4 T Cells-Friend or Foe during Viral Infection? Front. Immunol. 8, 19.
Kanhere, A., Hertweck, A., Bhatia, U., Gökmen, M. R., Perucha, E., Jackson, I., Lord, G. M., and Jenner, R. G. (2012). T-bet and GATA3 orchestrate Th1 and Th2 differentiation through lineage-specific targeting of distal regulatory elements. Nat. Commun. 3, 1268.
Kim, J. M., Rasmussen, J. P., and Rudensky, A. Y. (2007). Regulatory T cells prevent catastrophic autoimmunity throughout the lifespan of mice. Nat. Immunol. 8, 191-197.
Kitano, S., Tsuji, T., Liu, C., Hirschhorn-Cymerman, D., Kyi, C., Mu, Z., Allison, J. P., Gnjatic, S., Yuan, J. D., and Wolchok, J. D. (2013). Enhancement of tumor-reactive cytotoxic CD4⁺ T cell responses after ipilimumab treatment in four advanced melanoma patients. Cancer Immunol. Res. 1, 235-244.
Lee, J.-Y., Skon, C. N., Lee, Y. J., Oh, S., Taylor, J. J., Malhotra, D., Jenkins, M. K., Rosenfeld, M. G., Hogquist, K. A., and Jameson, S. C. (2015). The Transcription Factor KLF2 Restrains CD4+T Follicular Helper Cell Differentiation. Immunity 42, 252-264.
Levanon, D., Negreanu, V., Lotem, J., Bone, K. R., Brenner, O., Leshkowitz, D., and Groner, Y. (2014). Transcription factor Runx3 regulates interleukin-15-dependent natural killer cell activation. Mol. Cell. Biol. 34, 1158-1169.
Levine, J. H., Simonds, E. F., Bendall, S. C., Davis, K. L., Amir, E. D., Tadmor, M. D., Litvin, O., Fienberg, H. G., Jager, A., Zunder, E. R., et al. (2015). Data-Driven Phenotypic Dissection of AML Reveals Progenitor-like Cells that Correlate with Prognosis. Cell 162, 184-197.
Li, Q., Rao, R. R., Araki, K., Pollizzi, K., Odunsi, K., Powell, J. D., and Shrikant, P. A. (2011). A Central Role for mTOR Kinase in Homeostatic Proliferation Induced CD8+ T Cell Memory and Tumor Immunity. Immunity 34, 541-553.
Lupar, E., Brack, M., Garnier, L., Laffont, S., Rauch, K. S., Schachtrup, K., Arnold, S. J., Guéry, J.-C., and Izcue, A. (2015). Eomesodermin Expression in CD4+ T Cells Restricts Peripheral Foxp3 Induction. J. Immunol. 195, 4742-4752.
Marnik, E. A., Wang, X., Sproule, T. J., Park, G., Christianson, G. J., Lane-Reticker, S. K., Jain, S., Duffy, T., Wang, H., Carter, G. W., et al. (2017). Precocious Interleukin 21 Expression in Naive Mice Identifies a Natural Helper Cell Population in Autoimmune Disease. Cell Rep. 21, 208-221.
Martins, G. A., Cimmino, L., Shapiro-Shelef, M., Szabolcs, M., Herron, A., Magnusdottir, E., and Calame, K. (2006). Transcriptional repressor Blimp-1 regulates T cell homeostasis and function. Nat. Immunol. 7, 457-465.
Martins, G. A., Cimmino, L., Liao, J., Magnusdottir, E., and Calame, K. (2008). Blimp-1 directly represses 112 and the 112 activator Fos, attenuating T cell proliferation and survival. J. Exp. Med. 205, 1959-1965.
Michael McNamara (2014). No Title. J. Immunother. Cancer. Mosmann, T. R., Cherwinski, H., Bond, M. W., Giedlin, M. A., and Coffman, R. L. (1986). Two types of murine helper T cell clone. I. Definition according to profiles of lymphokine activities and secreted proteins. J. Immunol. 136, 2348-2357.
Mucida, D., Husain, M. M., Muroi, S., Van Wijk, F., Shinnakasu, R., Naoe, Y., Reis, B. S., Huang, Y., Lambolez, F., Docherty, M., et al. (2013). Transcriptional reprogramming of mature CD4⁺ helper T cells generates distinct MHC class II-restricted cytotoxic T lymphocytes. Nat. Immunol. 14, 281-289.
Muranski, P., Boni, A., Antony, P. a, Cassard, L., Irvine, K. R., Kaiser, A., Paulos, C. M., Palmer, D. C., Touloukian, C. E., Ptak, K., et al. (2008). Tumor-specific Th17-polarized cells eradicate large established melanoma. Blood 112, 362-373.
Mustafa, A. S., and Godal, T. (1987). BCG induced CD4+ cytotoxic T cells from BCG vaccinated healthy subjects: relation between cytotoxicity and suppression in vitro. Clin. Exp. Immunol. 69, 255-262.
Oestreich, K. J., Mohn, S. E., and Weinmann, A. S. (2012). Molecular mechanisms that control the expression and activity of Bcl-6 in TH1 cells to regulate flexibility with a TFH-like gene profile. Nat. Immunol. 13, 405-411.
Oja, A. E., Vieira Braga, F. A., Remmerswaal, E. B. M., Kragten, N. A. M., Hertoghs, K. M. L., Zuo, J., Moss, P. A., van Lier, R. A. W., van Gisbergen, K. P. J. M., and Hombrink, P. (2017). The Transcription Factor Hobit Identifies Human Cytotoxic CD4+ T Cells. Front. Immunol. 8, 325.
Ottenhoff, T. H. (1988). The recombinant 65-kD heat shock protein of Mycobacterium bovis Bacillus Calmette-Guerin/M. tuberculosis is a target molecule for CD4+ cytotoxic T lymphocytes that lyse human monocytes. J. Exp. Med. 168, 1947-1952.
Peggs, K. S., Quezada, S. A., Chambers, C. A., Korman, A. J., and Allison, J. P. (2009). Blockade of CTLA-4 on both effector and regulatory T cell compartments contributes to the antitumor activity of anti-CTLA-4 antibodies. J. Exp. Med. 206, 1717-1725.
Péguillet, I., Milder, M., Louis, D., Vincent-Salomon, A., Dorval, T., Piperno-Neumann, S., Scholl, S. M., and Lantz, O. (2014). High Numbers of Differentiated Effector CD4 T Cells Are Found in Patients with Cancer and Correlate with Clinical Response after Neoadjuvant Therapy of Breast Cancer. Cancer Res. 74, 2204-2216.
Pipkin, M. E., Sacks, J. A., Cruz-Guilloty, F., Lichtenheld, M. G., Bevan, M. J., and Rao, A. (2010). Interleukin-2 and inflammation induce distinct transcriptional programs that promote the differentiation of effector cytolytic T cells. Immunity 32, 79-90.
Quezada, S. a, Simpson, T. R., Peggs, K. S., Merghoub, T., Vider, J., Fan, X., Blasberg, R., Yagita, H., Muranski, P., Antony, P. a, et al. (2010). Tumor-reactive CD4(+) T cells develop cytotoxic activity and eradicate large established melanoma after transfer into lymphopenic hosts. J. Exp. Med. 207, 637-650.
Quezada, S. A., Peggs, K. S., Curran, M. A., and Allison, J. P. (2006). CTLA4 blockade and GM-CSF combination immunotherapy alters the intratumor balance of effector and regulatory T cells. J. Clin. Invest. 116, 1935-1945.
Qui, H. Z., Hagymasi, A. T., Bandyopadhyay, S., St. Rose, M.-C., Ramanarasimhaiah, R., Menoret, A., Mittler, R. S., Gordon, S. M., Reiner, S. L., Vella, A. T., et al. (2011). CD134 Plus CD137 Dual Costimulation Induces Eomesodermin in CD4 T Cells To Program Cytotoxic Th1 Differentiation. J. Immunol. 187, 3555-3564.
Sakaguchi, S., Yamaguchi, T., Nomura, T., and Ono, M. (2008). Regulatory T Cells and Immune Tolerance. Cell 133, 775-787.
Schey, R., Dornhoff, H., Baier, J. L. C., Purtak, M., Opoka, R., Koller, A. K., Atreya, R., Rau, T. T., Daniel, C., Amann, K., et al. (2016). CD101 inhibits the expansion of colitogenic T cells. Mucosal Immunol. 9, 1205-1217.
Selby, M. J., Engelhardt, J. J., Quigley, M., Henning, K. A., Chen, T., Srinivasan, M., and Korman, A. J. (2013). Anti-CTLA-4 Antibodies of IgG2a Isotype Enhance Antitumor Activity through Reduction of Intratumoral Regulatory T Cells. Cancer Immunol. Res. 1, 32-42.
Sergushichev, A. (2016). An algorithm for fast preranked gene set enrichment analysis using cumulative statistic calculation. BioRxiv 060012.
Serroukh, Y., Gu-Trantien, C., Hooshiar Kashani, B., Defrance, M., Vu Manh, T.-P., Azouz, A., Detavernier, A., Hoyois, A., Das, J., Bizet, M., et al. (2018). The transcription factors Runx3 and ThPOK cross-regulate acquisition of cytotoxic function by human Th1 lymphocytes. Elife 7.
Shapiro-Shelef, M., Lin, K.-I., McHeyzer-Williams, L. J., Liao, J., McHeyzer-Williams, M. G., and Calame, K. (2003). Blimp-1 is required for the formation of immunoglobulin secreting plasma cells and pre-plasma memory B cells. Immunity 19, 607-620.
Simpson, T. R., Li, F., Montalvo-Ortiz, W., Sepulveda, M. A., Bergerhoff, K., Arce, F., Roddie, C., Henry, J. Y., Yagita, H., Wolchok, J. D., et al. (2013). Fc-dependent depletion of tumor-infiltrating regulatory T cells co-defines the efficacy of anti-CTLA-4 therapy against melanoma. J. Exp. Med. 210, 1695-1710.
Smyth, G. K. (2004). Linear Models and Empirical Bayes Methods for Assessing Differential Expression in Microarray Experiments. Stat. Appl. Genet. Mol. Biol. 3, 1-25.
Takeuchi, A., and Saito, T. (2017). CD4 CTL, a Cytotoxic Subset of CD4+ T Cells, Their Differentiation and Function. Front. Immunol. 8, 194.
Tian, Y., Sette, A., and Weiskopf, D. (2016). Cytotoxic CD4 T Cells: Differentiation, Function, and Application to Dengue Virus Infection. Front. Immunol. 7, 531.
Waight, J. D., Chand, D., Dietrich, S., Gombos, R., Horn, T., Gonzalez, A. M., Manrique, M., Swiech, L., Morin, B., Brittsan, C., et al. (2018). Selective FcγR Co-engagement on APCs Modulates the Activity of Therapeutic Antibodies Targeting T Cell Antigens. Cancer Cell 33, 1033-1047.e5.
Williams, K. M., Hakim, F. T., and Gress, R. E. (2007). T cell immune reconstitution following lymphodepletion. Semin. Immunol. 19, 318-330.
Wu, T., Shin, H. M., Moseman, E. A., Ji, Y., Huang, B., Harly, C., Sen, J. M., Berg, L. J., Gattinoni, L., McGavern, D. B., et al. (2015). TCF1 Is Required for the T Follicular Helper Cell Response to Viral Infection. Cell Rep. 12, 2099-2110.
Xie, Y., Akpinarli, A., Maris, C., Hipkiss, E. L., Lane, M., Kwon, E.-K. M., Muranski, P., Restifo, N. P., and Antony, P. A. (2010). Naive tumor-specific CD4⁺ T cells differentiated in vivo eradicate established melanoma. J. Exp. Med. 207, 651-667.
Yang, Y., Xu, J., Niu, Y., Bromberg, J. S., and Ding, Y. (2008). T-bet and eomesodermin play critical roles in directing T cell differentiation to Th1 versus Th17. J. Immunol. 181, 8700-8710.
Ye, C., Brand, D., and Zheng, S. G. (2018). Targeting IL-2: an unexpected effect in treating immunological diseases. Signal Transduct. Target. Ther. 3, 2.
Zhu, J., and Paul, W. E. (2010). Peripheral CD4+ T-cell differentiation regulated by networks of cytokines and transcription factors. Immunol. Rev. 238, 247-262.

1. An engineered T cell for use in a method of treatment of a proliferative disorder in a mammalian subject, wherein the T cell has been engineered (i) to overexpress BLIMP1 and/or (ii) to knock-out or decrease expression of BCL6.

2. The engineered T cell for use of claim 1, wherein the T cell comprises a chimeric antigen receptor T cell (CAR-T), an engineered T cell receptor (TCR) T cell or a Neoantigen-reactive T Cell (NAR-T).

3. The engineered T cell for use of claim 1 or claim 2, wherein the T cell is autologous to said subject.

4. The engineered T cell for use according to any one of the preceding claims, wherein the proliferative disorder comprises a solid tumour.

5. The engineered T cell for use according to claim 4, wherein the tumour is selected from bladder cancer, gastric cancer, oesophageal cancer, breast cancer, colorectal cancer, cervical cancer, ovarian cancer, endometrial cancer, kidney cancer, lung cancer, brain cancer, melanoma, lymphoma, small bowel cancers, leukaemia, pancreatic cancer, hepatobiliary tumours, germ cell cancers, prostate cancer, head and neck cancers, thyroid cancer and sarcomas.

6. The engineered T cell for use according to claim 4, wherein the solid tumour comprises a melanoma or a sarcoma.

7. The engineered T cell for use according to any one of the preceding claims, wherein the engineered T cell has been engineered to knock-out or downregulate expression of BCL6.

8. The engineered T cell for use according to any one of the preceding claims, wherein the engineered T cell has been engineered to overexpress BLIMP-1.

9. The engineered T cell for use according to claim 7 or claim 8, wherein said BCL6 knock-out or downregulation and/or said BLIPM-1 overexpression has been engineered by CRISPR-mediated gene editing, transcription activator-like effector nucleases (TALENs) transient downregulation using short hairpin RNA (shRNA), small interfering RNA (siRNA), microRNA (miRNA) or RNA constructs for overexpression.

10. The engineered T cell for use according to any one of the preceding claims, wherein said method of treatment further comprises simultaneous, sequential or separate administration of an immune checkpoint inhibitor therapy and/or a BCL6 inhibitor compound.

11. The engineered T cell for use according to claim 10, wherein said immune checkpoint inhibitor therapy comprises CTLA-4 blockade, PD-1 inhibition, PD-L1 inhibition, Lag-3 inhibition, Tim-3 inhibition, TIGIT inhibition and/or BTLA inhibition.

12. The engineered T cell for use according to claim 11, wherein said immune checkpoint inhibitor therapy comprises: ipilimumab, tremelimumab, nivolumab, pembrolizumab, atezolizumab, avelumab or durvalumab.

13. The engineered T cell for use according to any one of the preceding claims, wherein the engineered T cell comprises Tisagenlecleucel or Axicabtagene ciloleucel, modified to overexpress BLIMP1 and/or to knock-out or decrease expression of BCL6.

14. The engineered T cell for use according to any one of the preceding claims, wherein the engineered T cell is a CD4⁺ T cell having cytotoxic activity and/or a CD8⁺ T cell having cytotoxic activity.

15. The engineered T cell for use according to any one of claims 10 to 14, wherein the BCL6 inhibitor compound is a BCL6 degrader.

16. A method of treatment of a proliferative disorder in a mammalian subject, comprising administering a therapeutically effective amount of an engineered T cell to the subject in need thereof, wherein the T cell has been engineered (i) to overexpress BLIMP1 and/or (ii) to knock-out or decrease expression of BCL6.

17. The method of claim 16, wherein the T cell comprises a chimeric antigen receptor T cell (CAR-T), an engineered T cell receptor (TCR) T cell or a Neoantigen-reactive T Cell (NAR-T).

18. The method of claim 16 or claim 17, wherein the T cell is autologous to said subject.

19. The method according to any one of claims 16 to 18, wherein the proliferative disorder comprises a solid tumour.

20. The method according to claim 19, wherein the tumour is selected from bladder cancer, gastric cancer, oesophageal cancer, breast cancer, colorectal cancer, cervical cancer, ovarian cancer, endometrial cancer, kidney cancer, lung cancer, brain cancer, melanoma, lymphoma, small bowel cancers, leukemia, pancreatic cancer, hepatobiliary tumours, germ cell cancers, prostate cancer, head and neck cancers, thyroid cancer and sarcomas.

21. The method according to claim 19, wherein the solid tumour comprises a melanoma or a sarcoma.

22. The method according to any one of claims 16 to 21, wherein the T cell is engineered to knock-out or downregulate expression of BCL6 prior to being administered to the subject.

23. The method according to any one of claims 16 to 22, wherein the T cell is engineered to overexpress BLIMP-1 prior to being administered to the subject.

24. The method according to claim 22 or claim 23, wherein said BCL6 knock-out or downregulation and/or said BLIPM-1 overexpression is engineered by CRISPR-mediated gene editing, transcription activator-like effector nucleases (TALENs) transient downregulation using short hairpin RNA (shRNA), small interfering RNA (siRNA), microRNA (miRNA) or RNA constructs for overexpression.

25. The method according to any one of claims 16 to 24, wherein said method of treatment further comprises simultaneous, sequential or separate administration of an immune checkpoint inhibitor therapy and/or a BCL6 inhibitor to the subject.

26. The method according to claim 25, wherein said immune checkpoint inhibitor therapy comprises CTLA-4 blockade, PD-1 inhibition, PD-L1 inhibition, Lag-3 inhibition, Tim-3 inhibition, TIGIT inhibition and/or BTLA inhibition.

27. The method according to claim 26, wherein said immune checkpoint inhibitor therapy comprises: ipilimumab, tremelimumab, nivolumab, pembrolizumab, atezolizumab, avelumab or durvalumab.

28. The method according to any one of claims 16 to 27, wherein the engineered T cell is a CD4⁺ T cell having cytotoxic activity and/or a CD8⁺ T cell having cytotoxic activity.

29. The method according to any one of claims 25 to 28, wherein the BCL6 inhibitor is a BCL6 degrader.

30. A BCL6 inhibitor for use in a method of enhancing immunotherapy in a subject having a proliferative disorder.

31. The BCL6 inhibitor for use according to claim 30, wherein said immunotherapy comprises immune checkpoint inhibition, an anti-tumour vaccine or a T cell therapy.

32. The BCL6 inhibitor for use according to claim 31, wherein the T cell therapy comprises simultaneous, sequential or separate administration of an engineered T cell as defined in any one of claims 1 to 15.

33. The BCL6 inhibitor for use according to any one of claims 30 to 32, wherein the proliferative disorder comprises a solid tumour.

34. The BCL6 inhibitor for use according to claim 33, wherein the tumour is selected from bladder cancer, gastric cancer, oesophageal cancer, breast cancer, colorectal cancer, cervical cancer, ovarian cancer, endometrial cancer, kidney cancer, lung cancer, brain cancer, melanoma, lymphoma, small bowel cancers, leukaemia, pancreatic cancer, hepatobiliary tumours, germ cell cancers, prostate cancer, head and neck cancers, thyroid cancer and sarcomas.

35. The BCL6 inhibitor for use according to claim 33, wherein the solid tumour comprises a melanoma or a sarcoma.

36. The BCL6 inhibitor for use according to any one of claims 30 to 35, wherein the amount of BCL6 inhibitor administered to the subject is sufficient to enhance cytotoxic activity of CD4⁺ T cells and/or CD8+ T cells in the subject.

37. The BCL6 inhibitor for use according to any one of claims 30 to 36, wherein the proliferative disorder comprises a tumour that does not overexpress BCL6.

38. The BCL6 inhibitor for use according to claim 37, wherein the cells of the tumour are negative for BCL6 expression and/or do not carry mutations in the BCL6 gene relative to the germline BCL6 gene of the subject.

39. A method for producing an engineered T cell, comprising genetically engineering a T cell to enhance expression of overexpress BLIMP1 and/or (ii) to knock-out or decrease expression of BCL6.

40. The method of claim 39, further comprising culturing the T cell under conditions suitable for expansion to provide an expanded cell population.

41. The method of claim 39 or claim 40, wherein the method is performed in vitro.

42. The method of any one of claims 39 to 41, wherein genetically engineering a T cell is performed by CRISPR/Cas9-mediated gene editing, transcription activator-like effector nucleases (TALENs) transient downregulation using short hairpin RNA (shRNA), small interfering RNA (siRNA), microRNA (miRNA) or RNA constructs for overexpression or by introducing a nucleic acid or vector into the cell.

43. The method of any of claims 39 to 42, wherein the engineered T cell has the features of any one of claims 1 to 15.
